SEQUENCE LISTING (22 sequences)

SEQ ID NO: 1 - DNA, 51620 bp, homo sapien
ccggagcaggaagtggtgggtcccagatgtgactcactctcattaggtagcgtggaaggagccttcggatgggtgagcctgggcgcgtctgaggaagggcaggcgggggccgggccacctccctgcagaccctggccggctgctggggccggggaggagagccaggtaaaagtggggtgtggggatggagtgtcaccagtcctagtggcttgagccccaggggacggggctggacagtgggtgggaagcagctcacccggcagcctggcctgggagtgcactgggcagggtctgggctgggggagtaggaggctccgtgagcctactgggcagatggctgggagaggcggcccttcaacaggggaccagaggcgacttgtcactggcttttcccagcctgacaagaaagtgcggttgtgaccacaatgttgtacatgtggcccgttccatctggaattctaggaaccacagactctcaaatgtccacactccttctcgagggtgtaattttacatttacaagcacacatgacttttctcaatgaaaggaaacattggcaagggaaggccaattcaactaattttatgaaattataaaacaaaaaacaaacaaaacctctgaaaggagtgcatgcctgagggtcaggaaaccaagatgcctccaaaaagcagactggtttccctttccctgcaatgcctctcctctatcccccagggaccgcatgggccatctggagtgggtctttctggagcgggtctttctggagctttctatgcattcatctaaacatgtatgtacagagggtgaggcagttgcctcttctctgtgtcatcacatcagggtcaagggcagggacatctgtggctgggggcttcctccctggacagccattgctggagaggagaagggagggccgggagggggaagccaagggcctggaggtaggggccaggccgagccacctacgacaggggccctgggtccaaggcctggacgggcagccagtgtgggctggatgtgtctggaagggctatgtgaccgtgggccttcactagcatcaccatcttgcagatggaggctgtgaggggtggactcaacaccagtgtttctcggagtgaggaccgggctgcccagggaatcacagggggttctagcctgtgtgtggaaggacatgtttaaaatctaagtatttgtcttggggaaaaaaaaatctgtaaataggacattgccattgtgacatcatgactgtattattgtcttagacgaggccaaaggagacataaaagtgacaagccagccaatttgagaggaagtgccaagtgccagccctgcaggaagtgcaggaggctgagggggctgcagctcaggaaggcgtttgctgcacagccttcgcttcccacccagttgtctgtcttcccacctccagaggccctgggttcccaaggtttacctgcagctcctggtgcagagtgtgggggaggggaggctctcagattgttccagattctgtggccctcagaagggcaggtgtgagctttgtggaggtagcgtctccggtggtattttctttctttcttatttttatttattattattttttgagatggagtctctgtctgttgcccaggctggagtgcagtagtgaccctcttgggtcactgtaacctttgcctcctgggcttaagccgtcctcctccctcagcctcctgagtagctgggactacagttaccacaccactacacctggctaatttttgtatttttagtaaagtaaagacggggtttcaccatggtggccaggctggtctcaaactcctgacctcaaatgatccatccacctcgacctcccaaagtgctgggattataggtgtgagccaccacccccagcctcttttctttttttaagacaggatctcactctgtcacccatgctggagtgcagtggagtggtcatagctcactgcaaccttgaactcatgagctcaggcaatcttaccgcctcagccttctaaagtgctgggattacaagcatgagcccgacgcccagccttctagtgctgttttcatgaaagaagcagctgtttactagcagctgttttccgggtgatgagggtgaaggtggtattgcagaacatctttgccaggagtaggtctgggaggtttgcttgagggcatggaggttggtgtagggagagctcaggaagtagaaattgggctggagtgtggcctctggacaggccacaggagagtctcaaaaggagaaaagatcccagcactttgggaggccaaggtgggcggatcacttgagcccaggggttcgagaccagcctgggcaacatggcgaaaccccgtctctacaaaaaatacaaaaattagctgggtgtggtggcgtgtgcctgtagtcccagctacttgggaggctaaggcaggaggatcctttgagcccaggagttggaggctgtagtgagccatgactgtgccactgcactccatccagcctgggtgacggaatgagaccctgtctcaaaataaataaatttttcaaaagagacaaaagtccgactcgatggttgcactggggacatgcaccccactctacaatttatagctactgatttgacccattccccacctcctccaccgctccatatggtaggtgccgttcacctccagtttacagaggagacagggcccagaggggctgtctatcctgaataacccagcagatcggtcctcaggcatccagttgtgcccaccacagtgtggtctgctgccacatttgagcctggagtcagggccagcacactgtgtctttggcgagatcagatggggcaaagctgagaacaggggctctggttggtgctgagggattcgtgggggtggtccttactgggaagatgaggaattgggctcctgtgagggcctcccctaccctgccctgtgcctggacttcgctgggcccactgcgggagagaagaggtaggacgaggccttctgcgtggctgggccagggtgggctgtctcacgtgctttcctggatgtgtggttgggtcatgccttagccaggaggtaggtggtttgtccactccctgtcattgcaagggtcacagaggtggctgggtggcctgaccctcagcaccgctccttgcctccctcctgctgtcctggaggaagaggatgtggacttgttcatactgaggtgacacagcatcctctctcctacctctcccactgcccagggagagagcagcgcttaactccgctggagaaagttccaggcctttgagggagcatgcagagtgggtcacagtggatgcctgtatcagagcagcccattctagcctttcatggggtgggagggaggcatttactacttttaaaagacacgtgtggccaggcaccgtggttcacacctgcagtcccagcgctttgggaggctgaagtgggcggatcacttgagctcaagagtttgagaccagcctaggcaacatggcacaaccccaactctaggaaaaaattacaaatattagccagcatggtggtgcatgcctgtagtcccagctacgcaggaggattgcttgagcccaggaggtggaggctgcagtgagctgagatcatgccactgtactccactctgggtaatagagcaagaccctgtctcagatagatagatagatagatagatagatagatagatagatagatagatagataaacagatagatagacacatgtagccaagagctggaagttagttggaactggagaatgagaccacatctgaacaccagccagagaagcaggggtcttccagaggttatccggtccagcatcccgcctccaggcagaactggactgtgctctggtccggcggcacgggttccagctctgttctgcctcctcccaacagcgtgtgagatgagcaagggatggagcctgtttcttgcacctgtgaaatggggttggtaaaggtaccccctgcatcaggtggttgtaaagattaaatgagctgatgcacactctgctcctggcacagcggaggcgctacctaagtggaagtgtgcgtcactgtcggtgtcgtgaggctctcgtctgccttgtcctgccttgggtgggagattggacctccctgggggagcctgggtggtgcaggcaggcagggctgagctgagcagcctccctgtcacctacatccctcggactaatcattttctttctgttcctcagtttccttatctgtaaaatggggctgatggcagtgctcaggtaggttaatgggagggctagctgggtgaaaacccttcccaggcacctgacactggtgtgcccaggctgagaggttgccacacagtggctggagcccctccagctgtgtgtgccggggatcccgccagccccctcatctgtgcagtggccccctccccaccaaggacagtagcatggtgtcacccggcttgccataggcatctggcctgacctgctgctgcacacggatttctcaactggccacatcatggctagggcattgcccaaccctgagtgcagacctgcttggccgaagcaccctccgttccctgctctcccctggaagcctggaagcctttctagaagggaccagtgttggggtggagaagggttgggtggtgagtctgggaccagagctccgttcctctttggtggcctttttccttggaatacctaggaccgtgctttcctgccgccaccagctctctcattttttattttgacagggtctggctctgttgcccaggctggggtacagtggcctggtcatagctcacagcagcctcgacctcctgggctcaagagagcctcccaccttcacctcccaaagtgctggggttacaggcatgagccaccacacccagcctgtcctctttgatgggccaatttcacaggcattttctgagcacagcacagcccagtgctgccgagatgaacctgagcagactctgtgttaggatggggtggggacggctgaccttgggcctgggagaaggtcctccctgttctagagcacagccatctggtcagtttctcctggcttgtgaggccctcccagaccgggcctgacttccctccccactcccgccacttgtcaccgttatccactctgggccagcccccctgacattttcactcccagcttggctatctcctgcccctttcctgcccttggcccccacagttcccactgcctggggtgttctccccaactgctgcttggctgtgtcctccaggtgagggcctccttcaggggtcctccagcatggggagaagaggggacccagccggctctgctcttgagctgttcactcagccgcctcccccacccccttggccttgctttgagcccctgtcttgctgtggctggggcccaagggacgccgggataccctcttctcaccatcctccctttatgtccctgtttcccccaccaccgctgaccactggaagggtgaacccacgactgggcctgagggaaagggaagccaagggaggtctttggtgcaggtacccccgtttccagttgtgtttcctggccccaggctctgcccattcttgcctcctcccagggtatcaggacatgctggaggtgacagggcagaaggaatggccatgaccctcagacacaggcctgtaccctggggaggagctcatgtccctggaaggcagaaggtacctgcagtgggtgatgccaccacccgccttctagggcccggatccctggaggccgctggacccagggcaccctgatcggggtgctttgtcttccaggccccaggctcttcctgccctccagggtgcccttcctgcccctgtaactggccagctttcctcatgctggcctctgaaacaacctagtcacactgcctcgggcgcagctgactgagatcattgcttcctggagtcaccgactggctgggcagggagaagcctagcaccctgggatgcagctgctctgcacagatggggaaactgaggccctgggagctctgcagccttggggctggtgtgtcttcctggtgggagctatgttggagttgcaggccctgccccattcacaagcacatcccttggcctcactcatgggagggatgtggactgggggccagtcttgtgggcttgtcttggtcctgctgctgttcttgggagtctgggtctccctcctcctgggcatctccttgctggagcaggggtccagggtgctggtgtaggctgggatgagacctccttctgtgtgtgtgcgcatctggctcctgggcccccaggcctgtctccggtaccccctctggctgggaggaggagcccttttaagctgtctgaaggagctattttaaggggggcctgggctaatgggaagtggcctcttttgatatacccagactccctggagacactgctccatcccctgggggcagggaccagcacttctgtatccctttgtcaggctcagctctcctgggcatgggtacaagagcagagctgccctttgggggcaggcaggaagtgaggctgagaccctggtactgaagtgtgtccccagtgcctgtttatgtcacgatttctcaggctgctcaccctggtgtctggagctgactggggtggccctgggtgatggtacttgctgtgtgcccacatctacgtgaagcccctgagaactggtattgttaccagtccattctgtgggttaggaaactgaggcccagagaggttcaggaacatgcccaaggtcacacagctcctccaaggtggagctgagattcacggcctgctgttggatctcagagcttgtgctcccaaccaccatgctttcctgccttccccttctgccctgccctggcatttcttttcctcacttggctcatttatgacaatctgccctcctcctggcatgagctcctcaggggacccatgcctgtcctgtgtgttgtagcagcagcagtgcctgcacctgctgcaggcaggagctcagtggcacttggcagatgggggtctggtggcaccaacatgtggcctctttcctggtggcaggatgggcccctttccatgtgcaatgggggtttatgaggttttatagctatgaggtcacgtcactgctagaataggccatggagcgaagagaagggtttcatccccattgctcaggtgagaaactgagtcctgagaggcaggtcacctgccagagctcatgcagttgggactggggattccagggttggggcccagtccttagctgccagttcagtgctggctgtaccccccagtctgtggagtatggggtttgatgtccctgtgtaccaggcagggttagatcctgggagctgggtagtgcatggctggacgtgcagccaggtagtgacaggtcagggtagcctgtgcttctgtgggaggtgcaggccctggggaagctaaggaacaaggccagactgggaggggaccaggcagccttcctggaagaggtggcagctcaggttggccctgaagggcgggcaggagttcgccaggcagaaggcatttggaggtgaggaggtgggcgtgtttttagattgccggagctggagatgaaagggtggggcttgtttggggaaccgaaatcagccaaatcacgcagccacagaggaggccagggaagctctagaggcgttttttggccaaggggtgagcagagatcgggcttggtgttttggggggtcctgggtaggtgtgggaggcctcgtttcgggggccacggggctcctgtggcgtgagactccatcacagtgacctcctgtgcccaatagctccgtaagggcagggtttgtggaaggaaggagtgagtgaccgggaatggggtcttgggctccagggacagccatggcgagggctcttctgtgtgggtgcaggtccggaagctggggctgtggttggtgggggaggctgggtggggctgcttcccctgcagaagggctcttccgggctcttggcgcggtttcacgaggcagtcccctctgtgtctctcgtgccccctaggcctggatcgggttctcaggaagcctcggggaagtgagggtcttagcgccttggtttggggctgggcactttgcgagctggtgtcatggaggccaaactgggggcctccgggccaggtggtggcttcaggcttgctgttgggacaggaggccaggctaggcagtgacgcctcactgagctccatgggtggcacgctgagctctgggtgggagaggcctctcccactgacctctctgtgactggcttctctgctggtctgagtgggaagggccctcagagaccctggaggcctttcctgccctggaggagtcaggcccagagagggtgaagaattgcccgagtcacacagctggtgacagggagagctgggactgtaccttacatcacctgacattcagcccagggctctattccctgcactgtggcagatggggaaactgaggcaggtgggccactcctctgcattccatgggagaggcaggaaattcgacaggagccctttgccgggttggcacttggagctgttttcctgcttgcttttccccagggcaaggtgcttcccagctgaaaactgcctagggagggtgctgttgagaccgtgagcacctgagagccctggtctttcactcaggcctctcctctcacctccacctgggaggctaagctcagggtaggtgtcctggcccacaggcaggtgcatgggctggtcccgccagcacctgcaaccttcttgtgcgcagctagcccaacctggggtcaggtggaggtgggtaagtggggcccccagcattaaagagcttcataggctgtcaattaaggggaggctagtgggctctacaggccacagctacccagggagccttcttaggggaggagttcctaggtgggtctcagagactccaaagtcagcatttcccaaaggaggttcattagggattcagtgcaaaaagagtctcattaacaaaaggatggggaaatgttcagctgtgtttaattcagcaggcttctttcctgcaggacttgtcagagcctttaatttgctaatgtgtatgggggatctttaaatggggaagaggagaatgttgagctcagcaccacctgtgaccagaaatggctgtggatgtgtgtggctgcgcatatgtagcttttcatgttcctgcttttgggcagaatgcacctcaggaaatgcagatagagagttaatctgcactgggccttggagaggggcaggcctgggttcgaatcctagctccactgtttcctagcaggtgggctttgggcttggaggttaaggtcccagagtcagttgccctggctagaaatggagcgagagcacccccgcccaccacccttgggtagcccagcccattcatggttctctgcaggttctcagaggagacagccagccaggtggtttttggcccagtttgtctcccggctgggctcagaggtcgcagagcttgtcttgaggcctgggtactgtgccgtctcctgccagcaagcctggaaggcttgggccgaggacccgactctgcctgggatgtcacctgtgagctccctccagctcagaccaatatcaccacctccctctgacctgggttgggtgggacggctgctcccttgaggcccaactaggccctacatggaggggtcagcccacactgccactcatggccctggaaagccggcctgagttgctgaccaatgttctctctacctcccagaggctgggcactcacagaccagagtaggggccgggcctcctcacccccagtgctgctggctcatggcagggttgaggggtggggacaggtgccgggccctctcacttctgcaccccatggccctttctgacatcttggtgatgcatgccatctagactggggcattttgcgccaggggtggctcagtacactgcttgcatcacccccaactgaatcctttcaaaatcctgtggggtaggactgttattctccttttgttaaaatgaggaaactgaggcacagagagtctgttgggtcatgtaatgtattatttgttttaatatttaatttatgaatgagtgagatggagtcttgctctgtttctcaggctggaatgtggtggcacaatcatcacttgctgcagccttaacctgaactcaggaggtcctcctgcctcagcctcccaagtttgctggctctacaggcatgtaccactgtgcctggcttagttttaaaatttttttagagatagcatctcactacgttgcccaggctgatcttgaacttttggtcttaagcagtcctcctgcctcggcctcctgaatagctgacatcacaggcatgagccaatgtacctggctaggtcatgcaatttaggtgtgatgggcctgggattcatttttcctattacacatttttttttttttagtcacataagcaacacactgacaataggaatagaaactgaaaatgagaagtgggatttatccactctgtgaaatcaagagttttggtttttctgtgtcctggttccatccttatttgttccaggcattgccatgtggatttttcacaggataccctgtgacgttagtttcaagtggcctcaggctttttgacagctgcatagtattccattgtaaggttgtatcataatttatttcaccagttcgtgactgataggtatattattttcaatcttttgttattacaaataatgcttcaatgactaatcttgtacttatgtcatttcatacttgtgacaatgtttgtacgatacattactcaaagtggatttggtaggtcaaaaagcaaatatgtccttcagtgactttgttaggtattattaaactaccttcactgggggttgtactgactcacattcctgcccaggagggaatgtgagtgactgtttccaacacagacttatgagacttatgaaatgctttgatctttgcccatcagataggtaaaaatggttttccatggtagttttgtttttgttttaaagatggggattttgctatgttacccaggctagactcaaacttctgggctcaagtgatccgcctgcctcagcctcccaagtagctgggactataggtgcatcccattgtgcctggctaccatggtaggttttttttttttttttttttgaggcggagtcttgctctgtcacccaggctggagtgcagtggcacaatttcagctcactgcaagctctgcctcccagtttcacgccattatcctgcctcagcctcctgagtagctgggactacaggcacccgccaccacgcccagctaattttttgtatttttagtagagatggggtttcaccgtgttagccaggatggtctcgatctcctgacctcatgatccgcccacctcggcctcccaaagtgctgggattacaggcttgagccactgcgcccggcctaccatggtagttttaatttgtgtttcattacgagtgtggttagcttccttccaaatgctgtgggttggttgtgggttgcttttttttttgagacagagcttcactctgtcaccgaggctggtttacaggcatgatcttggctcactgcaatctctgcctcctgagttcaagcgattctcgtgccccagcctcccaagtagctgggattataagcatgcacctccacacctggctagtttttgtattttttaatagaaatggggtttcaccatgttgctcaggctggtctcgaactcctggcctcatgtgatctgcctgactcagcctacctaagtgccaagattacaggcgtgagccaccatgcctggcccttccaaatgtttaacagtcatttagattgccttttctatgaactttctcttcctagtcttctcccatttttctattgggctattggtcttcttttttatttgtgggagctctttacatatgaaggagattagctccttgtgatatgaatcacaaatgttcccctaatctgtcacgtgtcttttgattgtgcttttggtaatttttaccatgtatattttgttttttcatgtaactgaacttatcagtctcttctttatggcttttgggttttgtgtcaggataaaaaggccatattcactacagcatcatatgagaattctcccatagtttcttgctctctttggaaataatcctggtataagttgtgaatccagcttgcttgctgtcttgtggatgctgcctttctaaacagaatactagcagcctaagagttaagactcaaatgcacatctttcctgtccccttcaggacagtggttactctgggttgcatctgtccatgggcccgtgaaatggatctctctctctctctctttttttttttttttttggcagagcctcactctgttgcccaggctggagtacagtggtgcaatcccatctcactgcaacctctgcctcccgagttcaagtgattctcatgcctcagcctcgcgagtagctgggactataggcacgcaccaccaccttgggctcatttttctatttttagtggagacagggtttcaccatgttggccaggctggtctcgaactcctggcctcaagctatccacctgccttggcctcccaaagtgctgggattacaggtgtgagccaccgtgccaggtctatggagctcttcttgtacctcccagccccattcatgtcttgctgtcactgctggacccactgtcagggtggggccctaggcaatgtgggaagtcagccagcaccctcttgtcctgggcaggacgagaaccacgtgaggtcccccatggccagctggacttcccctttctgttcttccttatcaagggaaaagggatcagaaatctgttaaatgtgggcgtgaaagtgtttcccttctgaagggggaagctagtgagggcagtgcttaaagaaatgaatcactaattgaatttcaagcaagagtgaggaaaaagtagggctatttctccaacaggggagcctggttgattgcttagtcagttgataaacactgttgcctgaacacccgctgtgtgcagagacctagcagataccctggttggggcagtgctaaggattctgccttctgggagctcatttcctgagcagaattccctcttggtacctggcctggcacctggtgcacagaaggcatgagtgcacatctgctgaaggtatgcatggacgaactctccctgggcagtcactaactagcattagaatgggcatgagacatagtggacctggattcattatcattggcagctggagccttgggggtgcattttaaaggggagattgatcagggtaatgcttaaccaaatgaatgaagtggaggggaggatttcaggaacaaagaacactttttttttcctttttttgttgtgtctctggcagtttttggtaacaagatgatgctggcctcatagaatgagttagggaggagtccctcttcctcaatttttttgaatagtttctgtgggaatggtactagctcttcttcgtatatctggtagaattcagctgtgaatccatcaggtcctggccttttattggtaggctatttattactaattcaatttaggagctcattattgatctgttcagggaatcagtttcttcctggctcagtcatgggaggattgtgtgtccaggaatttatccatctcttaggttttctagtttttgtgtgtagaggtgtttgtagtagtttctgatggttgttttttatttctgtggggtcattagtaacattcctgtagtcatttctaattgtgtttatctggatcttttctcttttcttctttattagtttagctagtggcctattttattaattttttcaaaaaaaaaaaaaacaactcctggattcgttgatcttttgaatggttttttgtgtcttgattttcttcagttcagctctgatttttgtcatttcttgtcttctgctaggcttggggttgatttgtttttgcttctctagttctttcagttgtgaagttaggttgttaatttgagatctttctaagtttttgattcggacatttagtgctatgaatttccctcttaacactaccctagctgtgtcccaaagaatcaaagaacaactttctacccatccctagaagtagctctctggggccaggtgagaacatcagccttttccctcagtttttctccagttgcctatccttttcattaactgattcagcctccacccccagcccagtcccaactaggccagagctggcctcagaggtctgacacagcccctcctttgcaagaggggaaactaaggcacaggtcaggcaagagacttgtttagggtcacagagcaggttggtggctaagctgggactggaatgcaggtttctggtccccaagactgatcagcagcctgtgccctgggagtgggaacaaagtcagcctgggggtgcttgggggctatggagggggagcagggttggctgggggctcatgccgttggggaatttcagtctttgcccctgctcggcaactttgcaggctgttaggagatgcggtggggggcctgcccactctgcacctgcctctccctggaccccatggttgggctctgaccctgcagggcttcctctgacactaagcgtgccttcttggccatttgccaatttgtcttgcaagcttggcctgttggacatcatgacctgtgcagacaacctggttatcagcacccccagtttactgatgaggaaactgaggccagagtgtgcaggcgactgctattcacaggccattggtagcagagctgggattgggtgcaggcatcctcccagaggccaagcgtagctgggagaggtgtgctgtggcaggtaaggcaggtgcggcaggtgcagcaggtgcttcccgatcttgagggaggccctttcttcatctgccctcatcctttggctgtggcgccctcctccctcacccatgccactctgcgtgcttccccaccctgggcctggtgcaggattctggctggctaccctgctcggccattcacgtcacaggtgtttgccaggaccagctgcgggacacacacacacagatcccaccacgacagtttggcaagtcaggggccgggatattggacccacactgggaaggcctcttggtacgggaggtgcctgggagagcagggcatgggggccacagggctctggggacagagagggctgggcttggggggatgggaccttgaataccagcaacattgaatccctgggtcccacgaggtttactgggggcctcttaggtcaggcttgcactgggcagagcagctgctggcttcatggagctcagggtctcttgagagagttcgcgttaatatgagattcccttggcaaatgaaaaatctgcctgagacccagcctaccaggggagccgagagccggcaggaccgggggcgcgagacggagcatgcaggttttttgtctggagctgcgtggcgtcccaggcagggtgtgggcccaggggcctgagcggggcctcttccctggaggcttccatgcccccctcactccaaggtccccacttcccctttttgaggccaacagggccccaaagcagccctgagcctgctgaggctcggccggaggaagccaggctgtggtttctgccaacagtggccgggtgaggaagtgggtgtcctgtctgcatctgtgaaagctgggggtttctagaacatactccctgcaagggctgtggttgtgctggggacgctgccagatcagctggactggggaaggattttccgagcacccagggacctggccctgcagttgcagtatggagtcccagcctcttgcttccagcaggtcttctggctggagaacctggacccagccagccagtatttgataaggagaccacagtcctgccttagttgcagggaagggataagggagtgtgtgtggggttgctcagcaggcttcctggaggaggaggccctcctggagttcgctggcctgaacttgctcctgcaaggcagagggaagtaattcgatggggtcccctgagctccccacctcttggtcatagctctgtcctgggccttgtttgtagggaagaggttggtgttttgggatattatgggatttctggatgccagccccatgagggtgatagggaccccggatatcctgtgtttaaatcccagctttttttttttttccagacaggattctctatcacccaggcaggagtgcagtggcatgatcatagttcagtgaatgtagccttgacctcctgggttcaagcaattctcctgcctcagcctctcaagtagcttggactacaagtgtatgccaccaagcctggctaattaaaaaaaaaataaacaactttttatagcgatggggtctcattatattgcccaggctggtcttaaactcctgaactcctggctggggctgggctgtggagcctggaaagtgtgatggagggatgagcggggctgtcccctccaagacccagctcctttctggaggactggctgtgtctgcaggggcacactcttacctctgtgctgactgagacccttccagttgactctgaacccactgaggggccacgggtgcttgttagacgcaacaaactgggtccagaagcctgatgtggactttgagactcttggttgcgagtgacagaagctcaaactggctgaagctgaaaggggcattgcttgtcccatgtggttaaatatctagaggttgcgtaagcttcaggcacagctggctcctgtgaccccaggaggtgttgccaggcccctgtcttgtgccagttctccatgttggtgtcagtttcaggtgggctctctccttgtggtggctcgggtgtctgctgacagtgtcaggtcaccgacaaaagagaacgcctctttccctgggtcagcagaagtccaaagagagccttcacttcccaggcctgtggcattagggcttgagagggactttgctgattggccacactggggtcatgtggcccatgaaccagggtactgagggtcgggagaagggacattgcacagcagaagcatgaaataccctcctcccatgtggacaccttccccacaggggagtcccctcttctttaaaatggggctgctcaccctgctctaccactggggtggtcgtgaggtgagagtaagtaagggaccccctgggactgcagtctcctcaggaagaggggacgtgatacctctgctcttggtggctttggagttcagacaggacccatgcggtgtgggtgagggagtgacagggaggcagggcttgccttgagctaagggaggactcagggccagaaaaagtgcccagtgatggaggagactgccctgagaggtggtgagcccccccgtcatggaggtgtgcaagctaacagcagggtcattcaatccgggagccctggattggatgtgtctcagacatggcagaaggattcctttatccagggccttgtggggaggccctccctgttgccaactgagtcgagggctggtggaagaactggtgattggacaggaggccttgaaaacttcccgggcagggggtgcctctgggtctggctttggcggggtggctcctagggaggagctggcctggggacatgccactggtggtgggttcctgggccccctctggagatgtgatcagtgagttcattccccccaggcctcacggtgcccggcttgttccaggagctggccaaggtccctggatgggctgaagtccttgtgcccatgttgtccactgtccacctgcgcctgcctgccgttgcccagaaagtggcctaatgagttccttgagaggcgtgtttaccaggcctgtgttcccacgggcccttgggactctctcagctggttcctccctttcctcgtggtggcccagcctggctttgtgtgcctggagcgggtgtgtccacacactctcacacttgcactttcatgcacgtgtctggccgcctcagctctggtctgggggctttgcctgggagatggagcagttgtggggagtggcctcagccctctggtgtttggagcttctcagcctggctgtcctggtgcactggtgtttggcagcaggccaggggtcaggtgggtttgctgtttgtgtgctgtggcatctggctcagtcccccgggatgtcagtgggcaagcctgggctgaggagccctggtttgggagctggcagacctgggtttgcatcccagcctatctgtccaccagctccagcagtgtgatcttaggcaaccactctgcctctctgggctcagttctgcatctgtgagacggtggtgccaccttggcctttgccggttgtgaagatgagagtgaatgtgtgtgcaggtgccgggggtgacaggaactgctgctgttattactgcaaagggctattttggggtggggggtgctgaattgaatgtatctcaggcagggaccttatcttgtttggcaacccccacagcacagggttggatgtatatggttagcacacagtaaatgcctcttgtggaatacagactgattagtttagatttagaggttgtaaactagtgggctgcaaatattgaaaaatttgaaatcagctgccaacatttaaaaatcatcatttcacgtagaaatcagatttgtagcctgtctgagcactttgggcactttgacagctcttggccacatccctgaagggttgcccctcgttgaacaggtgccaaggcctgtagacacctgcccaggcactgaaggggatggggcccccagcaaatgaccccttcctccttccccaaatatgtggttttaccattttgttcctggtgtgtggtgatggaataggaacttccagaggaaatagagcctgaggtggctcctggaattgaggtggccccagggatctcatactagcaggtgctggttggggacttggggaccctggatatcctgctttttgcctttgggggctgggggctcacctggccatcttccctccgctggaccagctgtcctttgaagcttctgctggcggctggccatttgtgccctgtgactgctctgagctgtcccgtgtctcacagcctccctgccctctgcctctggccctgtcctggttccaatctgaggcaggcctggaggacactgggggcgtggactcttggtccagggttgcccccttcatgcaaggttcctgctgggaatatggcagctgtccccaagtccccattgctgtgtgagggattgcacaattttggtaattttcctacctttggagactgatggggatccctggggagagagagcccagactagcacacaggagatataagggctggcaggcgtaggtgtgattcacaagcctttttggaatcaatgaatatgctaaggaatgaatggtgctggggaaagaaggaacccatatgggctgagcatggcaagtgtggggtcagctcacagtgttgtcacaaaggggccactgtgtccctctctgagactcagctcccttttctgtgaagcacggcttgcagacatctgaaatttgtatcagtgggctatttaaaaaaataaacaagatttttgctgggtacagtgcctgaaagcttaacacttcggaggctgaggagggtagattgcttgagcttaggagttcaagactagcctgggcaacatggcaaaaccccatctctacaaaaaatacatggcctactgcagcctaagtctccccaggctcaggtaattctcccaccttagcctcctgcatagctgaggtcccagctacgcaggagtctgaggtgggaggattgcctgagccctgggaggttgaggctgcagcgggctatgatcacaccacttcactccagcctgggtgacagagtgagaccccatctcaaaaaaaaaaaaaaaaagagagaaagaaaatgcagttttatgtggagcttactgtaacacagatgcagggggctaccctggctgaagcaggcatgggggcctgagcctatgcccccccaccttcccttcccattgggggcctgatgcatgtccccaaatctcagggctcctaggatcacagcttggaaccctctgtgcctggaggcatgagactcactggaaaatgaagtgaatgtggtgattgcaccaggaccatggggacagagcagagcctggaaacagacaccatctgtgtgtgatcacctgacttatgacagaggtggctccactgaaattcagagagaggatggccggttcactgaacactgctgggataattcaatatccatatggaaaaaagaagtgaaccttgatccctacctcacaccatacccagagtgaatcgaaatggatcagagacctttgtgtgaaagcaaaacagtgaaacatgcagagcagagctggcctgtagaagcttctgtgatgatagacattctctgtttatctgtgctgtccagtacagcagctgcatgtagccattgggtatttgatttttttttttttttgagatggagtctcactctgttgcccaggctggatgcaatggcacgatctcagcttactgcaaaatttgcctcccaggttcaagccattctcctgcctcagcttcccgagtagctgggattacaggcacacgtcaccatgtccggctaatttttgtatttttagtggaggggggttttaccacgtgggccaggctggtctcgaactcctgacctcaggtgatctgcctgcctcgacctctcaaagtgctgggattacaggcatgagccaccatgcctggctggctgttgagtatttgaaatgtggctagtgtgactgagggactgaatttctaattttattttaaattaatttaaatttaatttaaaatagtggctgggcatggtggctcatgcctgttatcccagcactttgggaggctgaggcaggaggattgcttgagcccacgagttcaaaaccaccctgggcagcagggggagacgcccatctctacaaaagagaatttaaaaattagctgcacacctgtagtcccagctacgtaggaggctgaggtgggagaatcacttgagcccgggagtttgaggctgcagtgagctatgatcgtgccactgcactccagcctgggcaacagagtgaggccctgtcttgaaaaaaaaaaaaaaaagcagccacatgtggctagtggctactatcttggacagtgcagtcctagaagacacaggggactagctttacgacaacaggacataaaagttaggttagggttaagatggataaatcagacttggtgctgtgaggcctgggccccgctctccctgatgctagccctcccctgacttggaccattaggtcaagttcctgtctgcttcgtctccgcagagctgaggaactgcgtgtggagtcagcccagtctggatgcacaggaggatgctggcggcacagtgagtgaggcctggtgccagagctgtgcggaccccttgttggccatggagcagcaggcccagaggccctctccccagccctgcttgcctgcctcggagaggacagaggcctaggcccacgggggagggtgttggcagacagatgccctccaggccctggggcctccttaacggccccttaacgacacgcgtgccaagggtggaggatgccagccaaggggcgctacttcctcaacgagggcgaggagggccctgaccaagatgcgctctacgagaagtaccagctcaccagccagcatgggccgctgctgctcacgctcctgctggtggccgccactgcctgcgtggccctcatcatcattgccttcagccagggggtgagtgagggcagcccctgggcttcacgtctcggccccaaccttggccaagctgctatcttctcttagcctcttctgtaaaatgcttatcttctgtaaaatgctatgcttttttgtttgttttgttttgtttttgggtttttttgagacagagtctcactctgtcacccaggctggagtacagtggcatgatctcggctcactgcaacctccatctcctgggctcaagcaattctcctgcctcagccttccgagtagctaggatgacaggtgcatgccaccatgcccagctaatttttgtatttttagtagagatgggctttcaccatgttggccaggctggtctcgaactcccaacctcaggtgatccacctgcttcggccccccaaagtgctgggattatagacgtgagccaccatgcccggccaatgctctgtcttttacagcacatttagactggtagaagacaagtttctaatttaaaaaaattttttttgagactgggccttgttttgttgcccaggttggccttgaactcctgggttcaagtaatcatcctggcttatcctcctgagtagctgggagtacaagcatgcacgaccatgcccagcctctctaactttaattcagttttgaagtctggagtggtttcagacattacattaacttgtccagataaactactctcatttttctccatgatgaaattcgattctactgaagacctgggtcagggatggcctgtgtatcactgccctcacttcctgtacccatgcagacatcattaatcaatccctacattccacactgagcctgtctacattctcagaatcctccttttgttttttttttgtttgtttgtttttgtagagacatggcctcgctatgttgcccaggctggtcttgaactcctggcctcaagtaatcctcctgccttggcctcccaaagtgttgggattacaggtgtgaaccactgtgcccggcctcagaatcctttttaacacagctctccaggctggcaccacctatgtcatttccggcccaggcacttgccttggtacagccactagctgtgtttagctattgagcatttaaaaagtgggtaatgtgactgaggaactgcatttttcaaaaaaaagagttttgctcttattgcccaggctggagtgcaatggcaccaacttggctcgctgcagcctccacctcctaggttcaagtgattctcctgcctcagcctcccgagtagctgggattacaggcccatgccaccacgcctggctaatttttgtatttttagtaaagacagggtttcaccatgttggccaggctggtctcaaattcctgacctcagctgatccacctgcctcggcctcccaaagtgctgggattataggcgtgagccaccatgcccagcccattttctttttagtgaatgtaaatttgattagctgagttctagaagaacactcagaaactccagggtgtttatgtgagtgaagtggccaggacctagcagctgccagtgcagcctggatccatgtggctaccaaccacaggatgcttgctactgtgtgccagcccccagatcaggcctctgcccccacggacctcccggatgctgtgcccgtcctgtcccacctctgtgccattgcctcagctcctcccctgtgggatgacttttcctggtatccctcaccagcctctgctcactcactgagggccaggcgcagtgtcctctttgggcggctttacaaggctctgcctgtgaatgagtcctgccctgtgcttgtggtatcacctgcggctgtgcacagagcaccgtgggaacccagagaggaggcagaggcagatggagagtgatggcatcatgggcggggctgtgggtgatgctctggttcccgagatgatgggaagggggtggttgaggcagccttggcagaggtgacaaaggccagaaggtgagtgggaaccaggtgtgggagggtgcatccacacccttcacgtggaggaagctgtaagtgaggcagccggcccggcaggctttctggaggaggtggctctgcactggggaaagccgaggcattcctgtctgtttgctccacccaggacccctccagacaccaggccattctgggcatggcgttcctggtgctggcggtgtttgcggccctctctgtgctgatgtacgtcgagtgtctcctgcggcgctggctcagggccttggcgctgctcacctgggcctgcttggtggcgctgggctatgtgctggtgttcgacgcatggacaaaggcggcctgtgcgtgggagcaggtaacaggaactctggactccctgccagctgcgccttcagcagctcccatccccgtggttggtgggcatgccacaggcccttcgtgccccacgcttggctgtgtgtctaggatggccccaggctggttttgtccctgtgagttctctgcattgacagggctgaggtggggagagcatccactcggacgctgggctgcagcacttttgggtggctccagtttggttggagatgacagaacttcagctcaaagaggcaaaaaggcacaaactggttcctgtgtcaggaaaggccaaggggtcagtgctgggctccagtgggtaactggaactggggcctctggacagcttcggccccgggcacgctgtccccaggtgggggcagagaagttcacagcggctccaggctgcccaccccagttgagagagagggccctttctcaaccagctcgacctgcaggcttgagccacactggcctggctgtggtcatgggcccagtcccagagaggctgtcaccaagcggtagcacactctgaccagtgggcctgggtcctatgacagctcctgtggctggggcagggtggggtgaggcccaaggaaaccatgtggactgaaggggagggagagggcagcactcccagaggggtggacgcaggaccagccggtggggatggtggggcagctctgggggtggagggagggcgggtggggccgcattgctggaggcagctggtgagagggtgcctcagcctcccggtaatcatttctgctgagcgagaaccacgtggcaggggcaccaggtgggggctccctggatcacgcggggcatggagcctcccacctagggtggccacaggcagtggatcagcacaggaaaccatgaaccttgtggccgggcccaccctagaaacacattatggcttttcttgttcccttgtatgtctgcccacgcagggggtggcgagggagccaacctaaggatacgcactgggtgggctgctgtggtgcagggttccgggggctgtacggcctggggcagcatcttggggcaccgggctcaccaggctgcatccacaggtgcccttcttcctgttcattgtcttcgtggtgtacacactactgcccttcagcatgcggggcgctgtcgccgttggggccgtctccactgcctcccacctcctggtgctcggttctttgatgggaggcttcacgacacccagtgtccgggtggggctgcaggtgagggatgggctaaggcctctgggggaggttttgtggtctgggtctaagggcagatgggggggccccctctgggtgaatcagacccgaaggccccggagccgtgtttgcagacagcccgctccaaggccgggccctctccacgtctgctcggaagctgggctccaggcgtggccctgccctctggcctttgctttgctggcctagggctcccctgtggctcagccctcagggtcgtgacctcaggccttgcctcccggttcagggacactgtcgtggggtgagtcccattgccgaagtttcccctcgaaggccctgtcctggcggggcttgggggagtaccccagggcactgccttgtgtctttgtgttgaatgagtgaatcttctgttcataattattgggtgtttagagaagtgttttccctctgaccaggcctggtgtcctctccaggcagaggctggccctccacacagccagctgggcgctgtgccctctgggactgcccttagcacgcatgcccctcgggccctgggctctgtgaggtggtctcttctgcctccctgggtgtcctggtcttgcagtcctagtgactcactaggagactagctcctgtctgggcctcagtttccctgtctgccccaggaagagtgccattctcagtgggttgaaggtcagggaatgggaagaggtgccatcactagtccgcctaggggtctggtgcctcgggagggcatgggccaggccacataatgagccaaacccctgtctacccgcagctgctggccaacgcagtcatcttcctgtgtgggaacctgacaggcgccttccacaagcaccaaatgcaggatgcatcccgggacctcttcacctacactgtgaagtgcatccagatccgccggaagctgcgcatcgagaagcgccagcaggtgggacccggcccccactcctcaccctgtacacccctgtcctcttcctctttctgttggacatgagacactcatgctgccatgtgcatgaggtgcatggccagacacccatgcaggtctcacctcggtgagtcctgggctccagcagggtaaccatagcctgctgacccttgactctgccttcctctgtgtggtttcagtcctgggcacgcagtccccaggtggcggcagagaaggccgtagtggctccaggctgctcacccatcagcttagcctccccagctgaaagagccctttctcagtcaccctgagtcccgggggctgctgggtacatgggaggtagatgctcagttgggttggtggaggacaagagcagagatggggacattgtgggcagggcaggcaggcagggaggatgggggccaaggaggccaggtctgggacctgatgctaccctgcctgtcccccgctggtccctccgccggtcgctttgctggtccctctgctggtctgcccacccacacccaggagaacctgctgctgtcagtgcttccggcccacatctccatgggcatgaagctggccatcatcgaacggctcaaggagcatggtgaccgtcgctgcatgcctgacaacaacttccacagcctctacgtcaagaggcaccagaatgtcaggtgggcggtgagacgtgtgattagcatatcccggggactgggcccaccgttccaccggcccccaggcggcctccccgccctatctggaggctcagacccctgcccatatagggattgggagagggccccatggttccaggtcaatctggggccctccctgggtctcagaacccccacttgtgggcaggatcagatcagggctcttaaattagagaatttagattggtattgaaagatctagcacacccccaaaactgtaaaactgtgtgagcaaacatagcttttgtgaaggaaggggtcctttcagatggttctcaagggagcacaggacccccagaatgaggtaaagccccccggccttggtgcccaagagtgccccggttctgacacctgtctttggctgatttgcagggaaccacaaaattttacttgaatgcactgacctgagtttgtcaagtgtcgatgtgggtgctgggtgccgttgagtgtagatgtgggtcccaggagaccccctgggtcctctgcctgcagcgccagcattacatccaaggctggcccgctgccttttctctgggctgtggatggtccatgagcttaaggactttggagcagggagaactggtgcagatgccagctcttggcttcccagcaagtctcaatgtcctggagcctcagtttgtgcatctgtagaacgggagttcggcaagggctgtgtgaggactcgatgcctgtaaagtgctgggtgtggaggttggcttgagggagcatggagctgcctctcaccagggagttctgaagtctgtgctgtggtgaacattggatgggaaaggggaaggctccgggctgcctgcaggagtgcccttcggctgtctgcagtgatagggcctggggtggggccgagggctcctgtaggctctgaattttgctcccttctgctttaacgtgaagcccctggaactttaatctgtctctatggctgggcgcgactctcccatccctatggaggctgaggaaggacgggggtgaatgggctcaggcctggcaggctcctggggctccagacagtcctgcgtgctcctgctgctgctgtctaggagcctggctctgacactccctcccaccctgccccatccccagcatcctctatgcggacatcgtgggcttcacgcagctggccagcgactgttctcccaaggagctggtggtggtgctgaatgagctctttggcaagttcgaccagatcgccaaggtgagcccgctggcctacaatgggcaaagccaggcccctcccctgcctattacaccccaggggtgtcctgtgttcagtgccctgctggaattgggatggggaggcgtggctgaattttagcatccagccagcaaacacaagcgccccatggtaaaggtgggggtgagtggtggccacggcagccaggatcctgtgcccccagagtgacaggccagtggggagactgacagtgcacactgggcgatatactggggctctggagctcagatgggggctcaggaggaggagatggctccatcctcactggcaaggctagcagcctccactcacctggtagctcaggaggactgtggaatgcccagcctcacccagaagctgcttttcacaggatctgaggtgaatcatgtgcacattaaacttttacgttttgtttgtttgtttttgtgagatggagtctcattctgtcatgaaggctggagtgcagtggtgccatcactgctcactgcagcctcgacctgccaggctcaagtgattctcatgcctcagcctcctgagtagctgggactacaggcatgcaccaccacgcctggctaattttttttttttttttttttttttttttttgtagcaatggggtttcgccatgtaggccaggctggtctcgaactcctgacctcaagtgatcatcccctcttggcctcccaagtgctgggattacagccgtgggccaccactcctggcccaccttaaagtctgagatacttgaaaaaatatctgaggaggaaagggaacagtgttcttagcagggggaacagcatagacaaagcctggagtagaaagaaagccccgtgtgtccatggggtgaacgtgctcagtgtgcgtgtgaggtgtgagggggacaagggcacaaggtctgacatgcaaggacatgagggctgaggcgaggaccaggatgctgaggagctcgacacgtgccaggcagggagggggtaaggcaagcaacatttccattaatggaccaaatggtcctgagagagggtaagtgccaggccctgagtctgggtggggccttgggcctacatggtatgtgctttgaagaagagaagtgtgggggccaggaagcctggggagcacctagcccagcctcaggtgatcagggcaggcttcctggaagtggtggccattaaatagagactgaagtgtaagatttaaggtggtggctccctatcctggctgtacatgcaaatcacatggaaagcttaaataaaacaccagtgcccatgtccacacccatcagagttctgttttgttttgtttttgagacagagtcttgctctgtcacctagcgtgaagtgcaatggcataatcatggctcacagcagcctcaacctgcctgggctcaggcgtcctcccacttcagcctcccgagcagctgtgactacaggtgtgcaccacaaccccaggctaatttttttgtattttgtggagatggggttttgctacattgtctaggctggtcttgaattcctacgctcaagtgatccacccacctcagcctcccaaagtgctaggattacaggcatgcaccatcatgccctgccccatcagaattttttttttttttttgagacggagtcttgctctgttgcccaggctggagtgcagtggtgtgatctctgctcactgcaagctccgcctcccgggttcacaccattctgcctcaccctcctgagtagctgggactacaggcgcctgccaccacgcctggctaattttttgtatttttagtagagacggggtttcaccatgttagccaggatggtcttgatttcctgaccttgtgatccgcctgcctcgacctctcaaagtgctgggattacaggtgtgagccactgcgcctggccagaatttgtaagagccccgcaggtgtctctgtgggcagagaatcaatggtctagaacttgatgggaggcaagttatgacttcacactgggaagaatttttcatgatcagagctactcaatcccaggagggactggaagtgggctccctgttgcagggggtaaccaagcttctacaggattgcactggtcagggatgctctggaaaactgtcccatcctggggattgtggactgagtgactcagagggcctgtctagcctggaaccttccttcaggatctgcatggcttctgaaccagcagctgggagccccagaggttgggcctcatgtgaggcaggggccctgggctcgggccacagctggccctttccttaccataaagcccacacagagggtcccttgcccttggacacagtcttccctctccagcaggcccaggaggcgtggccctattgcacagatggggaaagggaggccatggcaggtgccagcttcccagtcagagcatctaatctctgggcagagttgggcagggctggtcccagccttttctcctatcaacaatcagattccagaagtgtaagctctttccaagagccagggcctcctctctcagaggcatacaagtcacacctccgcacaagacccagctccccaactccaagtgtcagagacacaggggactgtcagccggtggagcagccgggggaatgtggcctctcctacatccccgagagctgggctgggtccatgggcagtcctgtgcctggtccgagaggagatcagggtagctggagcctagtatccaggtctgccaagattgcctgctttctaggaaagccactgtcctcttgtccttgcagtcactcagaactcactcagcatgtgccagatggggcccagcaggaaacatctggcacacgctgaaactgagtaattggcggagggcacaggccagggatggttcccaggctgggcaggtgcaggggacctggaggcagggctggtgctgtgtctggggcctgggtctgtgatggctgatgggcacagagtagggacgaggacaggataggctcgagtcctggagacagatggtgttccaggagcaggcctagccctcgtgagcctgcaggacagagccctcttagctccctcctggccagaagccagagcacagggccacctgctggggcgtccagtccgagggtcagctgagtggggtagagggtggggaggggagagcagaggcagatggcgctccttcttcagctcccctaaagccccacccatgaggcctcggtgcatgtggccacccttgctgagggcttaaggagggtctggtgacgcagcagtgcctcagatgaacctcagagggctctcgggggtccccttctctccctgggccttttcctatgagtccccaccctcgccgctctcctactctgccctccccattccctctttcctctctgcattgatatctttcctccccacccctaagccctgtcagcacagccactgcttgggtgaccttgaaacctgctgatcccttggtgtgtacacctgtgacctctgtcctcaccccccactcctgcagcctcctggctacctcgcagtctcttccccagctttgtggccccagggctcctgtgagtccttgagtactcttcctgcagagactggcatggccggctctctcggttcctcggggtcttagctcaaatgtttctgattccaactctgagaggtcttcgctggcctcctgggaacatcacttccctcctgtgccttggttttctccttagcccccatcacagctgggcacactgttttgtttttgtctttctagtttagtgcccacccctcctgcctacgtgcaagctccgtggctgcagagatttgcatctattttgttcactgctgtgtccccttgcctggcctggggccgggtactcagtaggcactcaaacattacctgggcaaatgaaaccaaggcagacccctccccgaggcccttaggaacactcgtgttcacctctggtgcttgcctgggggttcctcctggggaggcccccatgtcgctaagccttttgccaggcccagaagtggctcctggtgactggaccccaggaggcaagcccccagccaggagagagccgggtgcaccctggagggtggggggaggcggctgtcgtgagaggtcccagccccacatcttccagtgaccaggcccttccctcaccctgcaggccaacgagtgcatgcgaatcaagatcctcggcgactgctactactgtgtatcgggcctgcccgtgtcgctgcctacccacgcccggaactgcgtgaagatggggctggacatgtgccaggccatcaagtaggtcctgggcgggcccaggccctgggtcctgccctgtggcggaccctcctgggcatggtaggggatgtgtcattctcatgtcacagatggggaaactgaggctcggggggttgggggtgaaagcagcttgcctggaccacccagcctgctggaggattcagacttcatctgtttgcgtccctccctgtggtgctgcctgtgactctgagtcccagaaagcatctccggagctcagagctgggtgatgtgaggtcagggcggtgggctccgctcccctcaaggatggggtcttagcaacagcaggaggcttcagggtaggcagccgggggacctttccgacccaggggtcgactccaaggctccatctgcttcctgctccccagtgaagatcctggagtcaggcttggtcctgaggctgcggggagaccccccgctcccctctaggtcccctgtggctctgagcagctggggtctgtgggaggaatttgccgagagggctgtatggggagagggccaggcaggcccagccaggctgaccagtgtggggcagagcagggcccggtggggctgcccgtcctggatatcaggccgtgttccagcctgtccttctcctgaagtcctgccataggtggctggtgttgccagaaagaagcctgggcagagctggaggggcctggcctgcaccactgcctgtgtgacctcaggctggcccctgccctgtaacaggcggaggttgtacaggccctcgtgcacctctggttccgaccctggaaggcttggtcagtgcccagccttgttggtggacacgtggtgccgcttgtcatggggactcaggctctgttcctgtcttgggcgtcccctgtgccccaaggaagctctcccgtggggctctgctcctgtgctcagtggtgctaacaactcccagggcccctgcttcccagggattgggggggcatagaaggtgcccatggtctgggcccctgtgttccttgtctatgctaggttttgggtggttcctggaccccctctgtagacgccagtaaactttatttatttatttatttatttgagatggagtctcgctgtgttgcccagagcgtatctcggttcaccacaacctctgcctcctgggttcaagcgattctcctgtctcagcctcccaagtagctgggactacaggcgcctgctaccatgcccggctaatttttatatttttagtagagtcagggttttgccatattggccagggtggtctcaaaactcctgacctcaggtgatctgcctgcctcagcctcccacagtgctgggattacaggcatgagccaccacgcccggcccccagtaaacttttaaaacccagccggtgctgctgggcagggaaaacacccagtgatggtattttaggtccatcaacatgggctgtagtgactagttggaattcctcctccctcctacctagtccttgaggtcagctgtggccacacctacttgtatgctcctggggatggggcactcagctcttcctgaagaggcagccccatcaggctgagaacattctctcccgtgggctgagcgcctgccctgttcccacatgctgccctgtacccaccccacacctgggagcttcagcctgtcccagggcttaggcagggctggggtgactgggccactctgccccaggcaggtgcgggaggccacgggcgtggacatcaacatgcgtgtgggcatacactcggggaatgtgctgtgcggggtcatcgggctgcgcaagtggcagtatgacgtgtggtcccacgacgtgtccctggccaaccggatggaggcagccggagtacccgggtgaggctgggctgggtagccgcagggacagaggcctggggctggctgtggatggagggttctgcggttgggggtgggggtcaggtgtggagggagagatgaatgtagaaaagctggcctggggcccaggcctgcctgctgtgcatccctgggtggatgccagccctctctgggccccaaggctctgcctgacttgggtctcccgtagccgggtgcacatcacggaggccacgctaaagcacctggacaaggcgtacgaggtggaggatgggcacgggcagcagcgggacccctacctcaaggagatgaacatccgcacctacctggtcatcgacccccgggtacgagggctcagaggccgcagctgggggggacccggagggactggaggggccctggagagcctggcccgcaccttggaggaaacccccatgtggagggagaggtggggaagggccctgcctgggcaggagtgagctccctgttcccaggcacattctaggcaccgagtagccactccctgggcctccattcttttctatgtgatgggaaaacagcgcctgcctcaccagaaggagctaatccgagcgaggccttcacagtgcgcctggcacgtcacagcattctatgtattctcgtcactcttggtttattttccatgaggtggttattaatctcccagatttccgagagacacactttcaaaacaagctttggatgaaactcaggaaagcagggagtggcctgggagccactgggcaggagcctctgagtgtcctgggtgctgactgcaggtcccctggtggctccaggaccccacggtggaggaggcagttccactgccctggccaggtggggcccgccttggcctgtctgctgcccgaccctggcctgcttcagggagtccggcacgtctggccacttctctgtgaatcatccatgaggcttgctcaagaatgtcggcccagccagcgagtggctcctttcactctggtcctcgctatgccttggggctggcccgactgccagaccctgggggaccaagggctggcctgtgagcctccctacacatctgctggcctcactctgcccccagcacccccagtgctgcaccatccatactgagccagcatctgggaaaacccctgccggtacctgtgtcccattccaccaccctcctagaatagggccctgatcctcgccctgtaccccggccctggccccgcccctccaagccccttcccctgctctcctgtccccctcaccccacctggtctcctcctgcccttccactaccagggtggtggcacccgcaggtccctgcactcaaaacactcctccctccgtccctaatgcccttgcttgtttatccattttccttgtctgttgtcagcatgagtgccatgccggggggctcctcctctccaggctgcagccccccaccccgtgctgagatggatgcccggctcgaagctagtgctcagatagcccttgaatgagtgaagcagagctcctcacccaggaggctccaaaatccacccccgcaggctgccaggttgagcagggaaaactgcaggctgcctgggccgacttgaatttcagataactgtaaatcatgtttcgcataagtgttgcacgcagtactcaactggacagttgtatttatttggcggccccaccttccccccaccccaccctaccccaccctcttttttgtttaacttcatttatccacgcagttgaaagttcaaaaggtgtgaagggcgtgttgtagaaagactccttgtccccagccggcacatctcagaggggacctgtgcagtgagtttcttgtattccctccacagggattttcacggacaccgagtgaactgcgagtccccccacccttcttgctaaggtggcagtggcgggccccatggtcgcgcaccctgcatttttcctaccttggggacatccctcctgtgtgcagggtgctcgtctgtagctcctgtttttgacatggaggtggattttctgcactggcttctatgggctctgccttacgccattgctgtgtggcctggctggggcaaggtgggctcctgtggactggatggtggctgcagctgcccccgtggggctcataccaatgccatgcccattccgagtaggcatgtgtttgctcagaactgaggaatctgatttttgtttctgctttttatttgtttctcagggggctggggtgtctggattctctgctaagtgccagtgaccttgaacttgagtggctaggtgctgtgtaggcagagttgtcacaatggaggaggggacagagaaacgcatccacccccaccaggttgctcaccacagggccagcgcgctcttcctgctctgggacactcaccgaggctgaattccacgtggctcccgtggctaggcagatagatgcagctaagggcttaaaaatgtttctatcccaaaaggaggtgagaggtttccaggaaagttgcgctgtggcttatcgagtgcttgtagctgtggaggcttgtccatggggtgtttgtggggccctaggagacaggacttccggaggcagaggaatgtttcaggggctaaggagagactcagcagacctggagcccgcttaagctcatctctggtggccttgtgggtctttctcttccctgtcccttttccagtcctgtctggccaccccaactggcgaaggcctcaggcatgtgttcttggatcggggtgggggtctcaggtctgtggcatgagtgggggcgggtagggctggccgtgcattcagggcctggctgaactactgagtgtccagcatgaggctgggtctcgcaggcacgtggcaggtcatggccctgtgccatctgcggttgggggccacacaagtggaactccctggatcgctgccctgggggttggctggcctctggcttggccatttctcggggcggctagcacccctcttcctcctggctgcttctcacaggaaggagcgaatgcgtgccggccacacgtgggagggcagggtaggatgcctgtggctttcccaggcgcctcctggggaggagctgatgggtgctcaggccccagggactctctggttccaaggagcctcaagggcagtggggcctcgggcttgggacccaagccctgcctctctccctggggaggcagagcagggtatggggttgggtacagtggtgggtacttgctagcctcttctcggcctggggaaaggccagggcaccgagctgggaccccatatggcctggcctcagagtgcaccaggctttggctggctagtccccaagggcagccgctctccaaggggactgcgggttgagcaacttctttgctgtttattttttcacgggctcagggcggcgtcacacttcagggagagctggatggggatggcggccccttcctgggccgagaggggaggaggtggcacaggcccatgtccatgtctgcccgcagagccagcagccacccccgcccagccaacacctccccaggcccaagggggacgcggccctgaagatgcgggcgtcagtgcgcatgacccggtacctcgagtcctggggggcggcacggccctttgcacatctcaaccaccgtgagagcgtgagcagtggtgagacccacgtccccaacgggcggaggcctaaggtaggtccccctcccacccagaaagccagggattggggccccaagccaggagcaggagagtgagtgctgaagatctcctgccctctcagcctcactgtccccatctgtaaaatggccacagcctctgccccacctcctggggctggagaggaagcaaagacatttggagaatactccgggatggccaggaacatccctgtcctccttgatgcctggaccacgctcagctatagtcaggatgtctgtggcttggacgaccccgacaccttgtcctgtgtatcaagtgccggaggggctgaacctggcccagcagtggccttcagggcccagtgttctggggaatgactgtatcctttcctcccttccctttcagagcgttccccagcgccaccgccggaccccagacaggtgcgtgccctgccctcctggccaagtcctgctgcagccacctcctccaagcaggctgccctgagcagcctctgtccagtgggcagctccgcaaggcccagcccaggacctctgtgaagttggccagggcttgggtcaaagcatttctaggaaccagagtttcctggcccctggcccttctgcatggccacatgatggagcacctgttgtatggtgccctggcggggtcagtggagtcggtggaagggaaggagatgactgtgggtgtcctggtccccatgccaatcccgggggtgtagccgaagatgccttagactggggctcgggtctgccctctgcctctggcttggctgctggccagctgggccatgctgggccatgctgggtcctcactcgccctctctgggcctcagtgggacccacccattccttcaccatcccacctgaggttctgggaccccactggtgtctacgtccacaccctcctctgggagagttggaggtggtcgctgtgctgatgcagttgtgggtatctgattccagaagcatgtcccccaaggggcggtcggaggatgactcgtacgatgacgagatgctgtcagccattgaggggctcagctccacgaggtgaggtctgagacctctgtccacccccctctcctctccccctcggataggggtcccctatatgggcaggcccatctcataccccatgcctggtggcccagccttgttccttccccctgcctgctgccacctcctgagggaatggaccccttcccagcccccatctcaccgcagctgcccccgcccacaggccctgctgctccaagtccgatgacttctacacctttgggtccatcttcctggagaagggctttgagcgcgaggtgagggcccccagcagcctcctccgcagagggacggggtctccaggcctggggtagggtgggggacgcaggtcatggggcagcctgcgttcccaagaccggctaggggaggggcactgagaatccacagctgctcctgggcggggggcaggggcggggccagtgcagcaccagcctggctctctcctgtcggcttcattccatgacccaaatgcaccatcagagatttgcctccatgggtctgcaagacttttgctccgtccagtgccaaagccttagcagatcctggcatggatgcctcaggctgatggcaccggccttgcaatgagacgagaacccaaagctcagttatcagtgtcctgtcttgctgctatgagctttttgtactgataacgaccatttctttactgagcgccttctgggttctggcagcatcctcggcacccacattatataatttaaccgtcacaacggccagcccaggggtgctgctataccacttcacagagaggaaactggctctcagaggcttcagagcctgtccccagcttcaggtgtggctccctgagttgggggcttcctaggtgaggtcacgaggaaacctgctggccaagtgacctggcagggtgtggccagtgtggccagggccgccgagcctgctttccttccctgcagcaggaacccttctggggctgtgatcctgcgatggtgcctgggtgggagtgggggtggggggcgggatggtctccctacctgccagcttcttggtttgaggtgaggacagccccggaagctcagacttggctcctgtccatgtacttggggccatgagctctgcagggaccttggaaagagagagacgggtggtgtagggcaggggaaggcattgtcttcaaacaggaaaaagctgagaatggaaacaggcgaaacttaccaagtgtaacatcacctggaactgaaggagggtgggaaggttttaattattttaaaaatagagatggggactcactatgttgcccaggctggtctcaaactactgggctcaagtgaacctccttcctcggctcccaaagtgctgggattacaagcgtgagccactgtcccagcagggaggtgttttttttaaagctgattcactggaggcagggtgggcaagtggcactgctggtggccacccctcacagtccctgctgcccccagtaccgcctggcacccatcccccgggcccgccacgactttgcctgcgccagcctgatcttcgtctgcatcctgctcgtccatgtcctgctcatgcccaggtcagttgcagggaggggtgtgggggtccggcctgctgggatccaggctggaaggtgactatgaacctgcaaggagctgtgtgatttgggctggaaggggtcggctgctggggtcctagcaactggaccaggggctgtggcagcacaccttgagttaccaacacttctttttaatagaatgtgtgttttctgccacaggcctccctactccctaacgtctctctcctcagccacctgtcatatgtgtggcctgcatgcattttggttgacagcccttgccttaggtgtttggagtgctaggaggatagactctgaaaactgtaggcgccatcctttttctcttatatatagggaaattggggcacagaggattaatgatttatccaaaactcactgagattcatgcttctggctctagggccctgctggtggggtatagggatgagggtgaagtcagagggaagggggatctaaggtcagctacttggtgctttctagaagagcagttaggccgaagcatcgaccaggattgtggttttggctatgcttactaaagacataatagggaggctgtgcgtggtgactcacgcctgtaatcccagcactttgggaggctgcggggcgaatcactcgaggtcaggggtttgagaccagcctggccaacatggtgaaaccccgtctctaccaaaaatacaaaaattagctgggcgtggtggctggcgcctgtaatcccagctactcgggaggctgaggcaggagaatggtgtgaacctgggaggcggaggttgcagtgagccgagatcatgccactgcactccagcctgggtgacagagtgagactccatctcaaaaaaaaaaaaaaaaaaaaacgaagaaagatgtaatagggaatgcgtttttacagttttttctgagtctaaaggctgcagagacattgctcatttcttatacacttggaccaaaaagagtgatatggtcttgacctcaatcttcataatctagctgggccctgtgtattgcccatggggcagctgggccacagttttcacaggtcccctttgctgtgggcagaataccaggtagggtggggacaggtggctgtgagccagaggattggtgggggcggtggtcctgggcagaaggccctaggcagaactgaggttcttccctcctctccaggacggcggcactgggtgtgtccttcgggctggtggcctgtgtactggggctggtgctgggcctgtgctttgccaccaagttctcggtaagtggggagctctggccccgcgggccctccctccctgcctcaggacacctgcctaggagccctccctggtgagcttggctgggctgagcccctgctctggtcaaggttgggtgcccatctctcctgcctcggggacaatttcctgagtgccctggaggcttctcccgaggatacccctccccaggctgccagcgaccagccctccttgcccaggctgttggctctgggtgacttgaccctgttaccccacagaggtgctgcccagctcgggggacgctctgcactatctctgagagggtggagacacagcccctgctgaggctgaccctggccgtcctgaccatcggcagcctgctcactgtggccatcatcaacctggtgggtcccgtggtggggaggcaggcctccggggtagagggagacctccagatttgggacgcctacaatgtggccttaaaagctgcctctggttgtcctctcccccgagctcagcaggtggatgtggggggttcccctttgtacactcagggctcctcacctttgggttctcaaatgcggactttgggggcccagagagcttgtgtggccagctccaggaggatgtgatgccttgagccaccacatcctgacatcccattcattcttcacggcccttctgagactcagtttcccctctcagggatgaacccttgctcctctgctggggctcagaggctcagtgctgcagaaagcagcctgcgggacacttgtccacctctctcccagaccccgcatcctgggctagggcctctccctcacctctctgccccggaccctgggtttttggaatgtgttcctgtgaccacctccctcagggtcacctggggaggctgaaaatgttggtcccggcccctccccctggctgctgccgctctgggggtgtctgcaccctcatctcctagcccctctgccctctgaacctgacggctgctgtggggagtcggcacggcggcttgagccgtgctcagagctgatcccaacgtgaaacctcaggctgctgtgatactcatggttgcctggggcccaccttgcatggcttgggcaggttccagcgttcttgtccctggactgatggggacctgtctcctctacagcccctgatgcctttccaagttccagagctgcctgttggcaatgagacaggcctactggccgcgagcagcaagacaagagccctgtgtgagcccctcccggtgagtgcgccgggcccggctccgtggcctcattcagagtggggctgctgctgccagaggtgtagtttggaccctcaaagcatgggtgctgacccctgagagcacagcatgtggaggctgagaacagcctctcctgcagagctgggaaagcagggtcccatgggcccagctggcctctggcccaagggagtgaggagtcctggccttggcaggcttggtctaggctgtgtgggtaggcagggagtgaggggagcatctggccttgtgcgggtctctggtcagggcacttcctctctgacaggcctgtcactattttatacacgagtatgcacagctcagggaggggcttcattaggtgatggcgccagggcaggcccagacaggctgatggcagaggtcaaggtctctgcctggagggccttgcagacagcagactctgcagcttcagaggctggcagtgcagtgagctgggctctaaaaggcagaaaggttctgggacaagggacagcatgggaaaaagccgaggcgtgtgttgggagcagtggggttttgggtgggttgtctcctggagagggagtggggagaactgggagatggagccgggggcaggcttgatccagactgggaggtgcaggaatgccggggtgtgaagagcgtggtctttattctgggggctggggagccacagcacaatcttgagcagggcagaggtcctttgggagggtaagctcacaaaaactcagggaggcagcttggatgcatgcacgctccggccttgaccttgactgtggtctgttgtatccacagcacctgcatactgttttttcccgtttaaatgagctcacttcataagaaataaaactacagtgaaaacaacactggacactcttaggtctcattgttttttgttgagaagggaggtggtaaggcaagagcgtgatgctgaggtgcagggagtggggctctgtggtatgtgctgggctggaggcgagagtacgtggtggggtggccctgtcctgagtgacaccctgccccctcagtactacacctgcagctgtgtcctgggcttcatcgcctgctcggtcttcctgaggatgagcctggagccaaaggttgtgctgctgacagtggccctggtggcctacctggtgctcttcaacctctccccatgctggcagtgggactgctgcggccaaggcctgggcaacctcaccaagcccaacggcaccaccaggtggggtcccgcccgtccccgtccccatccccatggtggcctgttcatctggtgcctgcctgctcgcaccaaggggcttctgtgttcatggggagtgggcaccttgcagggcggggctggcagatggtgtccagcgctcagagccgaggaattcactggcaggttctttaggaagtccctagtctgacagaggccaccgggtcatcctgtttccaggccagtctagtggtggggagggaaggaggctgcagctctagctctgtgcctctcaaggccacatcctgggtctcgagttctggaaggagcagttgcttccatctcttcctggacaggtggtgatacctgaggctggcagggctggggtgtgtggttctaggggttctgggtcccaagtggattaaggagcggacctggcccccctaaacaaaaaagctggaagcctgggacccagaacagaacctgctcctgtgggtctgccccgcacctgcaaaatccttatgccacccatccaccccaccccgcctgccactgaccctggctcaccagttctgggctcccttcaggccctcatcacagctgattctggtcattcaggggtgagatcagaggtcttccccacaagggctcccccatctccctgcccacctctcccctcccccctttataataccccaaagttgtcttgttttctacccaccccattagaatcaattttcccctttcccctgggtgtgcgtggcacctggagagcatgactcagtgggttggagtggtgagtgctgggagtgacttgggcctcccttcgcattcagtggcacccctagctgttcctggaaggacctgaagaccatgaccaatttctacctggtcctgttctacatcaccctgcttacactctccagacaggtaaggaggctggccccccccccccccccaagctctgcccacttttcctcacctccatctggagatggggtggggtggggtgggctcaggtggagtagcagaaaagactagagtcctgccaggaaagcactggtcccctggccagaggctccaaggacgccagggacagacagacctggctaggagatgcacagcagcacggctcccacttgcctgtggacggggcgcttatgttgctgccgtttgcagattgactattactgccgcttggactgcctatggaagaagaagttcaagaaggagcacgaggagtttgagaccatggagaacgtgaaccgccttcttctggagaacgtcctgccagcccacgtggctgcccactttatcggtgacaagttaaacgaggtgtgctgagaaggggctggggcgggggcagggaggcggacggtccaggcgcagtccgtagggtgaaggtgtggagctggagtgcatgctgagcttggcttcctcaggtcctggcctcaccgggatgcccaggcgacaatgtatggcatcagctgtgagagcatgggcctggggtcagggacctgggctcaagtgtggtcgtgaacaaggcaccttgctttcccaggccttggtcttcacctctgtaaaatgggctgacagcccttccccacaggcttgtggcgaggatgaaatgtgtgttcaggggtttgtgatctggacattctgtgttgatgaaaatgtctgtacttctactagttatgtgtagcaagttttctagataggaaacggaggcccagtgagaatgaagacctcccgttaccctccactactgcagaggttctcaccccatgtgtacatggaaacacccatggtgcttctgtgagcaaaaaccaacctgagcctgaccctccctggccacccaattcttaatcacccggtaacagccaccagatgaaactcatgcagcccgcccccctccccactccccgaaagctgggctgggcagttcagcagtgccatctgctcctgaggccttgccactcttcctgtcccctcaagaggtgaagtggtgtaaggtccggtttcttcccatccaggactggtaccatcagtcctatgactgcgtctgtgtcatgtttgcctccgtgccggacttcaaagtgttctacacagagtgcgatgtcaacaaagaagggctggagtgcctacgcctgctcaatgagatcattgccgacttcgacgaggtacagcctctagcccagccttgcgcagcagcccccacccatgctggagagggaagggcggtggcacctgccatcctaaaacccaatttaaaaatgtgacacagagctgggcaaggtggtctataatcccagcactttgggaggtcaaggtgggtggatcacttgaggctaggagttcgagaccagcctggccaacatggcgaaaccctgtctctactgaaaatacaaaaaattacaagctgggcttggggcgcacacctgtaatcccagctacttgggaggctgaggcaggagaatcgcttgaacctgggaggcggttgcagtgagctgggatcacgccactgcactacagcctgggcaacagtacacaactctgtctcaaacaacaacaacaacaacaaaaaaaaaacgacgtggcccgcctggggggaacatacattacacagaatataaaggtacacatttctttttcattctgttttatgcagcaaataattcgttggcatcttctctgtgatgggcagcttgctaagattagactcaggccccttagcttcatttccaactaagcccacgctatcaaccaagccaaacagaggaaatcagtttgggttgaattctttgctggagacaaagaatctacattcctgtgtagataatgctgtgtttgctctgtgcagacacagatggaagggaagagggagacagtgtggggacggagacaacaggaagagcaggagccgccaagggccatgtcctcaccatgcttagtcatgcgctcagctggcagggcagccatgagacgtgtgtgcgagaggcggcgatgggtggagggaaccccacacccttcctgagaagcaggggcagcctggctgcggctatggagtggcggctgcttcaccgcttccttcttgcctgcagctcctactgaagcccaagttcagcggcgtggagaagatcaagaccatcggcagcacgtacatggcagctgcagggctcagcgtcgcctcagggcacgagaaccaggtactcaagcccaagaggtgaaattcagctgactgtcctcacttaaaggtgacctgtcaccttacttaaagggtgtgcccttatctcgtcctgggggaggggcacaggtacttgtagggctcagccaggattttagtggtggggatcctcaacaagagtggcgcgccagggcccactaggtctgggggctctggagatgcaaggatctgacccacagcctgtcccgcaggagctggagcggcagcatgcccacattggtgtcatggtggagttcagcatcgccctgatgagtaagctggacggcatcaacaggcactccttcaactccttccgcctccgcgtcggtgagcccgggtgatggagcggggtggggagcccctgcctctaggccagtccacccagtctgtgtggcacagctgcacctcgccatctattatgaaaggttttagaaatccctctgatctgacaagctcagcagttggacaattattctgatgttttgcatatagttagcatatagttaccattgagagttttaataatacgtaactacagaaaatggagaaccaaaattattgtctgtaaacaggtgaccttaaaatatagagaaggaagcaaagttactaaacctaaatagttgcgggcctgcgctcaaagccaggcctttgttaatacttttaacaaagtattctttgttaaaagagagattagcagatacaagtgttaacatcaaacccagacttctgagtctgaaaaagagctggccggggaatgccctcctcataccccaagcccgactgcaacgcagtgactctgggaagcaacccagccttcactctagttgttttgtccccgcatcctgtgctggggtatggattctctggcctcctctaagggcagaagcttgaggctttgcctgcacgcttgggtaactgtaaacatcatcttcaggcataaaccatgggcctgtgattgctggagtgattggggcccgaaaacctcagtatgacatctggggaaacactgtcaatgtggccagccgaatggaaagcactggagaacttgggaaaatccaggtaaagacctattggggaagcagttgactaaggggaaaagatcttccccagaggtgaggggacttggacttaagagctgctgtctcacccagcagccatggttctagctatcacactctccagggaaggcagactgcatgcctgggcagtctctatctgtccctaccatgacaggtgttcttcccgcctctgaagacaacaggtctctcttccttttcaggttaccgaggagacctgcaccatcctccagggcctcgggtactcttgtgaatgccgtggcctgatcaacgtcaaaggcaaaggcgagctgaggacttactttgtctgtacggacactgccaagtttcaggggctggggctgaactgagggctcctgctggattccgaaaaggccgggaagccagtctccttccctgaagcaagcccaggagaagactctccgccccacgccaatcccaaaggcatgcagatggctgtgcatgttggcttctttggacctgcactggaggatttctcagacacatgcaccagattctggctcgaagcagccactgagccataatgcgcaggggaggccagaagctctgtgcctggtctgtaacagtttccaggccagctggagaatgttcactggttcggggctgactttgagatctttgttccctgaggtgccaggcaggcaactttagcacatgatgaaaacagacttccacctcagtggcctgtgggcacgcacaagtgaggtctgtttttctagacaccaagggggagtaagctgagctgtctagcacggattggagactccctctccctggtgggcctggcaatgacagcatttctcacagaggcattctggtaaatgaagctgaaaggggtgttttacatctgtaaacggtttcaaacaggtagagagaaaaacaccacaattaacactgttactttttgccttgtctggcatgtttgttttaaatgaatacattaatggggtttttatccttttgaatgacttttcagacactagacataaatctcttccctccagtgtatgctctgcctttttaaccactgacatgtaaggaggactactgtctagcatcagcttatggggtcagctggctgtggggatagagtcctgaggaatgtggtcacagcaagaaggcggggagcagcagagccttgcctttgaatgaggcagcttgtgaggcaagcattctggagagaggtgctttgaaagtaaggtgcggcctttcacctcttccttgattactcacacatctttgcgttctcccctgccgtccttcaactgtatcttacttttcttaccagaaaggaatggagtctgtttagagacaacttggacaacctgtgagtgcatctcttctttcctttagtcttcacagctaactctggagagcttcaaaactagaaggatctactccgcatgggtgcatgcagaggctcctggatctgggaagcccgccccctcacaaatgctgagccgttcttgctctgaaactgcgtgagtcaaggcaaatgcaaaaagccaggttttggggatgtgtcttactgtgcttcaacttcccaaggaattgaaagtcaacctaactgtaacaacagggtgagaaatgaccaaactgcccgtgactttttctgaatggacttcataaccggaagacttaaccggtggcctcatcaccagagcatcgccaggatttctaatgcactcagtttccctacatagcagggattcttagctaggtgtccccatgaaccccgtaaagttctacacaaagtcttgcatacaggagcctttacaagatgattatacagggttgcagattgggtgactgaccagacttgttggggtcctgggatgagttgccccgggctgcaaattaagagtacagctaagtgcgggggtggcggtggagggaacgaaaattgaacctgtctgcctgtgctgtgtcgtgtggctttatcagcccgaggaagggcaggtgtattctaatttgcacaaaggtgctgggtagactagtggcagctctcatgtgctgcacataagtggaatcagtatgaatagaagaacttgctgtataaaggaatttcatggcaacaatgctggtaagggcaattagcctcgcttaagttgccttttttacacaccaaaactttttacatgaagggctggtttcacatgaatactatactgaaatctgtgctctcaagatctagcagtgaccagggctgcccggcgggggctctcctggcaagtcaggaaggtttctgttgctaatataacatagaaacacattagtgcactgggcctctctgaggtcagcatatttgtactcttggaatatttgtttttttcttcagtaacaacagaaaccccagttgggagtttaacaaataactgactaccactcactcatgcatttttatttccaattaaagcaaagcactgtgctgtgctcagataataatagtttgtaagtaaaagtttttagttttcagtgttcaggttatagaatataactgaccataaaaattacctgcaggtattttctttttatgaacttgtttttaaattaccaagtaattactggtgtcattttgttttatgacagacacacgtatctaacaaacaaacaaacagtgaccttctccatgggtcaaggacttccttacaatttctcctgagttaacttttgtgaaaataatacctaaggttttctggcttattgaggaaatttcctaacaaacaaacaaacaaacaaacagaagagaagatcattaaccactgtatactttgtgtatataataggtcagtgtaaagaaatatgatttgaggtggtgcatgcaagtaactagggtttattctatataatgaatatttatagatctgtaacatttgtttcaaaatgctgtttcatttttataaagtaccagtgtttagctgctttttatacattaaattagcaatttgaaaaactcaaa

SEQ ID NO: 2 - DNA, 51620 bp, homo sapien
ccggagcaggaagtggtgggtcccagatgtgactcactctcattaggtagcgtggaaggagccttcggatgggtgagcctgggcgcgtctgaggaagggcaggcgggggccgggccacctccctgcagaccctggccggctgctggggccggggaggagagccaggtaaaagtggggtgtggggatggagtgtcaccagtcctagtggcttgagccccaggggacggggctggacagtgggtgggaagcagctcacccggcagcctggcctgggagtgcactgggcagggtctgggctgggggagtaggaggctccgtgagcctactgggcagatggctgggagaggcggcccttcaacaggggaccagaggcgacttgtcactggcttttcccagcctgacaagaaagtgcggttgtgaccacaatgttgtacatgtggcccgttccatctggaattctaggaaccacagactctcaaatgtccacactccttctcgagggtgtaattttacatttacaagcacacatgacttttctcaatgaaaggaaacattggcaagggaaggccaattcaactaattttatgaaattataaaacaaaaaacaaacaaaacctctgaaaggagtgcatgcctgagggtcaggaaaccaagatgcctccaaaaagcagactggtttccctttccctgcaatgcctctcctctatcccccagggaccgcatgggccatctggagtgggtctttctggagcgggtctttctggagctttctatgcattcatctaaacatgtatgtacagagggtgaggcagttgcctcttctctgtgtcatcacatcagggtcaagggcagggacatctgtggctgggggcttcctccctggacagccattgctggagaggagaagggagggccgggagggggaagccaagggcctggaggtaggggccaggccgagccacctacgacaggggccctgggtccaaggcctggacgggcagccagtgtgggctggatgtgtctggaagggctatgtgaccgtgggccttcactagcatcaccatcttgcagatggaggctgtgaggggtggactcaacaccagtgtttctcggagtgaggaccgggctgcccagggaatcacagggggttctagcctgtgtgtggaaggacatgtttaaaatctaagtatttgtcttggggaaaaaaaaatctgtaaataggacattgccattgtgacatcatgactgtattattgtcttagacgaggccaaaggagacataaaagtgacaagccagccaatttgagaggaagtgccaagtgccagccctgcaggaagtgcaggaggctgagggggctgcagctcaggaaggcgtttgctgcacagccttcgcttcccacccagttgtctgtcttcccacctccagaggccctgggttcccaaggtttacctgcagctcctggtgcagagtgtgggggaggggaggctctcagattgttccagattctgtggccctcagaagggcaggtgtgagctttgtggaggtagcgtctccggtggtattttctttctttcttatttttatttattattattttttgagatggagtctctgtctgttgcccaggctggagtgcagtagtgaccctcttgggtcactgtaacctttgcctcctgggcttaagccgtcctcctccctcagcctcctgagtagctgggactacagttaccacaccactacacctggctaatttttgtatttttagtaaagtaaagacggggtttcaccatggtggccaggctggtctcaaactcctgacctcaaatgatccatccacctcgacctcccaaagtgctgggattataggtgtgagccaccacccccagcctcttttctttttttaagacaggatctcactctgtcacccatgctggagtgcagtggagtggtcatagctcactgcaaccttgaactcatgagctcaggcaatcttaccgcctcagccttctaaagtgctgggattacaagcatgagcccgacgcccagccttctagtgctgttttcatgaaagaagcagctgtttactagcagctgttttccgggtgatgagggtgaaggtggtattgcagaacatctttgccaggagtaggtctgggaggtttgcttgagggcatggaggttggtgtagggagagctcaggaagtagaaattgggctggagtgtggcctctggacaggccacaggagagtctcaaaaggagaaaagatcccagcactttgggaggccaaggtgggcggatcacttgagcccaggggttcgagaccagcctgggcaacatggcgaaaccccgtctctacaaaaaatacaaaaattagctgggtgtggtggcgtgtgcctgtagtcccagctacttgggaggctaaggcaggaggatcctttgagcccaggagttggaggctgtagtgagccatgactgtgccactgcactccatccagcctgggtgacggaatgagaccctgtctcaaaataaataaatttttcaaaagagacaaaagtccgactcgatggttgcactggggacatgcaccccactctacaatttatagctactgatttgacccattccccacctcctccaccgctccatatggtaggtgccgttcacctccagtttacagaggagacagggcccagaggggctgtctatcctgaataacccagcagatcggtcctcaggcatccagttgtgcccaccacagtgtggtctgctgccacatttgagcctggagtcagggccagcacactgtgtctttggcgagatcagatggggcaaagctgagaacaggggctctggttggtgctgagggattcgtgggggtggtccttactgggaagatgaggaattgggctcctgtgagggcctcccctaccctgccctgtgcctggacttcgctgggcccactgcgggagagaagaggtaggacgaggccttctgcgtggctgggccagggtgggctgtctcacgtgctttcctggatgtgtggttgggtcatgccttagccaggaggtaggtggtttgtccactccctgtcattgcaagggtcacagaggtggctgggtggcctgaccctcagcaccgctccttgcctccctcctgctgtcctggaggaagaggatgtggacttgttcatactgaggtgacacagcatcctctctcctacctctcccactgcccagggagagagcagcgcttaactccgctggagaaagttccaggcctttgagggagcatgcagagtgggtcacagtggatgcctgtatcagagcagcccattctagcctttcatggggtgggagggaggcatttactacttttaaaagacacgtgtggccaggcaccgtggttcacacctgcagtcccagcgctttgggaggctgaagtgggcggatcacttgagctcaagagtttgagaccagcctaggcaacatggcacaaccccaactctaggaaaaaattacaaatattagccagcatggtggtgcatgcctgtagtcccagctacgcaggaggattgcttgagcccaggaggtggaggctgcagtgagctgagatcatgccactgtactccactctgggtaatagagcaagaccctgtctcagatagatagatagatagatagatagatagatagatagatagatagatagataaacagatagatagacacatgtagccaagagctggaagttagttggaactggagaatgagaccacatctgaacaccagccagagaagcaggggtcttccagaggttatccggtccagcatcccgcctccaggcagaactggactgtgctctggtccggcggcacgggttccagctctgttctgcctcctcccaacagcgtgtgagatgagcaagggatggagcctgtttcttgcacctgtgaaatggggttggtaaaggtaccccctgcatcaggtggttgtaaagattaaatgagctgatgcacactctgctcctggcacagcggaggcgctacctaagtggaagtgtgcgtcactgtcggtgtcgtgaggctctcgtctgccttgtcctgccttgggtgggagattggacctccctgggggagcctgggtggtgcaggcaggcagggctgagctgagcagcctccctgtcacctacatccctcggactaatcattttctttctgttcctcagtttccttatctgtaaaatggggctgatggcagtgctcaggtaggttaatgggagggctagctgggtgaaaacccttcccaggcacctgacactggtgtgcccaggctgagaggttgccacacagtggctggagcccctccagctgtgtgtgccggggatcccgccagccccctcatctgtgcagtggccccctccccaccaaggacagtagcatggtgtcacccggcttgccataggcatctggcctgacctgctgctgcacacggatttctcaactggccacatcatggctagggcattgcccaaccctgagtgcagacctgcttggccgaagcaccctccgttccctgctctcccctggaagcctggaagcctttctagaagggaccagtgttggggtggagaagggttgggtggtgagtctgggaccagagctccgttcctctttggtggcctttttccttggaatacctaggaccgtgctttcctgccgccaccagctctctcattttttattttgacagggtctggctctgttgcccaggctggggtacagtggcctggtcatagctcacagcagcctcgacctcctgggctcaagagagcctcccaccttcacctcccaaagtgctggggttacaggcatgagccaccacacccagcctgtcctctttgatgggccaatttcacaggcattttctgagcacagcacagcccagtgctgccgagatgaacctgagcagactctgtgttaggatggggtggggacggctgaccttgggcctgggagaaggtcctccctgttctagagcacagccatctggtcagtttctcctggcttgtgaggccctcccagaccgggcctgacttccctccccactcccgccacttgtcaccgttatccactctgggccagcccccctgacattttcactcccagcttggctatctcctgcccctttcctgcccttggcccccacagttcccactgcctggggtgttctccccaactgctgcttggctgtgtcctccaggtgagggcctccttcaggggtcctccagcatggggagaagaggggacccagccggctctgctcttgagctgttcactcagccgcctcccccacccccttggccttgctttgagcccctgtcttgctgtggctggggcccaagggacgccgggataccctcttctcaccatcctccctttatgtccctgtttcccccaccaccgctgaccactggaagggtgaacccacgactgggcctgagggaaagggaagccaagggaggtctttggtgcaggtacccccgtttccagttgtgtttcctggccccaggctctgcccattcttgcctcctcccagggtatcaggacatgctggaggtgacagggcagaaggaatggccatgaccctcagacacaggcctgtaccctggggaggagctcatgtccctggaaggcagaaggtacctgcagtgggtgatgccaccacccgccttctagggcccggatccctggaggccgctggacccagggcaccctgatcggggtgctttgtcttccaggccccaggctcttcctgccctccagggtgcccttcctgcccctgtaactggccagctttcctcatgctggcctctgaaacaacctagtcacactgcctcgggcgcagctgactgagatcattgcttcctggagtcaccgactggctgggcagggagaagcctagcaccctgggatgcagctgctctgcacagatggggaaactgaggccctgggagctctgcagccttggggctggtgtgtcttcctggtgggagctatgttggagttgcaggccctgccccattcacaagcacatcccttggcctcactcatgggagggatgtggactgggggccagtcttgtgggcttgtcttggtcctgctgctgttcttgggagtctgggtctccctcctcctgggcatctccttgctggagcaggggtccagggtgctggtgtaggctgggatgagacctccttctgtgtgtgtgcgcatctggctcctgggcccccaggcctgtctccggtaccccctctggctgggaggaggagcccttttaagctgtctgaaggagctattttaaggggggcctgggctaatgggaagtggcctcttttgatatacccagactccctggagacactgctccatcccctgggggcagggaccagcacttctgtatccctttgtcaggctcagctctcctgggcatgggtacaagagcagagctgccctttgggggcaggcaggaagtgaggctgagaccctggtactgaagtgtgtccccagtgcctgtttatgtcacgatttctcaggctgctcaccctggtgtctggagctgactggggtggccctgggtgatggtacttgctgtgtgcccacatctacgtgaagcccctgagaactggtattgttaccagtccattctgtgggttaggaaactgaggcccagagaggttcaggaacatgcccaaggtcacacagctcctccaaggtggagctgagattcacggcctgctgttggatctcagagcttgtgctcccaaccaccatgctttcctgccttccccttctgccctgccctggcatttcttttcctcacttggctcatttatgacaatctgccctcctcctggcatgagctcctcaggggacccatgcctgtcctgtgtgttgtagcagcagcagtgcctgcacctgctgcaggcaggagctcagtggcacttggcagatgggggtctggtggcaccaacatgtggcctctttcctggtggcaggatgggcccctttccatgtgcaatgggggtttatgaggttttatagctatgaggtcacgtcactgctagaataggccatggagcgaagagaagggtttcatccccattgctcaggtgagaaactgagtcctgagaggcaggtcacctgccagagctcatgcagttgggactggggattccagggttggggcccagtccttagctgccagttcagtgctggctgtaccccccagtctgtggagtatggggtttgatgtccctgtgtaccaggcagggttagatcctgggagctgggtagtgcatggctggacgtgcagccaggtagtgacaggtcagggtagcctgtgcttctgtgggaggtgcaggccctggggaagctaaggaacaaggccagactgggaggggaccaggcagccttcctggaagaggtggcagctcaggttggccctgaagggcgggcaggagttcgccaggcagaaggcatttggaggtgaggaggtgggcgtgtttttagattgccggagctggagatgaaagggtggggcttgtttggggaaccgaaatcagccaaatcacgcagccacagaggaggccagggaagctctagaggcgttttttggccaaggggtgagcagagatcgggcttggtgttttggggggtcctgggtaggtgtgggaggcctcgtttcgggggccacggggctcctgtggcgtgagactccatcacagtgacctcctgtgcccaatagctccgtaagggcagggtttgtggaaggaaggagtgagtgaccgggaatggggtcttgggctccagggacagccatggcgagggctcttctgtgtgggtgcaggtccggaagctggggctgtggttggtgggggaggctgggtggggctgcttcccctgcagaagggctcttccgggctcttggcgcggtttcacgaggcagtcccctctgtgtctctcgtgccccctaggcctggatcgggttctcaggaagcctcggggaagtgagggtcttagcgccttggtttggggctgggcactttgcgagctggtgtcatggaggccaaactgggggcctccgggccaggtggtggcttcaggcttgctgttgggacaggaggccaggctaggcagtgacgcctcactgagctccatgggtggcacgctgagctctgggtgggagaggcctctcccactgacctctctgtgactggcttctctgctggtctgagtgggaagggccctcagagaccctggaggcctttcctgccctggaggagtcaggcccagagagggtgaagaattgcccgagtcacacagctggtgacagggagagctgggactgtaccttacatcacctgacattcagcccagggctctattccctgcactgtggcagatggggaaactgaggcaggtgggccactcctctgcattccatgggagaggcaggaaattcgacaggagccctttgccgggttggcacttggagctgttttcctgcttgcttttccccagggcaaggtgcttcccagctgaaaactgcctagggagggtgctgttgagaccgtgagcacctgagagccctggtctttcactcaggcctctcctctcacctccacctgggaggctaagctcagggtaggtgtcctggcccacaggcaggtgcatgggctggtcccgccagcacctgcaaccttcttgtgcgcagctagcccaacctggggtcaggtggaggtgggtaagtggggcccccagcattaaagagcttcataggctgtcaattaaggggaggctagtgggctctacaggccacagctacccagggagccttcttaggggaggagttcctaggtgggtctcagagactccaaagtcagcatttcccaaaggaggttcattagggattcagtgcaaaaagagtctcattaacaaaaggatggggaaatgttcagctgtgtttaattcagcaggcttctttcctgcaggacttgtcagagcctttaatttgctaatgtgtatgggggatctttaaatggggaagaggagaatgttgagctcagcaccacctgtgaccagaaatggctgtggatgtgtgtggctgcgcatatgtagcttttcatgttcctgcttttgggcagaatgcacctcaggaaatgcagatagagagttaatctgcactgggccttggagaggggcaggcctgggttcgaatcctagctccactgtttcctagcaggtgggctttgggcttggaggttaaggtcccagagtcagttgccctggctagaaatggagcgagagcacccccgcccaccacccttgggtagcccagcccattcatggttctctgcaggttctcagaggagacagccagccaggtggtttttggcccagtttgtctcccggctgggctcagaggtcgcagagcttgtcttgaggcctgggtactgtgccgtctcctgccagcaagcctggaaggcttgggccgaggacccgactctgcctgggatgtcacctgtgagctccctccagctcagaccaatatcaccacctccctctgacctgggttgggtgggacggctgctcccttgaggcccaactaggccctacatggaggggtcagcccacactgccactcatggccctggaaagccggcctgagttgctgaccaatgttctctctacctcccagaggctgggcactcacagaccagagtaggggccgggcctcctcacccccagtgctgctggctcatggcagggttgaggggtggggacaggtgccgggccctctcacttctgcaccccatggccctttctgacatcttggtgatgcatgccatctagactggggcattttgcgccaggggtggctcagtacactgcttgcatcacccccaactgaatcctttcaaaatcctgtggggtaggactgttattctccttttgttaaaatgaggaaactgaggcacagagagtctgttgggtcatgtaatgtattatttgttttaatatttaatttatgaatgagtgagatggagtcttgctctgtttctcaggctggaatgtggtggcacaatcatcacttgctgcagccttaacctgaactcaggaggtcctcctgcctcagcctcccaagtttgctggctctacaggcatgtaccactgtgcctggcttagttttaaaatttttttagagatagcatctcactacgttgcccaggctgatcttgaacttttggtcttaagcagtcctcctgcctcggcctcctgaatagctgacatcacaggcatgagccaatgtacctggctaggtcatgcaatttaggtgtgatgggcctgggattcatttttcctattacacatttttttttttttagtcacataagcaacacactgacaataggaatagaaactgaaaatgagaagtgggatttatccactctgtgaaatcaagagttttggtttttctgtgtcctggttccatccttatttgttccaggcattgccatgtggatttttcacaggataccctgtgacgttagtttcaagtggcctcaggctttttgacagctgcatagtattccattgtaaggttgtatcataatttatttcaccagttcgtgactgataggtatattattttcaatcttttgttattacaaataatgcttcaatgactaatcttgtacttatgtcatttcatacttgtgacaatgtttgtacgatacattactcaaagtggatttggtaggtcaaaaagcaaatatgtccttcagtgactttgttaggtattattaaactaccttcactgggggttgtactgactcacattcctgcccaggagggaatgtgagtgactgtttccaacacagacttatgagacttatgaaatgctttgatctttgcccatcagataggtaaaaatggttttccatggtagttttgtttttgttttaaagatggggattttgctatgttacccaggctagactcaaacttctgggctcaagtgatccgcctgcctcagcctcccaagtagctgggactataggtgcatcccattgtgcctggctaccatggtaggttttttttttttttttttttgaggcggagtcttgctctgtcacccaggctggagtgcagtggcacaatttcagctcactgcaagctctgcctcccagtttcacgccattatcctgcctcagcctcctgagtagctgggactacaggcacccgccaccacgcccagctaattttttgtatttttagtagagatggggtttcaccgtgttagccaggatggtctcgatctcctgacctcatgatccgcccacctcggcctcccaaagtgctgggattacaggcttgagccactgcgcccggcctaccatggtagttttaatttgtgtttcattacgagtgtggttagcttccttccaaatgctgtgggttggttgtgggttgcttttttttttgagacagagcttcactctgtcaccgaggctggtttacaggcatgatcttggctcactgcaatctctgcctcctgagttcaagcgattctcgtgccccagcctcccaagtagctgggattataagcatgcacctccacacctggctagtttttgtattttttaatagaaatggggtttcaccatgttgctcaggctggtctcgaactcctggcctcatgtgatctgcctgactcagcctacctaagtgccaagattacaggcgtgagccaccatgcctggcccttccaaatgtttaacagtcatttagattgccttttctatgaactttctcttcctagtcttctcccatttttctattgggctattggtcttcttttttatttgtgggagctctttacatatgaaggagattagctccttgtgatatgaatcacaaatgttcccctaatctgtcacgtgtcttttgattgtgcttttggtaatttttaccatgtatattttgttttttcatgtaactgaacttatcagtctcttctttatggcttttgggttttgtgtcaggataaaaaggccatattcactacagcatcatatgagaattctcccatagtttcttgctctctttggaaataatcctggtataagttgtgaatccagcttgcttgctgtcttgtggatgctgcctttctaaacagaatactagcagcctaagagttaagactcaaatgcacatctttcctgtccccttcaggacagtggttactctgggttgcatctgtccatgggcccgtgaaatggatctctctctctctctctttttttttttttttttggcagagcctcactctgttgcccaggctggagtacagtggtgcaatcccatctcactgcaacctctgcctcccgagttcaagtgattctcatgcctcagcctcgcgagtagctgggactataggcacgcaccaccaccttgggctcatttttctatttttagtggagacagggtttcaccatgttggccaggctggtctcgaactcctggcctcaagctatccacctgccttggcctcccaaagtgctgggattacaggtgtgagccaccgtgccaggtctatggagctcttcttgtacctcccagccccattcatgtcttgctgtcactgctggacccactgtcagggtggggccctaggcaatgtgggaagtcagccagcaccctcttgtcctgggcaggacgagaaccacgtgaggtcccccatggccagctggacttcccctttctgttcttccttatcaagggaaaagggatcagaaatctgttaaatgtgggcgtgaaagtgtttcccttctgaagggggaagctagtgagggcagtgcttaaagaaatgaatcactaattgaatttcaagcaagagtgaggaaaaagtagggctatttctccaacaggggagcctggttgattgcttagtcagttgataaacactgttgcctgaacacccgctgtgtgcagagacctagcagataccctggttggggcagtgctaaggattctgccttctgggagctcatttcctgagcagaattccctcttggtacctggcctggcacctggtgcacagaaggcatgagtgcacatctgctgaaggtatgcatggacgaactctccctgggcagtcactaactagcattagaatgggcatgagacatagtggacctggattcattatcattggcagctggagccttgggggtgcattttaaaggggagattgatcagggtaatgcttaaccaaatgaatgaagtggaggggaggatttcaggaacaaagaacactttttttttcctttttttgttgtgtctctggcagtttttggtaacaagatgatgctggcctcatagaatgagttagggaggagtccctcttcctcaatttttttgaatagtttctgtgggaatggtactagctcttcttcgtatatctggtagaattcagctgtgaatccatcaggtcctggccttttattggtaggctatttattactaattcaatttaggagctcattattgatctgttcagggaatcagtttcttcctggctcagtcatgggaggattgtgtgtccaggaatttatccatctcttaggttttctagtttttgtgtgtagaggtgtttgtagtagtttctgatggttgttttttatttctgtggggtcattagtaacattcctgtagtcatttctaattgtgtttatctggatcttttctcttttcttctttattagtttagctagtggcctattttattaattttttcaaaaaaaaaaaaaacaactcctggattcgttgatcttttgaatggttttttgtgtcttgattttcttcagttcagctctgatttttgtcatttcttgtcttctgctaggcttggggttgatttgtttttgcttctctagttctttcagttgtgaagttaggttgttaatttgagatctttctaagtttttgattcggacatttagtgctatgaatttccctcttaacactaccctagctgtgtcccaaagaatcaaagaacaactttctacccatccctagaagtagctctctggggccaggtgagaacatcagccttttccctcagtttttctccagttgcctatccttttcattaactgattcagcctccacccccagcccagtcccaactaggccagagctggcctcagaggtctgacacagcccctcctttgcaagaggggaaactaaggcacaggtcaggcaagagacttgtttagggtcacagagcaggttggtggctaagctgggactggaatgcaggtttctggtccccaagactgatcagcagcctgtgccctgggagtgggaacaaagtcagcctgggggtgcttgggggctatggagggggagcagggttggctgggggctcatgccgttggggaatttcagtctttgcccctgctcggcaactttgcaggctgttaggagatgcggtggggggcctgcccactctgcacctgcctctccctggaccccatggttgggctctgaccctgcagggcttcctctgacactaagcgtgccttcttggccatttgccaatttgtcttgcaagcttggcctgttggacatcatgacctgtgcagacaacctggttatcagcacccccagtttactgatgaggaaactgaggccagagtgtgcaggcgactgctattcacaggccattggtagcagagctgggattgggtgcaggcatcctcccagaggccaagcgtagctgggagaggtgtgctgtggcaggtaaggcaggtgcggcaggtgcagcaggtgcttcccgatcttgagggaggccctttcttcatctgccctcatcctttggctgtggcgccctcctccctcacccatgccactctgcgtgcttccccaccctgggcctggtgcaggattctggctggctaccctgctcggccattcacgtcacaggtgtttgccaggaccagctgcgggacacacacacacagatcccaccacgacagtttggcaagtcaggggccgggatattggacccacactgggaaggcctcttggtacgggaggtgcctgggagagcagggcatgggggccacagggctctggggacagagagggctgggcttggggggatgggaccttgaataccagcaacattgaatccctgggtcccacgaggtttactgggggcctcttaggtcaggcttgcactgggcagagcagctgctggcttcatggagctcagggtctcttgagagagttcgcgttaatatgagattcccttggcaaatgaaaaatctgcctgagacccagcctaccaggggagccgagagccggcaggaccgggggcgcgagacggagcatgcaggttttttgtctggagctgcgtggcgtcccaggcagggtgtgggcccaggggcctgagcggggcctcttccctggaggcttccatgcccccctcactccaaggtccccacttcccctttttgaggccaacagggccccaaagcagccctgagcctgctgaggctcggccggaggaagccaggctgtggtttctgccaacagtggccgggtgaggaagtgggtgtcctgtctgcatctgtgaaagctgggggtttctagaacatactccctgcaagggctgtggttgtgctggggacgctgccagatcagctggactggggaaggattttccgagcacccagggacctggccctgcagttgcagtatggagtcccagcctcttgcttccagcaggtcttctggctggagaacctggacccagccagccagtatttgataaggagaccacagtcctgccttagttgcagggaagggataagggagtgtgtgtggggttgctcagcaggcttcctggaggaggaggccctcctggagttcgctggcctgaacttgctcctgcaaggcagagggaagtaattcgatggggtcccctgagctccccacctcttggtcatagctctgtcctgggccttgtttgtagggaagaggttggtgttttgggatattatgggatttctggatgccagccccatgagggtgatagggaccccggatatcctgtgtttaaatcccagctttttttttttttccagacaggattctctatcacccaggcaggagtgcagtggcatgatcatagttcagtgaatgtagccttgacctcctgggttcaagcaattctcctgcctcagcctctcaagtagcttggactacaagtgtatgccaccaagcctggctaattaaaaaaaaaataaacaactttttatagcgatggggtctcattatattgcccaggctggtcttaaactcctgaactcctggctggggctgggctgtggagcctggaaagtgtgatggagggatgagcggggctgtcccctccaagacccagctcctttctggaggactggctgtgtctgcaggggcacactcttacctctgtgctgactgagacccttccagttgactctgaacccactgaggggccacgggtgcttgttagacgcaacaaactgggtccagaagcctgatgtggactttgagactcttggttgcgagtgacagaagctcaaactggctgaagctgaaaggggcattgcttgtcccatgtggttaaatatctagaggttgcgtaagcttcaggcacagctggctcctgtgaccccaggaggtgttgccaggcccctgtcttgtgccagttctccatgttggtgtcagtttcaggtgggctctctccttgtggtggctcgggtgtctgctgacagtgtcaggtcaccgacaaaagagaacgcctctttccctgggtcagcagaagtccaaagagagccttcacttcccaggcctgtggcattagggcttgagagggactttgctgattggccacactggggtcatgtggcccatgaaccagggtactgagggtcgggagaagggacattgcacagcagaagcatgaaataccctcctcccatgtggacaccttccccacaggggagtcccctcttctttaaaatggggctgctcaccctgctctaccactggggtggtcgtgaggtgagagtaagtaagggaccccctgggactgcagtctcctcaggaagaggggacgtgatacctctgctcttggtggctttggagttcagacaggacccatgcggtgtgggtgagggagtgacagggaggcagggcttgccttgagctaagggaggactcagggccagaaaaagtgcccagtgatggaggagactgccctgagaggtggtgagcccccccgtcatggaggtgtgcaagctaacagcagggtcattcaatccgggagccctggattggatgtgtctcagacatggcagaaggattcctttatccagggccttgtggggaggccctccctgttgccaactgagtcgagggctggtggaagaactggtgattggacaggaggccttgaaaacttcccgggcagggggtgcctctgggtctggctttggcggggtggctcctagggaggagctggcctggggacatgccactggtggtgggttcctgggccccctctggagatgtgatcagtgagttcattccccccaggcctcacggtgcccggcttgttccaggagctggccaaggtccctggatgggctgaagtccttgtgcccatgttgtccactgtccacctgcgcctgcctgccgttgcccagaaagtggcctaatgagttccttgagaggcgtgtttaccaggcctgtgttcccacgggcccttgggactctctcagctggttcctccctttcctcgtggtggcccagcctggctttgtgtgcctggagcgggtgtgtccacacactctcacacttgcactttcatgcacgtgtctggccgcctcagctctggtctgggggctttgcctgggagatggagcagttgtggggagtggcctcagccctctggtgtttggagcttctcagcctggctgtcctggtgcactggtgtttggcagcaggccaggggtcaggtgggtttgctgtttgtgtgctgtggcatctggctcagtcccccgggatgtcagtgggcaagcctgggctgaggagccctggtttgggagctggcagacctgggtttgcatcccagcctatctgtccaccagctccagcagtgtgatcttaggcaaccactctgcctctctgggctcagttctgcatctgtgagacggtggtgccaccttggcctttgccggttgtgaagatgagagtgaatgtgtgtgcaggtgccgggggtgacaggaactgctgctgttattactgcaaagggctattttggggtggggggtgctgaattgaatgtatctcaggcagggaccttatcttgtttggcaacccccacagcacagggttggatgtatatggttagcacacagtaaatgcctcttgtggaatacagactgattagtttagatttagaggttgtaaactagtgggctgcaaatattgaaaaatttgaaatcagctgccaacatttaaaaatcatcatttcacgtagaaatcagatttgtagcctgtctgagcactttgggcactttgacagctcttggccacatccctgaagggttgcccctcgttgaacaggtgccaaggcctgtagacacctgcccaggcactgaaggggatggggcccccagcaaatgaccccttcctccttccccaaatatgtggttttaccattttgttcctggtgtgtggtgatggaataggaacttccagaggaaatagagcctgaggtggctcctggaattgaggtggccccagggatctcatactagcaggtgctggttggggacttggggaccctggatatcctgctttttgcctttgggggctgggggctcacctggccatcttccctccgctggaccagctgtcctttgaagcttctgctggcggctggccatttgtgccctgtgactgctctgagctgtcccgtgtctcacagcctccctgccctctgcctctggccctgtcctggttccaatctgaggcaggcctggaggacactgggggcgtggactcttggtccagggttgcccccttcatgcaaggttcctgctgggaatatggcagctgtccccaagtccccattgctgtgtgagggattgcacaattttggtaattttcctacctttggagactgatggggatccctggggagagagagcccagactagcacacaggagatataagggctggcaggcgtaggtgtgattcacaagcctttttggaatcaatgaatatgctaaggaatgaatggtgctggggaaagaaggaacccatatgggctgagcatggcaagtgtggggtcagctcacagtgttgtcacaaaggggccactgtgtccctctctgagactcagctcccttttctgtgaagcacggcttgcagacatctgaaatttgtatcagtgggctatttaaaaaaataaacaagatttttgctgggtacagtgcctgaaagcttaacacttcggaggctgaggagggtagattgcttgagcttaggagttcaagactagcctgggcaacatggcaaaaccccatctctacaaaaaatacatggcctactgcagcctaagtctccccaggctcaggtaattctcccaccttagcctcctgcatagctgaggtcccagctacgcaggagtctgaggtgggaggattgcctgagccctgggaggttgaggctgcagcgggctatgatcacaccacttcactccagcctgggtgacagagtgagaccccatctcaaaaaaaaaaaaaaaaagagagaaagaaaatgcagttttatgtggagcttactgtaacacagatgcagggggctaccctggctgaagcaggcatgggggcctgagcctatgcccccccaccttcccttcccattgggggcctgatgcatgtccccaaatctcagggctcctaggatcacagcttggaaccctctgtgcctggaggcatgagactcactggaaaatgaagtgaatgtggtgattgcaccaggaccatggggacagagcagagcctggaaacagacaccatctgtgtgtgatcacctgacttatgacagaggtggctccactgaaattcagagagaggatggccggttcactgaacactgctgggataattcaatatccatatggaaaaaagaagtgaaccttgatccctacctcacaccatacccagagtgaatcgaaatggatcagagacctttgtgtgaaagcaaaacagtgaaacatgcagagcagagctggcctgtagaagcttctgtgatgatagacattctctgtttatctgtgctgtccagtacagcagctgcatgtagccattgggtatttgatttttttttttttttgagatggagtctcactctgttgcccaggctggatgcaatggcacgatctcagcttactgcaaaatttgcctcccaggttcaagccattctcctgcctcagcttcccgagtagctgggattacaggcacacgtcaccatgtccggctaatttttgtatttttagtggaggggggttttaccacgtgggccaggctggtctcgaactcctgacctcaggtgatctgcctgcctcgacctctcaaagtgctgggattacaggcatgagccaccatgcctggctggctgttgagtatttgaaatgtggctagtgtgactgagggactgaatttctaattttattttaaattaatttaaatttaatttaaaatagtggctgggcatggtggctcatgcctgttatcccagcactttgggaggctgaggcaggaggattgcttgagcccacgagttcaaaaccaccctgggcagcagggggagacgcccatctctacaaaagagaatttaaaaattagctgcacacctgtagtcccagctacgtaggaggctgaggtgggagaatcacttgagcccgggagtttgaggctgcagtgagctatgatcgtgccactgcactccagcctgggcaacagagtgaggccctgtcttgaaaaaaaaaaaaaaaagcagccacatgtggctagtggctactatcttggacagtgcagtcctagaagacacaggggactagctttacgacaacaggacataaaagttaggttagggttaagatggataaatcagacttggtgctgtgaggcctgggccccgctctccctgatgctagccctcccctgacttggaccattaggtcaagttcctgtctgcttcgtctccgcagagctgaggaactgcgtgtggagtcagcccagtctggatgcacaggaggatgctggcggcacagtgagtgaggcctggtgccagagctgtgcggaccccttgttggccatggagcagcaggcccagaggccctctccccagccctgcttgcctgcctcggagaggacagaggcctaggcccacgggggagggtgttggcagacagatgccctccaggccctggggcctccttaacggccccttaacgacacgcgtgccaagggtggaggatgccagccaaggggcgctacttcctcaacgagggcgaggagggccctgaccaagatgcgctctacgagaagtaccagctcaccagccagcatgggccgctgctgctcacgctcctgctggtggccgccactgcctgcgtggccctcatcatcattgccttcagccagggggtgagtgagggcagcccctgggcttcacgtctcggccccaaccttggccaagctgctatcttctcttagcctcttctgtaaaatgcttatcttctgtaaaatgctatgcttttttgtttgttttgttttgtttttgggtttttttgagacagagtctcactctgtcacccaggctggagtacagtggcatgatctcggctcactgcaacctccatctcctgggctcaagcaattctcctgcctcagccttccgagtagctaggatgacaggtgcatgccaccatgcccagctaatttttgtatttttagtagagatgggctttcaccatgttggccaggctggtctcgaactcccaacctcaggtgatccacctgcttcggccccccaaagtgctgggattatagacgtgagccaccatgcccggccaatgctctgtcttttacagcacatttagactggtagaagacaagtttctaatttaaaaaaattttttttgagactgggccttgttttgttgcccaggttggccttgaactcctgggttcaagtaatcatcctggcttatcctcctgagtagctgggagtacaagcatgcacgaccatgcccagcctctctaactttaattcagttttgaagtctggagtggtttcagacattacattaacttgtccagataaactactctcatttttctccatgatgaaattcgattctactgaagacctgggtcagggatggcctgtgtatcactgccctcacttcctgtacccatgcagacatcattaatcaatccctacattccacactgagcctgtctacattctcagaatcctccttttgttttttttttgtttgtttgtttttgtagagacatggcctcgctatgttgcccaggctggtcttgaactcctggcctcaagtaatcctcctgccttggcctcccaaagtgttgggattacaggtgtgaaccactgtgcccggcctcagaatcctttttaacacagctctccaggctggcaccacctatgtcatttccggcccaggcacttgccttggtacagccactagctgtgtttagctattgagcatttaaaaagtgggtaatgtgactgaggaactgcatttttcaaaaaaaagagttttgctcttattgcccaggctggagtgcaatggcaccaacttggctcgctgcagcctccacctcctaggttcaagtgattctcctgcctcagcctcccgagtagctgggattacaggcccatgccaccacgcctggctaatttttgtatttttagtaaagacagggtttcaccatgttggccaggctggtctcaaattcctgacctcagctgatccacctgcctcggcctcccaaagtgctgggattataggcgtgagccaccatgcccagcccattttctttttagtgaatgtaaatttgattagctgagttctagaagaacactcagaaactccagggtgtttatgtgagtgaagtggccaggacctagcagctgccagtgcagcctggatccatgtggctaccaaccacaggatgcttgctactgtgtgccagcccccagatcaggcctctgcccccacggacctcccggatgctgtgcccgtcctgtcccacctctgtgccattgcctcagctcctcccctgtgggatgacttttcctggtatccctcaccagcctctgctcactcactgagggccaggcgcagtgtcctctttgggcggctttacaaggctctgcctgtgaatgagtcctgccctgtgcttgtggtatcacctgcggctgtgcacagagcaccgtgggaacccagagaggaggcagaggcagatggagagtgatggcatcatgggcggggctgtgggtgatgctctggttcccgagatgatgggaagggggtggttgaggcagccttggcagaggtgacaaaggccagaaggtgagtgggaaccaggtgtgggagggtgcatccacacccttcacgtggaggaagctgtaagtgaggcagccggcccggcaggctttctggaggaggtggctctgcactggggaaagccgaggcattcctgtctgtttgctccacccaggacccctccagacaccaggccattctgggcatggcgttcctggtgctggcggtgtttgcggccctctctgtgctgatgtacgtcgagtgtctcctgcggcgctggctcagggccttggcgctgctcacctgggcctgcttggtggcgctgggctatgtgctggtgttcgacgcatggacaaaggcggcctgtgcgtgggagcaggtaacaggaactctggactccctgccagctgcgccttcagcagctcccatccccgtggttggtgggcatgccacaggcccttcgtgccccacgcttggctgtgtgtctaggatggccccaggctggttttgtccctgtgagttctctgcattgacagggctgaggtggggagagcatccactcggacgctgggctgcagcacttttgggtggctccagtttggttggagatgacagaacttcagctcaaagaggcaaaaaggcacaaactggttcctgtgtcaggaaaggccaaggggtcagtgctgggctccagtgggtaactggaactggggcctctggacagcttcggccccgggcacgctgtccccaggtgggggcagagaagttcacagcggctccaggctgcccaccccagttgagagagagggccctttctcaaccagctcgacctgcaggcttgagccacactggcctggctgtggtcatgggcccagtcccagagaggctgtcaccaagcggtagcacactctgaccagtgggcctgggtcctatgacagctcctgtggctggggcagggtggggtgaggcccaaggaaaccatgtggactgaaggggagggagagggcagcactcccagaggggtggacgcaggaccagccggtggggatggtggggcagctctgggggtggagggagggcgggtggggccgcattgctggaggcagctggtgagagggtgcctcagcctcccggtaatcatttctgctgagcgagaaccacgtggcaggggcaccaggtgggggctccctggatcacgcggggcatggagcctcccacctagggtggccacaggcagtggatcagcacaggaaaccatgaaccttgtggccgggcccaccctagaaacacattatggcttttcttgttcccttgtatgtctgcccacgcagggggtggcgagggagccaacctaaggatacgcactgggtgggctgctgtggtgcagggttccgggggctgtacggcctggggcagcatcttggggcaccgggctcaccaggctgcatccacaggtgcccttcttcctgttcattgtcttcgtggtgtacacactactgcccttcagcatgcggggcgctgtcgccgttggggccgtctccactgcctcccacctcctggtgctcggttctttgatgggaggcttcacgacacccagtgtccgggtggggctgcaggtgagggatgggctaaggcctctgggggaggttttgtggtctgggtctaagggcagatgggggggccccctctgggtgaatcagacccgaaggccccggagccgtgtttgcagacagcccgctccaaggccgggccctctccacgtctgctcggaagctgggctccaggcgtggccctgccctctggcctttgctttgctggcctagggctcccctgtggctcagccctcagggtcgtgacctcaggccttgcctcccggttcagggacactgtcgtggggtgagtcccattgccgaagtttcccctcgaaggccctgtcctggcggggcttgggggagtaccccagggcactgccttgtgtctttgtgttgaatgagtgaatcttctgttcataattattgggtgtttagagaagtgttttccctctgaccaggcctggtgtcctctccaggcagaggctggccctccacacagccagctgggcgctgtgccctctgggactgcccttagcacgcatgcccctcgggccctgggctctgtgaggtggtctcttctgcctccctgggtgtcctggtcttgcagtcctagtgactcactaggagactagctcctgtctgggcctcagtttccctgtctgccccaggaagagtgccattctcagtgggttgaaggtcagggaatgggaagaggtgccatcactagtccgcctaggggtctggtgcctcgggagggcatgggccaggccacataatgagccaaacccctgtctacccgcagctgctggccaacgcagtcatcttcctgtgtgggaacctgacaggcgccttccacaagcaccaaatgcaggatgcatcccgggacctcttcacctacactgtgaagtgcatccagatccgccggaagctgcgcatcgagaagcgccagcaggtgggacccggcccccactcctcaccctgtacacccctgtcctcttcctctttctgttggacatgagacactcatgctgccatgtgcatgaggtgcatggccagacacccatgcaggtctcacctcggtgagtcctgggctccagcagggtaaccatagcctgctgacccttgactctgccttcctctgtgtggtttcagtcctgggcacgcagtccccaggtggcggcagagaaggccgtagtggctccaggctgctcacccatcagcttagcctccccagctgaaagagccctttctcagtcaccctgagtcccgggggctgctgggtacatgggaggtagatgctcagttgggttggtggaggacaagagcagagatggggacattgtgggcagggcaggcaggcagggaggatgggggccaaggaggccaggtctgggacctgatgctaccctgcctgtcccccgctggtccctccgccggtcgctttgctggtccctctgctggtctgcccacccacacccaggagaacctgctgctgtcagtgcttccggcccacatctccatgggcatgaagctggccatcatcgaacggctcaaggagcatggtgaccgtcgctgcatgcctgacaacaacttccacagcctctacgtcaagaggcaccagaatgtcaggtgggcggtgagacgtgtgattagcatatcccggggactgggcccaccgttccaccggcccccaggcggcctccccgccctatctggaggctcagacccctgcccatatagggattgggagagggccccatggttccaggtcaatctggggccctccctgggtctcagaacccccacttgtgggcaggatcagatcagggctcttaaattagagaatttagattggtattgaaagatctagcacacccccaaaactgtaaaactgtgtgagcaaacatagcttttgtgaaggaaggggtcctttcagatggttctcaagggagcacaggacccccagaatgaggtaaagccccccggccttggtgcccaagagtgccccggttctgacacctgtctttggctgatttgcagggaaccacaaaattttacttgaatgcactgacctgagtttgtcaagtgtcgatgtgggtgctgggtgccgttgagtgtagatgtgggtcccaggagaccccctgggtcctctgcctgcagcgccagcattacatccaaggctggcccgctgccttttctctgggctgtggatggtccatgagcttaaggactttggagcagggagaactggtgcagatgccagctcttggcttcccagcaagtctcaatgtcctggagcctcagtttgtgcatctgtagaacgggagttcggcaagggctgtgtgaggactcgatgcctgtaaagtgctgggtgtggaggttggcttgagggagcatggagctgcctctcaccagggagttctgaagtctgtgctgtggtgaacattggatgggaaaggggaaggctccgggctgcctgcaggagtgcccttcggctgtctgcagtgatagggcctggggtggggccgagggctcctgtaggctctgaattttgctcccttctgctttaacgtgaagcccctggaactttaatctgtctctatggctgggcgcgactctcccatccctatggaggctgaggaaggacgggggtgaatgggctcaggcctggcaggctcctggggctccagacagtcctgcgtgctcctgctgctgctgtctaggagcctggctctgacactccctcccaccctgccccatccccagcatcctctatgcggacatcgtgggcttcacgcagctggccagcgactgttctcccaaggagctggtggtggtgctgaatgagctctttggcaagttcgaccagatcgccaaggtgagcccgctggcctacaatgggcaaagccaggcccctcccctgcctattacaccccaggggtgtcctgtgttcagtgccctgctggaattgggatggggaggcgtggctgaattttagcatccagccagcaaacacaagcgccccatggtaaaggtgggggtgagtggtggccacggcagccaggatcctgtgcccccagagtgacaggccagtggggagactgacagtgcacactgggcgatatactggggctctggagctcagatgggggctcaggaggaggagatggctccatcctcactggcaaggctagcagcctccactcacctggtagctcaggaggactgtggaatgcccagcctcacccagaagctgcttttcacaggatctgaggtgaatcatgtgcacattaaacttttacgttttgtttgtttgtttttgtgagatggagtctcattctgtcatgaaggctggagtgcagtggtgccatcactgctcactgcagcctcgacctgccaggctcaagtgattctcatgcctcagcctcctgagtagctgggactacaggcatgcaccaccacgcctggctaattttttttttttttttttttttttttttttgtagcaatggggtttcgccatgtaggccaggctggtctcgaactcctgacctcaagtgatcatcccctcttggcctcccaagtgctgggattacagccgtgggccaccactcctggcccaccttaaagtctgagatacttgaaaaaatatctgaggaggaaagggaacagtgttcttagcagggggaacagcatagacaaagcctggagtagaaagaaagccccgtgtgtccatggggtgaacgtgctcagtgtgcgtgtgaggtgtgagggggacaagggcacaaggtctgacatgcaaggacatgagggctgaggcgaggaccaggatgctgaggagctcgacacgtgccaggcagggagggggtaaggcaagcaacatttccattaatggaccaaatggtcctgagagagggtaagtgccaggccctgagtctgggtggggccttgggcctacatggtatgtgctttgaagaagagaagtgtgggggccaggaagcctggggagcacctagcccagcctcaggtgatcagggcaggcttcctggaagtggtggccattaaatagagactgaagtgtaagatttaaggtggtggctccctatcctggctgtacatgcaaatcacatggaaagcttaaataaaacaccagtgcccatgtccacacccatcagagttctgttttgttttgtttttgagacagagtcttgctctgtcacctagcgtgaagtgcaatggcataatcatggctcacagcagcctcaacctgcctgggctcaggcgtcctcccacttcagcctcccgagcagctgtgactacaggtgtgcaccacaaccccaggctaatttttttgtattttgtggagatggggttttgctacattgtctaggctggtcttgaattcctacgctcaagtgatccacccacctcagcctcccaaagtgctaggattacaggcatgcaccatcatgccctgccccatcagaattttttttttttttttgagacggagtcttgctctgttgcccaggctggagtgcagtggtgtgatctctgctcactgcaagctccgcctcccgggttcacaccattctgcctcaccctcctgagtagctgggactacaggcgcctgccaccacgcctggctaattttttgtatttttagtagagacggggtttcaccatgttagccaggatggtcttgatttcctgaccttgtgatccgcctgcctcgacctctcaaagtgctgggattacaggtgtgagccactgcgcctggccagaatttgtaagagccccgcaggtgtctctgtgggcagagaatcaatggtctagaacttgatgggaggcaagttatgacttcacactgggaagaatttttcatgatcagagctactcaatcccaggagggactggaagtgggctccctgttgcagggggtaaccaagcttctacaggattgcactggtcagggatgctctggaaaactgtcccatcctggggattgtggactgagtgactcagagggcctgtctagcctggaaccttccttcaggatctgcatggcttctgaaccagcagctgggagccccagaggttgggcctcatgtgaggcaggggccctgggctcgggccacagctggccctttccttaccataaagcccacacagagggtcccttgcccttggacacagtcttccctctccagcaggcccaggaggcgtggccctattgcacagatggggaaagggaggccatggcaggtgccagcttcccagtcagagcatctaatctctgggcagagttgggcagggctggtcccagccttttctcctatcaacaatcagattccagaagtgtaagctctttccaagagccagggcctcctctctcagaggcatacaagtcacacctccgcacaagacccagctccccaactccaagtgtcagagacacaggggactgtcagccggtggagcagccgggggaatgtggcctctcctacatccccgagagctgggctgggtccatgggcagtcctgtgcctggtccgagaggagatcagggtagctggagcctagtatccaggtctgccaagattgcctgctttctaggaaagccactgtcctcttgtccttgcagtcactcagaactcactcagcatgtgccagatggggcccagcaggaaacatctggcacacgctgaaactgagtaattggcggagggcacaggccagggatggttcccaggctgggcaggtgcaggggacctggaggcagggctggtgctgtgtctggggcctgggtctgtgatggctgatgggcacagagtagggacgaggacaggataggctcgagtcctggagacagatggtgttccaggagcaggcctagccctcgtgagcctgcaggacagagccctcttagctccctcctggccagaagccagagcacagggccacctgctggggcgtccagtccgagggtcagctgagtggggtagagggtggggaggggagagcagaggcagatggcgctccttcttcagctcccctaaagccccacccatgaggcctcggtgcatgtggccacccttgctgagggcttaaggagggtctggtgacgcagcagtgcctcagatgaacctcagagggctctcgggggtccccttctctccctgggccttttcctatgagtccccaccctcgccgctctcctactctgccctccccattccctctttcctctctgcattgatatctttcctccccacccctaagccctgtcagcacagccactgcttgggtgaccttgaaacctgctgatcccttggtgtgtacacctgtgacctctgtcctcaccccccactcctgcagcctcctggctacctcgcagtctcttccccagctttgtggccccagggctcctgtgagtccttgagtactcttcctgcagagactggcatggccggctctctcggttcctcggggtcttagctcaaatgtttctgattccaactctgagaggtcttcgctggcctcctgggaacatcacttccctcctgtgccttggttttctccttagcccccatcacagctgggcacactgttttgtttttgtctttctagtttagtgcccacccctcctgcctacgtgcaagctccgtggctgcagagatttgcatctattttgttcactgctgtgtccccttgcctggcctggggccgggtactcagtaggcactcaaacattacctgggcaaatgaaaccaaggcagacccctccccgaggcccttaggaacactcgtgttcacctctggtgcttgcctgggggttcctcctggggaggcccccatgtcgctaagccttttgccaggcccagaagtggctcctggtgactggaccccaggaggcaagcccccagccaggagagagccgggtgcaccctggagggtggggggaggcggctgtcgtgagaggtcccagccccacatcttccagtgaccaggcccttccctcaccctgcaggccaacgagtgcatgcgaatcaagatcctcggcgactgctactactgtgtatcgggcctgcccgtgtcgctgcctacccacgcccggaactgcgtgaagatggggctggacatgtgccaggccatcaagtaggtcctgggcgggcccaggccctgggtcctgccctgtggcggaccctcctgggcatggtaggggatgtgtcattctcatgtcacagatggggaaactgaggctcggggggttgggggtgaaagcagcttgcctggaccacccagcctgctggaggattcagacttcatctgtttgcgtccctccctgtggtgctgcctgtgactctgagtcccagaaagcatctccggagctcagagctgggtgatgtgaggtcagggcggtgggctccgctcccctcaaggatggggtcttagcaacagcaggaggcttcagggtaggcagccgggggacctttccgacccaggggtcgactccaaggctccatctgcttcctgctccccagtgaagatcctggagtcaggcttggtcctgaggctgcggggagaccccccgctcccctctaggtcccctgtggctctgagcagctggggtctgtgggaggaatttgccgagagggctgtatggggagagggccaggcaggcccagccaggctgaccagtgtggggcagagcagggcccggtggggctgcccgtcctggatatcaggccgtgttccagcctgtccttctcctgaagtcctgccataggtggctggtgttgccagaaagaagcctgggcagagctggaggggcctggcctgcaccactgcctgtgtgacctcaggctggcccctgccctgtaacaggcggaggttgtacaggccctcgtgcacctctggttccgaccctggaaggcttggtcagtgcccagccttgttggtggacacgtggtgccgcttgtcatggggactcaggctctgttcctgtcttgggcgtcccctgtgccccaaggaagctctcccgtggggctctgctcctgtgctcagtggtgctaacaactcccagggcccctgcttcccagggattgggggggcatagaaggtgcccatggtctgggcccctgtgttccttgtctatgctaggttttgggtggttcctggaccccctctgtagacgccagtaaactttatttatttatttatttatttgagatggagtctcgctgtgttgcccagagcgtatctcggttcaccacaacctctgcctcctgggttcaagcgattctcctgtctcagcctcccaagtagctgggactacaggcgcctgctaccatgcccggctaatttttatatttttagtagagtcagggttttgccatattggccagggtggtctcaaaactcctgacctcaggtgatctgcctgcctcagcctcccacagtgctgggattacaggcatgagccaccacgcccggcccccagtaaacttttaaaacccagccggtgctgctgggcagggaaaacacccagtgatggtattttaggtccatcaacatgggctgtagtgactagttggaattcctcctccctcctacctagtccttgaggtcagctgtggccacacctacttgtatgctcctggggatggggcactcagctcttcctgaagaggcagccccatcaggctgagaacattctctcccgtgggctgagcgcctgccctgttcccacatgctgccctgtacccaccccacacctgggagcttcagcctgtcccagggcttaggcagggctggggtgactgggccactctgccccaggcaggtgcgggaggccacgggcgtggacatcaacatgcgtgtgggcatacactcggggaatgtgctgtgcggggtcatcgggctgcgcaagtggcagtatgacgtgtggtcccacgacgtgtccctggccaaccggatggaggcagccggagtacccgggtgaggctgggctgggtagccgcagggacagaggcctggggctggctgtggatggagggttctgcggttgggggtgggggtcaggtgtggagggagagatgaatgtagaaaagctggcctggggcccaggcctgcctgctgtgcatccctgggtggatgccagccctctctgggccccaaggctctgcctgacttgggtctcccgtagccgggtgcacatcacggaggccacgctaaagcacctggacaaggcgtacgaggtggaggatgggcacgggcagcagcgggaaccctacctcaaggagatgaacatccgcacctacctggtcatcgacccccgggtacgagggctcagaggccgcagctgggggggacccggagggactggaggggccctggagagcctggcccgcaccttggaggaaacccccatgtggagggagaggtggggaagggccctgcctgggcaggagtgagctccctgttcccaggcacattctaggcaccgagtagccactccctgggcctccattcttttctatgtgatgggaaaacagcgcctgcctcaccagaaggagctaatccgagcgaggccttcacagtgcgcctggcacgtcacagcattctatgtattctcgtcactcttggtttattttccatgaggtggttattaatctcccagatttccgagagacacactttcaaaacaagctttggatgaaactcaggaaagcagggagtggcctgggagccactgggcaggagcctctgagtgtcctgggtgctgactgcaggtcccctggtggctccaggaccccacggtggaggaggcagttccactgccctggccaggtggggcccgccttggcctgtctgctgcccgaccctggcctgcttcagggagtccggcacgtctggccacttctctgtgaatcatccatgaggcttgctcaagaatgtcggcccagccagcgagtggctcctttcactctggtcctcgctatgccttggggctggcccgactgccagaccctgggggaccaagggctggcctgtgagcctccctacacatctgctggcctcactctgcccccagcacccccagtgctgcaccatccatactgagccagcatctgggaaaacccctgccggtacctgtgtcccattccaccaccctcctagaatagggccctgatcctcgccctgtaccccggccctggccccgcccctccaagccccttcccctgctctcctgtccccctcaccccacctggtctcctcctgcccttccactaccagggtggtggcacccgcaggtccctgcactcaaaacactcctccctccgtccctaatgcccttgcttgtttatccattttccttgtctgttgtcagcatgagtgccatgccggggggctcctcctctccaggctgcagccccccaccccgtgctgagatggatgcccggctcgaagctagtgctcagatagcccttgaatgagtgaagcagagctcctcacccaggaggctccaaaatccacccccgcaggctgccaggttgagcagggaaaactgcaggctgcctgggccgacttgaatttcagataactgtaaatcatgtttcgcataagtgttgcacgcagtactcaactggacagttgtatttatttggcggccccaccttccccccaccccaccctaccccaccctcttttttgtttaacttcatttatccacgcagttgaaagttcaaaaggtgtgaagggcgtgttgtagaaagactccttgtccccagccggcacatctcagaggggacctgtgcagtgagtttcttgtattccctccacagggattttcacggacaccgagtgaactgcgagtccccccacccttcttgctaaggtggcagtggcgggccccatggtcgcgcaccctgcatttttcctaccttggggacatccctcctgtgtgcagggtgctcgtctgtagctcctgtttttgacatggaggtggattttctgcactggcttctatgggctctgccttacgccattgctgtgtggcctggctggggcaaggtgggctcctgtggactggatggtggctgcagctgcccccgtggggctcataccaatgccatgcccattccgagtaggcatgtgtttgctcagaactgaggaatctgatttttgtttctgctttttatttgtttctcagggggctggggtgtctggattctctgctaagtgccagtgaccttgaacttgagtggctaggtgctgtgtaggcagagttgtcacaatggaggaggggacagagaaacgcatccacccccaccaggttgctcaccacagggccagcgcgctcttcctgctctgggacactcaccgaggctgaattccacgtggctcccgtggctaggcagatagatgcagctaagggcttaaaaatgtttctatcccaaaaggaggtgagaggtttccaggaaagttgcgctgtggcttatcgagtgcttgtagctgtggaggcttgtccatggggtgtttgtggggccctaggagacaggacttccggaggcagaggaatgtttcaggggctaaggagagactcagcagacctggagcccgcttaagctcatctctggtggccttgtgggtctttctcttccctgtcccttttccagtcctgtctggccaccccaactggcgaaggcctcaggcatgtgttcttggatcggggtgggggtctcaggtctgtggcatgagtgggggcgggtagggctggccgtgcattcagggcctggctgaactactgagtgtccagcatgaggctgggtctcgcaggcacgtggcaggtcatggccctgtgccatctgcggttgggggccacacaagtggaactccctggatcgctgccctgggggttggctggcctctggcttggccatttctcggggcggctagcacccctcttcctcctggctgcttctcacaggaaggagcgaatgcgtgccggccacacgtgggagggcagggtaggatgcctgtggctttcccaggcgcctcctggggaggagctgatgggtgctcaggccccagggactctctggttccaaggagcctcaagggcagtggggcctcgggcttgggacccaagccctgcctctctccctggggaggcagagcagggtatggggttgggtacagtggtgggtacttgctagcctcttctcggcctggggaaaggccagggcaccgagctgggaccccatatggcctggcctcagagtgcaccaggctttggctggctagtccccaagggcagccgctctccaaggggactgcgggttgagcaacttctttgctgtttattttttcacgggctcagggcggcgtcacacttcagggagagctggatggggatggcggccccttcctgggccgagaggggaggaggtggcacaggcccatgtccatgtctgcccgcagagccagcagccacccccgcccagccaacacctccccaggcccaagggggacgcggccctgaagatgcgggcgtcagtgcgcatgacccggtacctcgagtcctggggggcggcacggccctttgcacatctcaaccaccgtgagagcgtgagcagtggtgagacccacgtccccaacgggcggaggcctaaggtaggtccccctcccacccagaaagccagggattggggccccaagccaggagcaggagagtgagtgctgaagatctcctgccctctcagcctcactgtccccatctgtaaaatggccacagcctctgccccacctcctggggctggagaggaagcaaagacatttggagaatactccgggatggccaggaacatccctgtcctccttgatgcctggaccacgctcagctatagtcaggatgtctgtggcttggacgaccccgacaccttgtcctgtgtatcaagtgccggaggggctgaacctggcccagcagtggccttcagggcccagtgttctggggaatgactgtatcctttcctcccttccctttcagagcgttccccagcgccaccgccggaccccagacaggtgcgtgccctgccctcctggccaagtcctgctgcagccacctcctccaagcaggctgccctgagcagcctctgtccagtgggcagctccgcaaggcccagcccaggacctctgtgaagttggccagggcttgggtcaaagcatttctaggaaccagagtttcctggcccctggcccttctgcatggccacatgatggagcacctgttgtatggtgccctggcggggtcagtggagtcggtggaagggaaggagatgactgtgggtgtcctggtccccatgccaatcccgggggtgtagccgaagatgccttagactggggctcgggtctgccctctgcctctggcttggctgctggccagctgggccatgctgggccatgctgggtcctcactcgccctctctgggcctcagtgggacccacccattccttcaccatcccacctgaggttctgggaccccactggtgtctacgtccacaccctcctctgggagagttggaggtggtcgctgtgctgatgcagttgtgggtatctgattccagaagcatgtcccccaaggggcggtcggaggatgactcgtacgatgacgagatgctgtcagccattgaggggctcagctccacgaggtgaggtctgagacctctgtccacccccctctcctctccccctcggataggggtcccctatatgggcaggcccatctcataccccatgcctggtggcccagccttgttccttccccctgcctgctgccacctcctgagggaatggaccccttcccagcccccatctcaccgcagctgcccccgcccacaggccctgctgctccaagtccgatgacttctacacctttgggtccatcttcctggagaagggctttgagcgcgaggtgagggcccccagcagcctcctccgcagagggacggggtctccaggcctggggtagggtgggggacgcaggtcatggggcagcctgcgttcccaagaccggctaggggaggggcactgagaatccacagctgctcctgggcggggggcaggggcggggccagtgcagcaccagcctggctctctcctgtcggcttcattccatgacccaaatgcaccatcagagatttgcctccatgggtctgcaagacttttgctccgtccagtgccaaagccttagcagatcctggcatggatgcctcaggctgatggcaccggccttgcaatgagacgagaacccaaagctcagttatcagtgtcctgtcttgctgctatgagctttttgtactgataacgaccatttctttactgagcgccttctgggttctggcagcatcctcggcacccacattatataatttaaccgtcacaacggccagcccaggggtgctgctataccacttcacagagaggaaactggctctcagaggcttcagagcctgtccccagcttcaggtgtggctccctgagttgggggcttcctaggtgaggtcacgaggaaacctgctggccaagtgacctggcagggtgtggccagtgtggccagggccgccgagcctgctttccttccctgcagcaggaacccttctggggctgtgatcctgcgatggtgcctgggtgggagtgggggtggggggcgggatggtctccctacctgccagcttcttggtttgaggtgaggacagccccggaagctcagacttggctcctgtccatgtacttggggccatgagctctgcagggaccttggaaagagagagacgggtggtgtagggcaggggaaggcattgtcttcaaacaggaaaaagctgagaatggaaacaggcgaaacttaccaagtgtaacatcacctggaactgaaggagggtgggaaggttttaattattttaaaaatagagatggggactcactatgttgcccaggctggtctcaaactactgggctcaagtgaacctccttcctcggctcccaaagtgctgggattacaagcgtgagccactgtcccagcagggaggtgttttttttaaagctgattcactggaggcagggtgggcaagtggcactgctggtggccacccctcacagtccctgctgcccccagtaccgcctggcacccatcccccgggcccgccacgactttgcctgcgccagcctgatcttcgtctgcatcctgctcgtccatgtcctgctcatgcccaggtcagttgcagggaggggtgtgggggtccggcctgctgggatccaggctggaaggtgactatgaacctgcaaggagctgtgtgatttgggctggaaggggtcggctgctggggtcctagcaactggaccaggggctgtggcagcacaccttgagttaccaacacttctttttaatagaatgtgtgttttctgccacaggcctccctactccctaacgtctctctcctcagccacctgtcatatgtgtggcctgcatgcattttggttgacagcccttgccttaggtgtttggagtgctaggaggatagactctgaaaactgtaggcgccatcctttttctcttatatatagggaaattggggcacagaggattaatgatttatccaaaactcactgagattcatgcttctggctctagggccctgctggtggggtatagggatgagggtgaagtcagagggaagggggatctaaggtcagctacttggtgctttctagaagagcagttaggccgaagcatcgaccaggattgtggttttggctatgcttactaaagacataatagggaggctgtgcgtggtgactcacgcctgtaatcccagcactttgggaggctgcggggcgaatcactcgaggtcaggggtttgagaccagcctggccaacatggtgaaaccccgtctctaccaaaaatacaaaaattagctgggcgtggtggctggcgcctgtaatcccagctactcgggaggctgaggcaggagaatggtgtgaacctgggaggcggaggttgcagtgagccgagatcatgccactgcactccagcctgggtgacagagtgagactccatctcaaaaaaaaaaaaaaaaaaaaacgaagaaagatgtaatagggaatgcgtttttacagttttttctgagtctaaaggctgcagagacattgctcatttcttatacacttggaccaaaaagagtgatatggtcttgacctcaatcttcataatctagctgggccctgtgtattgcccatggggcagctgggccacagttttcacaggtcccctttgctgtgggcagaataccaggtagggtggggacaggtggctgtgagccagaggattggtgggggcggtggtcctgggcagaaggccctaggcagaactgaggttcttccctcctctccaggacggcggcactgggtgtgtccttcgggctggtggcctgtgtactggggctggtgctgggcctgtgctttgccaccaagttctcggtaagtggggagctctggccccgcgggccctccctccctgcctcaggacacctgcctaggagccctccctggtgagcttggctgggctgagcccctgctctggtcaaggttgggtgcccatctctcctgcctcggggacaatttcctgagtgccctggaggcttctcccgaggatacccctccccaggctgccagcgaccagccctccttgcccaggctgttggctctgggtgacttgaccctgttaccccacagaggtgctgcccagctcgggggacgctctgcactatctctgagagggtggagacacagcccctgctgaggctgaccctggccgtcctgaccatcggcagcctgctcactgtggccatcatcaacctggtgggtcccgtggtggggaggcaggcctccggggtagagggagacctccagatttgggacgcctacaatgtggccttaaaagctgcctctggttgtcctctcccccgagctcagcaggtggatgtggggggttcccctttgtacactcagggctcctcacctttgggttctcaaatgcggactttgggggcccagagagcttgtgtggccagctccaggaggatgtgatgccttgagccaccacatcctgacatcccattcattcttcacggcccttctgagactcagtttcccctctcagggatgaacccttgctcctctgctggggctcagaggctcagtgctgcagaaagcagcctgcgggacacttgtccacctctctcccagaccccgcatcctgggctagggcctctccctcacctctctgccccggaccctgggtttttggaatgtgttcctgtgaccacctccctcagggtcacctggggaggctgaaaatgttggtcccggcccctccccctggctgctgccgctctgggggtgtctgcaccctcatctcctagcccctctgccctctgaacctgacggctgctgtggggagtcggcacggcggcttgagccgtgctcagagctgatcccaacgtgaaacctcaggctgctgtgatactcatggttgcctggggcccaccttgcatggcttgggcaggttccagcgttcttgtccctggactgatggggacctgtctcctctacagcccctgatgcctttccaagttccagagctgcctgttggcaatgagacaggcctactggccgcgagcagcaagacaagagccctgtgtgagcccctcccggtgagtgcgccgggcccggctccgtggcctcattcagagtggggctgctgctgccagaggtgtagtttggaccctcaaagcatgggtgctgacccctgagagcacagcatgtggaggctgagaacagcctctcctgcagagctgggaaagcagggtcccatgggcccagctggcctctggcccaagggagtgaggagtcctggccttggcaggcttggtctaggctgtgtgggtaggcagggagtgaggggagcatctggccttgtgcgggtctctggtcagggcacttcctctctgacaggcctgtcactattttatacacgagtatgcacagctcagggaggggcttcattaggtgatggcgccagggcaggcccagacaggctgatggcagaggtcaaggtctctgcctggagggccttgcagacagcagactctgcagcttcagaggctggcagtgcagtgagctgggctctaaaaggcagaaaggttctgggacaagggacagcatgggaaaaagccgaggcgtgtgttgggagcagtggggttttgggtgggttgtctcctggagagggagtggggagaactgggagatggagccgggggcaggcttgatccagactgggaggtgcaggaatgccggggtgtgaagagcgtggtctttattctgggggctggggagccacagcacaatcttgagcagggcagaggtcctttgggagggtaagctcacaaaaactcagggaggcagcttggatgcatgcacgctccggccttgaccttgactgtggtctgttgtatccacagcacctgcatactgttttttcccgtttaaatgagctcacttcataagaaataaaactacagtgaaaacaacactggacactcttaggtctcattgttttttgttgagaagggaggtggtaaggcaagagcgtgatgctgaggtgcagggagtggggctctgtggtatgtgctgggctggaggcgagagtacgtggtggggtggccctgtcctgagtgacaccctgccccctcagtactacacctgcagctgtgtcctgggcttcatcgcctgctcggtcttcctgaggatgagcctggagccaaaggttgtgctgctgacagtggccctggtggcctacctggtgctcttcaacctctccccatgctggcagtgggactgctgcggccaaggcctgggcaacctcaccaagcccaacggcaccaccaggtggggtcccgcccgtccccgtccccatccccatggtggcctgttcatctggtgcctgcctgctcgcaccaaggggcttctgtgttcatggggagtgggcaccttgcagggcggggctggcagatggtgtccagcgctcagagccgaggaattcactggcaggttctttaggaagtccctagtctgacagaggccaccgggtcatcctgtttccaggccagtctagtggtggggagggaaggaggctgcagctctagctctgtgcctctcaaggccacatcctgggtctcgagttctggaaggagcagttgcttccatctcttcctggacaggtggtgatacctgaggctggcagggctggggtgtgtggttctaggggttctgggtcccaagtggattaaggagcggacctggcccccctaaacaaaaaagctggaagcctgggacccagaacagaacctgctcctgtgggtctgccccgcacctgcaaaatccttatgccacccatccaccccaccccgcctgccactgaccctggctcaccagttctgggctcccttcaggccctcatcacagctgattctggtcattcaggggtgagatcagaggtcttccccacaagggctcccccatctccctgcccacctctcccctcccccctttataataccccaaagttgtcttgttttctacccaccccattagaatcaattttcccctttcccctgggtgtgcgtggcacctggagagcatgactcagtgggttggagtggtgagtgctgggagtgacttgggcctcccttcgcattcagtggcacccctagctgttcctggaaggacctgaagaccatgaccaatttctacctggtcctgttctacatcaccctgcttacactctccagacaggtaaggaggctggccccccccccccccccaagctctgcccacttttcctcacctccatctggagatggggtggggtggggtgggctcaggtggagtagcagaaaagactagagtcctgccaggaaagcactggtcccctggccagaggctccaaggacgccagggacagacagacctggctaggagatgcacagcagcacggctcccacttgcctgtggacggggcgcttatgttgctgccgtttgcagattgactattactgccgcttggactgcctatggaagaagaagttcaagaaggagcacgaggagtttgagaccatggagaacgtgaaccgccttcttctggagaacgtcctgccagcccacgtggctgcccactttatcggtgacaagttaaacgaggtgtgctgagaaggggctggggcgggggcagggaggcggacggtccaggcgcagtccgtagggtgaaggtgtggagctggagtgcatgctgagcttggcttcctcaggtcctggcctcaccgggatgcccaggcgacaatgtatggcatcagctgtgagagcatgggcctggggtcagggacctgggctcaagtgtggtcgtgaacaaggcaccttgctttcccaggccttggtcttcacctctgtaaaatgggctgacagcccttccccacaggcttgtggcgaggatgaaatgtgtgttcaggggtttgtgatctggacattctgtgttgatgaaaatgtctgtacttctactagttatgtgtagcaagttttctagataggaaacggaggcccagtgagaatgaagacctcccgttaccctccactactgcagaggttctcaccccatgtgtacatggaaacacccatggtgcttctgtgagcaaaaaccaacctgagcctgaccctccctggccacccaattcttaatcacccggtaacagccaccagatgaaactcatgcagcccgcccccctccccactccccgaaagctgggctgggcagttcagcagtgccatctgctcctgaggccttgccactcttcctgtcccctcaagaggtgaagtggtgtaaggtccggtttcttcccatccaggactggtaccatcagtcctatgactgcgtctgtgtcatgtttgcctccgtgccggacttcaaagtgttctacacagagtgcgatgtcaacaaagaagggctggagtgcctacgcctgctcaatgagatcattgccgacttcgacgaggtacagcctctagcccagccttgcgcagcagcccccacccatgctggagagggaagggcggtggcacctgccatcctaaaacccaatttaaaaatgtgacacagagctgggcaaggtggtctataatcccagcactttgggaggtcaaggtgggtggatcacttgaggctaggagttcgagaccagcctggccaacatggcgaaaccctgtctctactgaaaatacaaaaaattacaagctgggcttggggcgcacacctgtaatcccagctacttgggaggctgaggcaggagaatcgcttgaacctgggaggcggttgcagtgagctgggatcacgccactgcactacagcctgggcaacagtacacaactctgtctcaaacaacaacaacaacaacaaaaaaaaaacgacgtggcccgcctggggggaacatacattacacagaatataaaggtacacatttctttttcattctgttttatgcagcaaataattcgttggcatcttctctgtgatgggcagcttgctaagattagactcaggccccttagcttcatttccaactaagcccacgctatcaaccaagccaaacagaggaaatcagtttgggttgaattctttgctggagacaaagaatctacattcctgtgtagataatgctgtgtttgctctgtgcagacacagatggaagggaagagggagacagtgtggggacggagacaacaggaagagcaggagccgccaagggccatgtcctcaccatgcttagtcatgcgctcagctggcagggcagccatgagacgtgtgtgcgagaggcggcgatgggtggagggaaccccacacccttcctgagaagcaggggcagcctggctgcggctatggagtggcggctgcttcaccgcttccttcttgcctgcagctcctactgaagcccaagttcagcggcgtggagaagatcaagaccatcggcagcacgtacatggcagctgcagggctcagcgtcgcctcagggcacgagaaccaggtactcaagcccaagaggtgaaattcagctgactgtcctcacttaaaggtgacctgtcaccttacttaaagggtgtgcccttatctcgtcctgggggaggggcacaggtacttgtagggctcagccaggattttagtggtggggatcctcaacaagagtggcgcgccagggcccactaggtctgggggctctggagatgcaaggatctgacccacagcctgtcccgcaggagctggagcggcagcatgcccacattggtgtcatggtggagttcagcatcgccctgatgagtaagctggacggcatcaacaggcactccttcaactccttccgcctccgcgtcggtgagcccgggtgatggagcggggtggggagcccctgcctctaggccagtccacccagtctgtgtggcacagctgcacctcgccatctattatgaaaggttttagaaatccctctgatctgacaagctcagcagttggacaattattctgatgttttgcatatagttagcatatagttaccattgagagttttaataatacgtaactacagaaaatggagaaccaaaattattgtctgtaaacaggtgaccttaaaatatagagaaggaagcaaagttactaaacctaaatagttgcgggcctgcgctcaaagccaggcctttgttaatacttttaacaaagtattctttgttaaaagagagattagcagatacaagtgttaacatcaaacccagacttctgagtctgaaaaagagctggccggggaatgccctcctcataccccaagcccgactgcaacgcagtgactctgggaagcaacccagccttcactctagttgttttgtccccgcatcctgtgctggggtatggattctctggcctcctctaagggcagaagcttgaggctttgcctgcacgcttgggtaactgtaaacatcatcttcaggcataaaccatgggcctgtgattgctggagtgattggggcccgaaaacctcagtatgacatctggggaaacactgtcaatgtggccagccgaatggaaagcactggagaacttgggaaaatccaggtaaagacctattggggaagcagttgactaaggggaaaagatcttccccagaggtgaggggacttggacttaagagctgctgtctcacccagcagccatggttctagctatcacactctccagggaaggcagactgcatgcctgggcagtctctatctgtccctaccatgacaggtgttcttcccgcctctgaagacaacaggtctctcttccttttcaggttaccgaggagacctgcaccatcctccagggcctcgggtactcttgtgaatgccgtggcctgatcaacgtcaaaggcaaaggcgagctgaggacttactttgtctgtacggacactgccaagtttcaggggctggggctgaactgagggctcctgctggattccgaaaaggccgggaagccagtctccttccctgaagcaagcccaggagaagactctccgccccacgccaatcccaaaggcatgcagatggctgtgcatgttggcttctttggacctgcactggaggatttctcagacacatgcaccagattctggctcgaagcagccactgagccataatgcgcaggggaggccagaagctctgtgcctggtctgtaacagtttccaggccagctggagaatgttcactggttcggggctgactttgagatctttgttccctgaggtgccaggcaggcaactttagcacatgatgaaaacagacttccacctcagtggcctgtgggcacgcacaagtgaggtctgtttttctagacaccaagggggagtaagctgagctgtctagcacggattggagactccctctccctggtgggcctggcaatgacagcatttctcacagaggcattctggtaaatgaagctgaaaggggtgttttacatctgtaaacggtttcaaacaggtagagagaaaaacaccacaattaacactgttactttttgccttgtctggcatgtttgttttaaatgaatacattaatggggtttttatccttttgaatgacttttcagacactagacataaatctcttccctccagtgtatgctctgcctttttaaccactgacatgtaaggaggactactgtctagcatcagcttatggggtcagctggctgtggggatagagtcctgaggaatgtggtcacagcaagaaggcggggagcagcagagccttgcctttgaatgaggcagcttgtgaggcaagcattctggagagaggtgctttgaaagtaaggtgcggcctttcacctcttccttgattactcacacatctttgcgttctcccctgccgtccttcaactgtatcttacttttcttaccagaaaggaatggagtctgtttagagacaacttggacaacctgtgagtgcatctcttctttcctttagtcttcacagctaactctggagagcttcaaaactagaaggatctactccgcatgggtgcatgcagaggctcctggatctgggaagcccgccccctcacaaatgctgagccgttcttgctctgaaactgcgtgagtcaaggcaaatgcaaaaagccaggttttggggatgtgtcttactgtgcttcaacttcccaaggaattgaaagtcaacctaactgtaacaacagggtgagaaatgaccaaactgcccgtgactttttctgaatggacttcataaccggaagacttaaccggtggcctcatcaccagagcatcgccaggatttctaatgcactcagtttccctacatagcagggattcttagctaggtgtccccatgaaccccgtaaagttctacacaaagtcttgcatacaggagcctttacaagatgattatacagggttgcagattgggtgactgaccagacttgttggggtcctgggatgagttgccccgggctgcaaattaagagtacagctaagtgcgggggtggcggtggagggaacgaaaattgaacctgtctgcctgtgctgtgtcgtgtggctttatcagcccgaggaagggcaggtgtattctaatttgcacaaaggtgctgggtagactagtggcagctctcatgtgctgcacataagtggaatcagtatgaatagaagaacttgctgtataaaggaatttcatggcaacaatgctggtaagggcaattagcctcgcttaagttgccttttttacacaccaaaactttttacatgaagggctggtttcacatgaatactatactgaaatctgtgctctcaagatctagcagtgaccagggctgcccggcgggggctctcctggcaagtcaggaaggtttctgttgctaatataacatagaaacacattagtgcactgggcctctctgaggtcagcatatttgtactcttggaatatttgtttttttcttcagtaacaacagaaaccccagttgggagtttaacaaataactgactaccactcactcatgcatttttatttccaattaaagcaaagcactgtgctgtgctcagataataatagtttgtaagtaaaagtttttagttttcagtgttcaggttatagaatataactgaccataaaaattacctgcaggtattttctttttatgaacttgtttttaaattaccaagtaattactggtgtcattttgttttatgacagacacacgtatctaacaaacaaacaaacagtgaccttctccatgggtcaaggacttccttacaatttctcctgagttaacttttgtgaaaataatacctaaggttttctggcttattgaggaaatttcctaacaaacaaacaaacaaacaaacagaagagaagatcattaaccactgtatactttgtgtatataataggtcagtgtaaagaaatatgatttgaggtggtgcatgcaagtaactagggtttattctatataatgaatatttatagatctgtaacatttgtttcaaaatgctgtttcatttttataaagtaccagtgtttagctgctttttatacattaaattagcaatttgaaaaactcaaa

SEQ ID NO: 3 - DNA, 2496 bp, homo sapien
acucucauuagguagcguggaaggagccuucggauggagcugaggaacugcguguggagucagcccagucuggaugcacaggaggaugcuggcggcacagugagugaggccuggugccagagcugugcggaccccuuguuggccauggagcagcaggcccagaggcccucuccccagcccugcuugccugccucggagaggacagaggccuaggcccuggggccuccuuaacggccccuuaacgacacgcgugccaaggguggaggaugccagccaaggggcgcuacuuccucaacgagggcgaggagggcccugaccaagaugcgcucuacgagaaguaccagcucaccagccagcaugggccgcugcugcucacgcuccugcugguggccgccacugccugcguggcccucaucaucauugccuucagccagggggaccccuccagacaccaggccauucugggcauggcguuccuggugcuggcgguguuugcggcccucucugugcugauguacgucgagugucuccugcggcgcuggcucagggccuuggcgcugcucaccugggccugcuugguggcgcugggcuaugugcugguguucgacgcauggacaaaggcggccugugcgugggagcaggugcccuucuuccuguucauugucuucgugguguacacacuacugcccuucagcaugcggggcgcugucgccguuggggccgucuccacugccucccaccuccuggugcucgguucuuugaugggaggcuucacgacacccaguguccggguggggcugcagcugcuggccaacgcagucaucuuccugugugggaaccugacaggcgccuuccacaagcaccaaaugcaggaugcgucccgggaccucuucaccuacacugugaagugcauccagauccgccggaagcugcgcaucgagaagcgccagcaggagaaccugcugcugucagugcuuccggcccacaucuccaugggcaugaagcuggccaucaucgaacggcucaaggagcauggugaccgucgcugcaugccugacaacaacuuccacagccucuacgucaagaggcaccagaaugucagcauccucuaugcggacaucgugggcuucacgcagcuggccagcgacuguucucccaaggagcuggugguggugcugaaugagcucuuuggcaaguucgaccagaucgccaaggccaacgagugcaugcgaaucaagauccucggcgacugcuacuacuguguaucgggccugcccgugucgcugccuacccacgcccggaacugcgugaagauggggcuggacaugugccaggccaucaagcaggugcgggaggccacgggcguggacaucaacaugcgugugggcauacacucggggaaugugcugugcggggucaucgggcugcgcaaguggcaguaugacguguggucccacgacgugucccuggccaaccggauggaggcagccggaguacccggccgggugcacaucacggaggccacgcuaaagcaccuggacaaggcguacgagguggaggaugggcgcgggcagcagcgggaccccuaccucaaggagaugaacauccgcaccuaccuggucaucgacccccggagccagcagccacccccgcccagccaacaccuccccaggcccaagggggacgcggcccugaagaugcgggcgucagugcgcaugacccgguaccucgaguccuggggggcggcacggcccuuugcacaucucaaccaccgugagagcgugagcaguggugagacccacguccccaacgggcggaggccuaagagcguuccccagcgccaccgccggaccccagacagaagcaugucccccaaggggcggucggaggaugacucguacgaugacgagaugcugucagccauugaggggcucagcuccacgaggcccugcugcuccaaguccgaugacuucuacaccuuuggguccaucuuccuggagaagggcuuugagcgcgaguaccgccuggcacccaucccccgggcccgccacgacuuugccugcgccagccugaucuucgucugcauccugcucguccauguccugcucaugcccaggacggcggcacuggguguguccuucgggcugguggccuguguacuggggcuggugcugggccugugcuuugccaccaaguucucgaggugcugcccagcucgggggacgcucugcacuaucucugagaggguggagacacagccccugcugaggcugacccuggccguccugaccaucggcagccugcucacuguggccaucaucaaccugccccugaugccuuuccaaguuccagagcugccuguuggcaaugagacaggccuacuggccgcgagcagcaagacaagagcccugugugagccccucccgcaccugcauacuguuuuuucccguuuaaaugagcucacuucauaagaaauaaaacuacagugaaaacaac

SEQ ID NO: 4 - DNA, 6196 bp, homo sapien
ugaggaacugcguguggagucagcccagucuggaugcacaggaggaugcuggcggcacagugagugaggccuggugccagagcugugcggaccccuuguuggccauggagcagcaggcccagaggcccucuccccagcccugcuugccugccucggagaggacagaggccuaggcccacgggggaggguguuggcagacagaugcccuccaggcccuggggccuccuuaacggccccuuaacgacacgcgugccaaggguggaggaugccagccaaggggcgcuacuuccucaacgagggcgaggagggcccugaccaagaugcgcucuacgagaaguaccagcucaccagccagcaugggccgcugcugcucacgcuccugcugguggccgccacugccugcguggcccucaucaucauugccuucagccagggggaccccuccagacaccaggccauucugggcauggcguuccuggugcuggcgguguuugcggcccucucugugcugauguacgucgagugucuccugcggcgcuggcucagggccuuggcgcugcucaccugggccugcuugguggcgcugggcuaugugcugguguucgacgcauggacaaaggcggccugugcgugggagcaggugcccuucuuccuguucauugucuucgugguguacacacuacugcccuucagcaugcggggcgcugucgccguuggggccgucuccacugccucccaccuccuggugcucgguucuuugaugggaggcuucacgacacccaguguccggguggggcugcagcugcuggccaacgcagucaucuuccugugugggaaccugacaggcgccuuccacaagcaccaaaugcaggaugcgucccgggaccucuucaccuacacugugaagugcauccagauccgccggaagcugcgcaucgagaagcgccagcaggagaaccugcugcugucagugcuuccggcccacaucuccaugggcaugaagcuggccaucaucgaacggcucaaggagcauggugaccgucgcugcaugccugacaacaacuuccacagccucuacgucaagaggcaccagaaugucagcauccucuaugcggacaucgugggcuucacgcagcuggccagcgacuguucucccaaggagcuggugguggugcugaaugagcucuuuggcaaguucgaccagaucgccaaggccaacgagugcaugcgaaucaagauccucggcgacugcuacuacuguguaucgggccugcccgugucgcugccuacccacgcccggaacugcgugaagauggggcuggacaugugccaggccaucaagcaggugcgggaggccacgggcguggacaucaacaugcgugugggcauacacucggggaaugugcugugcggggucaucgggcugcgcaaguggcaguaugacguguggucccacgacgugucccuggccaaccggauggaggcagccggaguacccggccgggugcacaucacggaggccacgcuaaagcaccuggacaaggcguacgagguggaggaugggcacgggcagcagcgggaccccuaccucaaggagaugaacauccgcaccuaccuggucaucgacccccggagccagcagccacccccgcccagccaacaccuccccaggcccaagggggacgcggcccugaagaugcgggcgucagugcgcaugacccgguaccucgaguccuggggggcggcacggcccuuugcacaucucaaccaccgugagagcgugagcaguggugagacccacguccccaacgggcggaggccuaagagcguuccccagcgccaccgccggaccccagacagaagcaugucccccaaggggcggucggaggaugacucguacgaugacgagaugcugucagccauugaggggcucagcuccacgaggcccugcugcuccaaguccgaugacuucuacaccuuuggguccaucuuccuggagaagggcuuugagcgcgaguaccgccuggcacccaucccccgggcccgccacgacuuugccugcgccagccugaucuucgucugcauccugcucguccauguccugcucaugcccaggacggcggcacuggguguguccuucgggcugguggccuguguacuggggcuggugcugggccugugcuuugccaccaaguucucgaggugcugcccagcucgggggacgcucugcacuaucucugagaggguggagacacagccccugcugaggcugacccuggccguccugaccaucggcagccugcucacuguggccaucaucaaccugccccugaugccuuuccaaguuccagagcugccuguuggcaaugagacaggccuacuggccgcgagcagcaagacaagagcccugugugagccccucccguacuacaccugcagcuguguccugggcuucaucgccugcucggucuuccugaggaugagccuggagccaaagguugugcugcugacaguggcccugguggccuaccuggugcucuucaaccucuccccaugcuggcagugggacugcugcggccaaggccugggcaaccucaccaagcccaacggcaccaccaguggcaccccuagcuguuccuggaaggaccugaagaccaugaccaauuucuaccugguccuguucuacaucacccugcuuacacucuccagacagauugacuauuacugccgcuuggacugccuauggaagaagaaguucaagaaggagcacgaggaguuugagaccauggagaacgugaaccgccuucuucuggagaacguccugccagcccacguggcugcccacuuuaucggugacaaguuaaacgaggacugguaccaucaguccuaugacugcgucugugucauguuugccuccgugccggacuucaaaguguucuacacagagugcgaugucaacaaagaagggcuggagugccuacgccugcucaaugagaucauugccgacuucgacgagcuccuacugaagcccaaguucagcggcguggagaagaucaagaccaucggcagcacguacauggcagcugcagggcucagcgucgccucagggcacgagaaccaggagcuggagcggcagcaugcccacauuggugucaugguggaguucagcaucgcccugaugaguaagcuggacggcaucaacaggcacuccuucaacuccuuccgccuccgcgucggcauaaaccaugggccugugauugcuggagugauuggggcccgaaaaccucaguaugacaucuggggaaacacugucaauguggccagccgaauggaaagcacuggagaacuugggaaaauccagguuaccgaggagaccugcaccauccuccagggccucggguacucuugugaaugccguggccugaucaacgucaaaggcaaaggcgagcugaggacuuacuuugucuguacggacacugccaaguuucaggggcuggggcugaacugagggcuccugcuggauuccgaaaaggccgggaagccagucuccuucccugaagcaagcccaggagaagacucuccgccccacgccaaucccaaaggcaugcagauggcugugcauguuggcuucuuuggaccugcacuggaggauuucucagacacaugcaccagauucuggcucgaagcagccacugagccauaaugcgcaggggaggccagaagcucugugccuggucuguaacaguuuccaggccagcuggagaauguucacugguucggggcugacuuugagaucuuuguucccugaggugccaggcaggcaacuuuagcacaugaugaaaacagacuuccaccucaguggccugugggcacgcacaagugaggucuguuuuucuagacaccaagggggaguaagcugagcugucuagcacggauuggagacucccucucccuggugggccuggcaaugacagcauuucucacagaggcauucugguaaaugaagcugaaagggguguuuuacaucuguaaacgguuucaaacagguagagagaaaaacaccacaauuaacacuguuacuuuuugccuugucuggcauguuuguuuuaaaugaauacauuaaugggguuuuuauccuuuugaaugacuuuucagacacuagacauaaaucucuucccuccaguguaugcucugccuuuuuaaccacugacauguaaggaggacuacugucuagcaucagcuuauggggucagcuggcuguggggauagaguccugaggaauguggucacagcaagaaggcggggagcagcagagccuugccuuugaaugaggcagcuugugaggcaagcauucuggagagaggugcuuugaaaguaaggugcggccuuucaccucuuccuugauuacucacacaucuuugcguucuccccugccguccuucaacuguaucuuacuuuucuuaccagaaaggaauggagucuguuuagagacaacuuggacaaccugugagugcaucucuucuuuccuuuagucuucacagcuaacucuggagagcuucaaaacuagaaggaucuacuccgcaugggugcaugcagaggcuccuggaucugggaagcccgcccccucacaaaugcugagccguucuugcucugaaacugcgugagucaaggcaaaugcaaaaagccagguuuuggggaugugucuuacugugcuucaacuucccaaggaauugaaagucaaccuaacuguaacaacagggugagaaaugaccaaacugcccgugacuuuuucugaauggacuucauaaccggaagacuuaaccgguggccucaucaccagagcaucgccaggauuucuaaugcacucaguuucccuacauagcagggauucuuagcuagguguccccaugaaccccguaaaguucuacacaaagucuugcauacaggagccuuuacaagaugauuauacaggguugcagauugggugacugaccagacuuguugggguccugggaugaguugccccgggcugcaaauuaagaguacagcuaagugcggggguggcgguggagggaacgaaaauugaaccugucugccugugcugugucguguggcuuuaucagcccgaggaagggcagguguauucuaauuugcacaaaggugcuggguagacuaguggcagcucucaugugcugcacauaaguggaaucaguaugaauagaagaacuugcuguauaaaggaauuucauggcaacaaugcugguaagggcaauuagccucgcuuaaguugccuuuuuuacacaccaaaacuuuuuacaugaagggcugguuucacaugaauacuauacugaaaucugugccacaccaaaacuuuuuacaugaagggcugguuucacaugaauacuauacugaaaucugugcucucaagaucuagcagugaccagggcugcccggcgggggcucuccuggcaagucaggaagguuucuguugcuaauauaacauagaaacacauuagugcacugggccucucugaggucagcauauuuguacucuuggaauauuuguuuuuuucuucaguaacaacagaaaccccaguugggaguuuaacaaauaacugacuaccacucacucaugcauuuuuauuuccaauuaaagcaaagcacugugcugugcucagauaauaauaguuuguaaguaaaaguuuuuaguuuucaguguucagguuauagaauauaacugaccauaaaaauuaccugcagguauuuucuuuuuaugaacuuguuuuuaaauuaccaaguaauuacuggugucauuuuguuuuaugacagacacacguaucuaacaaacaaacaaacagugaccuucuccaugggucaaggacuuccuuacaauuucuccugaguuaacuuuugugaaaauaauaccuaagguuuucuggcuuauugaggaaauuuccuaacaaacaaacaaacaaacaaacagaagagaagaucauuaaccacuguauacuuuguguauauaauaggucaguguaaagaaauaugauuugagguggugcaugcaaguaacuaggguuuauucuauauaaugaauauuuauagaucuguaacauuuguuucaaaaugcuguuucauuuuuauaaaguaccaguguuuagcugcuuuuuauacauuaaauuagcaauuugaaaaacuc

SEQ ID NO: 5 - DNA, 4735 bp, homo sapien
ggguguuggcagacagaugcccuccaggcccuggggccuccuuaacggccccuuaacgacacgcgugccaaggguggaggaugccagccaaggggcgcuacuuccucaacgagggcgaggagggcccugaccaagaugcgcucuacgagaaguaccagcucaccagccagcaugggccgcugcugcucacgcuccugcugguggccgccacugccugcguggcccucaucaucauugccuucagccagggggaccccuccagacaccaggccauucugggcauggcguuccuggugcuggcgguguuugcggcccucucugugcugauguacgucgagugucuccugcggcgcuggcucagggccuuggcgcugcucaccugggccugcuugguggcgcugggcuaugugcugguguucgacgcauggacaaaggcggccugugcgugggagcaggugcccuucuuccuguucauugucuucgugguguacacacuacugcccuucagcaugcggggcgcugucgccguuggggccgucuccacugccucccaccuccuggugcucgguucuuugaugggaggcuucacgacacccaguguccggguggggcugcagcugcuggccaacgcagucaucuuccugugugggaaccugacaggcgccuuccacaagcaccaaaugcaggaugcgucccgggaccucuucaccuacacugugaagugcauccagauccgccggaagcugcgcaucgagaagcgccagcaggagaaccugcugcugucagugcuuccggcccacaucuccaugggcaugaagcuggccaucaucgaacggcucaaggagcauggugaccgucgcugcaugccugacaacaacuuccacagccucuacgucaagaggcaccagaaugucagcauccucuaugcggacaucgugggcuucacgcagcuggccagcgacuguucucccaaggagcuggugguggugcugaaugagcucuuuggcaaguucgaccagaucgccaaggccaacgagugcaugcgaaucaagauccucggcgacugcuacuacuguguaucgggccugcccgugucgcugccuacccacgcccggaacugcgugaagauggggcuggacaugugccaggccaucaagcaggugcgggaggccacgggcguggacaucaacaugcgugugggcauacacucggggaaugugcugugcggggucaucgggcugcgcaaguggcaguaugacguguggucccacgacgugucccuggccaaccggauggaggcagccggaguacccggccgggugcacaucacggaggccacgcuaaagcaccuggacaaggcguacgagguggaggaugggcacgggcagcagcgggaccccuaccucaaggagaugaacauccgcaccuaccuggucaucgacccccggagccagcagccacccccgcccagccaacaccuccccaggcccaagggggacgcggcccugaagaugcgggcgucagugcgcaugacccgguaccucgaguccuggggggcggcacggcccuuugcacaucucaaccaccgugagagcgugagcaguggugagacccacguccccaacgggcggaggccuaagagcguuccccagcgccaccgccggaccccagacagaagcaugucccccaaggggcggucggaggaugacucguacgaugacgagaugcugucagccauugaggggcucagcuccacgaggcccugcugcuccaaguccgaugacuucuacaccuuuggguccaucuuccuggagaagggcuuugagcgcgaguaccgccuggcacccaucccccgggcccgccacgacuuugccugcgccagccugaucuucgucugcauccugcucguccauguccugcucaugcccaggacggcggcacuggguguguccuucgggcugguggccuguguacuggggcuggugcugggccugugcuuugccaccaaguucucgaggugcugcccagcucgggggacgcucugcacuaucucugagaggguggagacacagccccugcugaggcugacccuggccguccugaccaucggcagccugcucacuguggccaucaucaaccugccccugaugccuuuccaaguuccagagcugccuguuggcaaugagacaggccuacuggccgcgagcagcaagacaagagcccugugugagccccucccguacuacaccugcagcuguguccugggcuucaucgccugcucggucuuccugaggaugagccuggagccaaagguugugcugcugacaguggcccugguggccuaccuggugcucuucaaccucuccccaugcuggcagugggacugcugcggccaaggccugggcaaccucaccaagcccaacggcaccaccaguggcaccccuagcuguuccuggaaggaccugaagaccaugaccaauuucuaccugguccuguucuacaucacccugcuuacacucuccagacagauugacuauuacugccgcuuggacugccuauggaagaagaaguucaagaaggagcacgaggaguuugagaccauggagaacgugaaccgccuucuucuggagaacguccugccagcccacguggcugcccacuuuaucggugacaaguuaaacgaggacugguaccaucaguccuaugacugcgucugugucauguuugccuccgugccggacuucaaaguguucuacacagagugcgaugucaacaaagaagggcuggagugccuacgccugcucaaugagaucauugccgacuucgacgagcuccuacugaagcccaaguucagcggcguggagaagaucaagaccaucggcagcacguacauggcagcugcagggcucagcgucgccucagggcacgagaaccaggagcuggagcggcagcaugcccacauuggugucaugguggaguucagcaucgcccugaugaguaagcuggacggcaucaacaggcacuccuucaacuccuuccgccuccgcgucggcauaaaccaugggccugugauugcuggagugauuggggcccgaaaaccucaguaugacaucuggggaaacacugucaauguggccagccgaauggaaagcacuggagaacuugggaaaauccagguuaccgaggagaccugcaccauccuccagggccucggguacucuugugaaugccguggccugaucaacgucaaaggcaaaggcgagcugaggacuuacuuugucuguacggacacugccaaguuucaggggcuggggcugaacugagggcuccugcuggauuccgaaaaggccgggaagccagucuccuucccugaagcaagcccaggagaagacucuccgccccacgccaaucccaaaggcaugcagauggcugugcauguuggcuucuuuggaccugcacuggaggauuucucagacacaugcaccagauucuggcucgaagcagccacugagccauaaugcgcaggggaggccagaagcucugugccuggucuguaacaguuuccaggccagcuggagaauguucacugguucggggcugacuuugagaucuuuguucccugaggugccaggcaggcaacuuuagcacaugaugaaaacagacuuccaccucaguggccugugggcacgcacaagugaggucuguuuuucuagacaccaagggggaguaagcugagcugucuagcacggauuggagacucccucucccuggugggccuggcaaugacagcauuucucacagaggcauucugguaaaugaagcugaaagggguguuuuacaucuguaaacgguuucaaacagguagagagaaaaacaccacaauuaacacuguuacuuuuugccuugucuggcauguuuguuuuaaaugaauacauuaaugggguuuuuauccuuuugaaugacuuuucagacacuagacauaaaucucuucccuccaguguaugcucugccuuuuuaaccacugacauguaaggaggacuacugucuagcaucagcuuauggggucagcuggcuguggggauagaguccugaggaauguggucacagcaagaaggcggggagcagcagagccuugccuuugaaugaggcagcuugugaggcaagcauucuggagagaggugcuuugaaaguaaggugcggccuucaccucuuccuugauuacucacacaucuuugcguucuccccugccguccuucaacuguaucuuacuuuucuuaccagaaaggaauggagucuguuuagagacaacuuggacaaccugugagugcaucucuucuuuccuuuagucuucacagcuaacucuggagagcuucaaaacuagaaggaucuacuccgcaugggugcaugcagaggcuccuggaucugggaagcccgcccccucacaaaugcugagccguucuugcucugaaacugcgugagucaaggcaaaugcaaaaagccagguuuuggggaugugucuuacugugcuucaacuucccaaggaauugaaagucaaccuaacuguaacaacagggugagaaaugaccaaacugcccgugacuuuuucugaauggacuucauaaccggaagacuuaaccgguggccucaucaccagagcaucgccaggauuucuaaugcacucaguuucccuacauagcagggauucuuagcuagguguccccaugaaccccguaaa

SEQ ID NO: 6 - DNA, 3281 bp, homo sapien
gcgaucgcuuucgaaggagauagaaccaugccagccaaggggcgcuacuuccucaacgagggcgaggagggcccugaccaagaugcgcucuacgagaaguaccagcucaccagccagcaugggccgcugcugcucacgcuccugcugguggccgccacugccugcguggcccucaucaucauugccuucagccagggggaccccuccagacaccaggccauucugggcauggcguuccuggugcuggcgguguuugcggcccucucugugcugauguacgucgagugucuccugcggcgcuggcucagggccuuggcgcugcucaccugggccugcuugguggcgcugggcuaugugcugguguucgacgcauggacaaaggcggccugugcgugggagcaggugcccuucuuccuguucauugucuucgugguguacacacuacugcccuucagcaugcggggcgcugucgccguuggggccgucuccacugccucccaccuccuggugcucgguucuuugaugggaggcuucacgacacccaguguccggguggggcugcagcugcuggccaacgcagucaucuuccugugugggaaccugacaggcgccuuccacaagcaccaaaugcaggaugcgucccgggaccucuucaccuacacugugaagugcauccagauccgccggaagcugcgcaucgagaagcgccagcaggagaaccugcugcugucagugcuuccggcccacaucuccaugggcaugaagcuggccaucaucgaacggcucaaggagcauggugaccgucgcugcaugccugacaacaacuuccacagccucuacgucaagaggcaccagaaugucagcauccucuaugcggacaucgugggcuucacgcagcuggccagcgacuguucucccaaggagcuggugguggugcugaaugagcucuuuggcaaguucgaccagaucgccaaggccaacgagugcaugcgaaucaagauccucggcgacugcuacuacuguguaucgggccugcccgugucgcugccuacccacgcccggaacugcgugaagauggggcuggacaugugccaggccaucaagcaggugcgggaggccacgggcguggacaucaacaugcgugugggcauacacucggggaaugugcugugcggggucaucgggcugcgcaaguggcaguaugacguguggucccacgacgugucccuggccaaccggauggaggcagccggaguacccggccgggugcacaucacggaggccacgcuaaagcaccuggacaaggcguacgagguggaggaugggcacgggcagcagcgggaccccuaccucaaggagaugaacauccgcaccuaccuggucaucgacccccggagccagcagccacccccgcccagccaacaccuccccaggcccaagggggacgcggcccugaagaugcgggcgucagugcgcaugacccgguaccucgaguccuggggggcggcacggcccuuugcacaucucaaccaccgugagagcgugagcaguggugagacccacguccccaacgggcggaggccuaagagcguuccccagcgccaccgccggaccccagacagaagcaugucccccaaggggcggucggaggaugacucguacgaugacgagaugcugucagccauugaggggcucagcuccacgaggcccugcugcuccaaguccgaugacuucuacaccuuuggguccaucuuccuggagaagggcuuugagcgcgaguaccgccuggcacccaucccccgggcccgccacgacuuugccugcgccagccugaucuucgucugcauccugcucguccauguccugcucaugcccaggacggcggcacuggguguguccuucgggcugguggccuguguacuggggcuggugcugggccugugcuuugccaccaaguucucgaggugcugcccagcucgggggacgcucugcacuaucucugagaggguggagacacagccccugcugaggcugacccuggccguccugaccaucggcagccugcucacuguggccaucaucaaccugccccugaugccuuuccaaguuccagagcugccuguuggcaaugagacaggccuacuggccgcgagcagcaagacaagagcccugugugagccccucccguacuacaccugcagcuguguccugggcuucaucgccugcucggucuuccugaggaugagccuggagccaaagguugugcugcugacaguggcccugguggccuaccuggugcucuucaaccucuccccaugcuggcagugggacugcugcggccaaggccugggcaaccucaccaagcccaacggcaccaccaguggcaccccuagcuguuccuggaaggaccugaagaccaugaccaauuucuaccugguccuguucuacaucacccugcuuacacucuccagacagauugacuauuacugccgcuuggacugccuauggaagaagaaguucaagaaggagcacgaggaguuugagaccauggagaacgugaaccgccuucuucuggagaacguccugccagcccacguggcugcccacuuuaucggugacaaguuaaacgaggacugguaccaucaguccuaugacugcgucugugucauguuugccuccgugccggacuucaaaguguucuacacagagugcgaugucaacaaagaagggcuggagugccuacgccugcucaaugagaucauugccgacuucgacgagcuccuacugaagcccaaguucagcggcguggagaagaucaagaccaucggcagcacguacauggcagcugcagggcucagcgucgccucagggcacgagaaccaggagcuggagcggcagcaugcccacauuggugucaugguggaguucagcaucgcccugaugaguaagcuggacggcaucaacaggcacuccuucaacuccuuccgccuccgcgucggcauaaaccaugggccugugauugcuggagugauuggggcccgaaaaccucaguaugacaucuggggaaacacugucaauguggccagccgaauggaaagcacuggagaacuugggaaaauccagguuaccgaggagaccugcaccauccuccagggccucggguacucuugugaaugccguggccugaucaacgucaaaggcaaaggcgagcugaggacuuacuuugucuguacggacacugccaaguuucaggggcuggggcugaacuacguaguuuaaac

SEQ ID NO: 7 - DNA, 2496 bp, homo sapien
acucucauuagguagcguggaaggagccuucggauggagcugaggaacugcguguggagucagcccagucuggaugcacaggaggaugcuggcggcacagugagugaggccuggugccagagcugugcggaccccuuguuggccauggagcagcaggcccagaggcccucuccccagcccugcuugccugccucggagaggacagaggccuaggcccuggggccuccuuaacggccccuuaacgacacgcgugccaaggguggaggaugccagccaaggggcgcuacuuccucaacgagggcgaggagggcccugaccaagaugcgcucuacgagaaguaccagcucaccagccagcaugggccgcugcugcucacgcuccugcugguggccgccacugccugcguggcccucaucaucauugccuucagccagggggaccccuccagacaccaggccauucugggcauggcguuccuggugcuggcgguguuugcggcccucucugugcugauguacgucgagugucuccugcggcgcuggcucagggccuuggcgcugcucaccugggccugcuugguggcgcugggcuaugugcugguguucgacgcauggacaaaggcggccugugcgugggagcaggugcccuucuuccuguucauugucuucgugguguacacacuacugcccuucagcaugcggggcgcugucgccguuggggccgucuccacugccucccaccuccuggugcucgguucuuugaugggaggcuucacgacacccaguguccggguggggcugcagcugcuggccaacgcagucaucuuccugugugggaaccugacaggcgccuuccacaagcaccaaaugcaggaugcgucccgggaccucuucaccuacacugugaagugcauccagauccgccggaagcugcgcaucgagaagcgccagcaggagaaccugcugcugucagugcuuccggcccacaucuccaugggcaugaagcuggccaucaucgaacggcucaaggagcauggugaccgucgcugcaugccugacaacaacuuccacagccucuacgucaagaggcaccagaaugucagcauccucuaugcggacaucgugggcuucacgcagcuggccagcgacuguucucccaaggagcuggugguggugcugaaugagcucuuuggcaaguucgaccagaucgccaaggccaacgagugcaugcgaaucaagauccucggcgacugcuacuacuguguaucgggccugcccgugucgcugccuacccacgcccggaacugcgugaagauggggcuggacaugugccaggccaucaagcaggugcgggaggccacgggcguggacaucaacaugcgugugggcauacacucggggaaugugcugugcggggucaucgggcugcgcaaguggcaguaugacguguggucccacgacgugucccuggccaaccggauggaggcagccggaguacccggccgggugcacaucacggaggccacgcuaaagcaccuggacaaggcguacgagguggaggaugggcgcgggcagcagcgggaacccuaccucaaggagaugaacauccgcaccuaccuggucaucgacccccggagccagcagccacccccgcccagccaacaccuccccaggcccaagggggacgcggcccugaagaugcgggcgucagugcgcaugacccgguaccucgaguccuggggggcggcacggcccuuugcacaucucaaccaccgugagagcgugagcaguggugagacccacguccccaacgggcggaggccuaagagcguuccccagcgccaccgccggaccccagacagaagcaugucccccaaggggcggucggaggaugacucguacgaugacgagaugcugucagccauugaggggcucagcuccacgaggcccugcugcuccaaguccgaugacuucuacaccuuuggguccaucuuccuggagaagggcuuugagcgcgaguaccgccuggcacccaucccccgggcccgccacgacuuugccugcgccagccugaucuucgucugcauccugcucguccauguccugcucaugcccaggacggcggcacuggguguguccuucgggcugguggccuguguacuggggcuggugcugggccugugcuuugccaccaaguucucgaggugcugcccagcucgggggacgcucugcacuaucucugagaggguggagacacagccccugcugaggcugacccuggccguccugaccaucggcagccugcucacuguggccaucaucaaccugccccugaugccuuuccaaguuccagagcugccuguuggcaaugagacaggccuacuggccgcgagcagcaagacaagagcccugugugagccccucccgcaccugcauacuguuuuuucccguuuaaaugagcucacuucauaagaaauaaaacuacagugaaaacaac

SEQ ID NO: 8 - DNA, 6196 bp, homo sapien
ugaggaacugcguguggagucagcccagucuggaugcacaggaggaugcuggcggcacagugagugaggccuggugccagagcugugcggaccccuuguuggccauggagcagcaggcccagaggcccucuccccagcccugcuugccugccucggagaggacagaggccuaggcccacgggggaggguguuggcagacagaugcccuccaggcccuggggccuccuuaacggccccuuaacgacacgcgugccaaggguggaggaugccagccaaggggcgcuacuuccucaacgagggcgaggagggcccugaccaagaugcgcucuacgagaaguaccagcucaccagccagcaugggccgcugcugcucacgcuccugcugguggccgccacugccugcguggcccucaucaucauugccuucagccagggggaccccuccagacaccaggccauucugggcauggcguuccuggugcuggcgguguuugcggcccucucugugcugauguacgucgagugucuccugcggcgcuggcucagggccuuggcgcugcucaccugggccugcuugguggcgcugggcuaugugcugguguucgacgcauggacaaaggcggccugugcgugggagcaggugcccuucuuccuguucauugucuucgugguguacacacuacugcccuucagcaugcggggcgcugucgccguuggggccgucuccacugccucccaccuccuggugcucgguucuuugaugggaggcuucacgacacccaguguccggguggggcugcagcugcuggccaacgcagucaucuuccugugugggaaccugacaggcgccuuccacaagcaccaaaugcaggaugcgucccgggaccucuucaccuacacugugaagugcauccagauccgccggaagcugcgcaucgagaagcgccagcaggagaaccugcugcugucagugcuuccggcccacaucuccaugggcaugaagcuggccaucaucgaacggcucaaggagcauggugaccgucgcugcaugccugacaacaacuuccacagccucuacgucaagaggcaccagaaugucagcauccucuaugcggacaucgugggcuucacgcagcuggccagcgacuguucucccaaggagcuggugguggugcugaaugagcucuuuggcaaguucgaccagaucgccaaggccaacgagugcaugcgaaucaagauccucggcgacugcuacuacuguguaucgggccugcccgugucgcugccuacccacgcccggaacugcgugaagauggggcuggacaugugccaggccaucaagcaggugcgggaggccacgggcguggacaucaacaugcgugugggcauacacucggggaaugugcugugcggggucaucgggcugcgcaaguggcaguaugacguguggucccacgacgugucccuggccaaccggauggaggcagccggaguacccggccgggugcacaucacggaggccacgcuaaagcaccuggacaaggcguacgagguggaggaugggcacgggcagcagcgggaacccuaccucaaggagaugaacauccgcaccuaccuggucaucgacccccggagccagcagccacccccgcccagccaacaccuccccaggcccaagggggacgcggcccugaagaugcgggcgucagugcgcaugacccgguaccucgaguccuggggggcggcacggcccuuugcacaucucaaccaccgugagagcgugagcaguggugagacccacguccccaacgggcggaggccuaagagcguuccccagcgccaccgccggaccccagacagaagcaugucccccaaggggcggucggaggaugacucguacgaugacgagaugcugucagccauugaggggcucagcuccacgaggcccugcugcuccaaguccgaugacuucuacaccuuuggguccaucuuccuggagaagggcuuugagcgcgaguaccgccuggcacccaucccccgggcccgccacgacuuugccugcgccagccugaucuucgucugcauccugcucguccauguccugcucaugcccaggacggcggcacuggguguguccuucgggcugguggccuguguacuggggcuggugcugggccugugcuuugccaccaaguucucgaggugcugcccagcucgggggacgcucugcacuaucucugagaggguggagacacagccccugcugaggcugacccuggccguccugaccaucggcagccugcucacuguggccaucaucaaccugccccugaugccuuuccaaguuccagagcugccuguuggcaaugagacaggccuacuggccgcgagcagcaagacaagagcccugugugagccccucccguacuacaccugcagcuguguccugggcuucaucgccugcucggucuuccugaggaugagccuggagccaaagguugugcugcugacaguggcccugguggccuaccuggugcucuucaaccucuccccaugcuggcagugggacugcugcggccaaggccugggcaaccucaccaagcccaacggcaccaccaguggcaccccuagcuguuccuggaaggaccugaagaccaugaccaauuucuaccugguccuguucuacaucacccugcuuacacucuccagacagauugacuauuacugccgcuuggacugccuauggaagaagaaguucaagaaggagcacgaggaguuugagaccauggagaacgugaaccgccuucuucuggagaacguccugccagcccacguggcugcccacuuuaucggugacaaguuaaacgaggacugguaccaucaguccuaugacugcgucugugucauguuugccuccgugccggacuucaaaguguucuacacagagugcgaugucaacaaagaagggcuggagugccuacgccugcucaaugagaucauugccgacuucgacgagcuccuacugaagcccaaguucagcggcguggagaagaucaagaccaucggcagcacguacauggcagcugcagggcucagcgucgccucagggcacgagaaccaggagcuggagcggcagcaugcccacauuggugucaugguggaguucagcaucgcccugaugaguaagcuggacggcaucaacaggcacuccuucaacuccuuccgccuccgcgucggcauaaaccaugggccugugauugcuggagugauuggggcccgaaaaccucaguaugacaucuggggaaacacugucaauguggccagccgaauggaaagcacuggagaacuugggaaaauccagguuaccgaggagaccugcaccauccuccagggccucggguacucuugugaaugccguggccugaucaacgucaaaggcaaaggcgagcugaggacuuacuuugucuguacggacacugccaaguuucaggggcuggggcugaacugagggcuccugcuggauuccgaaaaggccgggaagccagucuccuucccugaagcaagcccaggagaagacucuccgccccacgccaaucccaaaggcaugcagauggcugugcauguuggcuucuuuggaccugcacuggaggauuucucagacacaugcaccagauucuggcucgaagcagccacugagccauaaugcgcaggggaggccagaagcucugugccuggucuguaacaguuuccaggccagcuggagaauguucacugguucggggcugacuuugagaucuuuguucccugaggugccaggcaggcaacuuuagcacaugaugaaaacagacuuccaccucaguggccugugggcacgcacaagugaggucuguuuuucuagacaccaagggggaguaagcugagcugucuagcacggauuggagacucccucucccuggugggccuggcaaugacagcauuucucacagaggcauucugguaaaugaagcugaaagggguguuuuacaucuguaaacgguuucaaacagguagagagaaaaacaccacaauuaacacuguuacuuuuugccuugucuggcauguuuguuuuaaaugaauacauuaaugggguuuuuauccuuuugaaugacuuuucagacacuagacauaaaucucuucccuccaguguaugcucugccuuuuuaaccacugacauguaaggaggacuacugucuagcaucagcuuauggggucagcuggcuguggggauagaguccugaggaauguggucacagcaagaaggcggggagcagcagagccuugccuuugaaugaggcagcuugugaggcaagcauucuggagagaggugcuuugaaaguaaggugcggccuuucaccucuuccuugauuacucacacaucuuugcguucuccccugccguccuucaacuguaucuuacuuuucuuaccagaaaggaauggagucuguuuagagacaacuuggacaaccugugagugcaucucuucuuuccuuuagucuucacagcuaacucuggagagcuucaaaacuagaaggaucuacuccgcaugggugcaugcagaggcuccuggaucugggaagcccgcccccucacaaaugcugagccguucuugcucugaaacugcgugagucaaggcaaaugcaaaaagccagguuuuggggaugugucuuacugugcuucaacuucccaaggaauugaaagucaaccuaacuguaacaacagggugagaaaugaccaaacugcccgugacuuuuucugaauggacuucauaaccggaagacuuaaccgguggccucaucaccagagcaucgccaggauuucuaaugcacucaguuucccuacauagcagggauucuuagcuagguguccccaugaaccccguaaaguucuacacaaagucuugcauacaggagccuuuacaagaugauuauacaggguugcagauugggugacugaccagacuuguugggguccugggaugaguugccccgggcugcaaauuaagaguacagcuaagugcggggguggcgguggagggaacgaaaauugaaccugucugccugugcugugucguguggcuuuaucagcccgaggaagggcagguguauucuaauuugcacaaaggugcuggguagacuaguggcagcucucaugugcugcacauaaguggaaucaguaugaauagaagaacuugcuguauaaaggaauuucauggcaacaaugcugguaagggcaauuagccucgcuuaaguugccuuuuuuacacaccaaaacuuuuuacaugaagggcugguuucacaugaauacuauacugaaaucugugccacaccaaaacuuuuuacaugaagggcugguuucacaugaauacuauacugaaaucugugcucucaagaucuagcagugaccagggcugcccggcgggggcucuccuggcaagucaggaagguuucuguugcuaauauaacauagaaacacauuagugcacugggccucucugaggucagcauauuuguacucuuggaauauuuguuuuuuucuucaguaacaacagaaaccccaguugggaguuuaacaaauaacugacuaccacucacucaugcauuuuuauuuccaauuaaagcaaagcacugugcugugcucagauaauaauaguuuguaaguaaaaguuuuuaguuuucaguguucagguuauagaauauaacugaccauaaaaauuaccugcagguauuuucuuuuuaugaacuuguuuuuaaauuaccaaguaauuacuggugucauuuuguuuuaugacagacacacguaucuaacaaacaaacaaacagugaccuucuccaugggucaaggacuuccuuacaauuucuccugaguuaacuuuugugaaaauaauaccuaagguuuucuggcuuauugaggaaauuuccuaacaaacaaacaaacaaacaaacagaagagaagaucauuaaccacuguauacuuuguguauauaauaggucaguguaaagaaauaugauuugagguggugcaugcaaguaacuaggguuuauucuauauaaugaauauuuauagaucuguaacauuuguuucaaaaugcuguuucauuuuuauaaaguaccaguguuuagcugcuuuuuauacauuaaauuagcaauuugaaaaacuc

SEQ ID NO: 9 - DNA, 4735 bp, homo sapien
ggguguuggcagacagaugcccuccaggcccuggggccuccuuaacggccccuuaacgacacgcgugccaaggguggaggaugccagccaaggggcgcuacuuccucaacgagggcgaggagggcccugaccaagaugcgcucuacgagaaguaccagcucaccagccagcaugggccgcugcugcucacgcuccugcugguggccgccacugccugcguggcccucaucaucauugccuucagccagggggaccccuccagacaccaggccauucugggcauggcguuccuggugcuggcgguguuugcggcccucucugugcugauguacgucgagugucuccugcggcgcuggcucagggccuuggcgcugcucaccugggccugcuugguggcgcugggcuaugugcugguguucgacgcauggacaaaggcggccugugcgugggagcaggugcccuucuuccuguucauugucuucgugguguacacacuacugcccuucagcaugcggggcgcugucgccguuggggccgucuccacugccucccaccuccuggugcucgguucuuugaugggaggcuucacgacacccaguguccggguggggcugcagcugcuggccaacgcagucaucuuccugugugggaaccugacaggcgccuuccacaagcaccaaaugcaggaugcgucccgggaccucuucaccuacacugugaagugcauccagauccgccggaagcugcgcaucgagaagcgccagcaggagaaccugcugcugucagugcuuccggcccacaucuccaugggcaugaagcuggccaucaucgaacggcucaaggagcauggugaccgucgcugcaugccugacaacaacuuccacagccucuacgucaagaggcaccagaaugucagcauccucuaugcggacaucgugggcuucacgcagcuggccagcgacuguucucccaaggagcuggugguggugcugaaugagcucuuuggcaaguucgaccagaucgccaaggccaacgagugcaugcgaaucaagauccucggcgacugcuacuacuguguaucgggccugcccgugucgcugccuacccacgcccggaacugcgugaagauggggcuggacaugugccaggccaucaagcaggugcgggaggccacgggcguggacaucaacaugcgugugggcauacacucggggaaugugcugugcggggucaucgggcugcgcaaguggcaguaugacguguggucccacgacgugucccuggccaaccggauggaggcagccggaguacccggccgggugcacaucacggaggccacgcuaaagcaccuggacaaggcguacgagguggaggaugggcacgggcagcagcgggaacccuaccucaaggagaugaacauccgcaccuaccuggucaucgacccccggagccagcagccacccccgcccagccaacaccuccccaggcccaagggggacgcggcccugaagaugcgggcgucagugcgcaugacccgguaccucgaguccuggggggcggcacggcccuuugcacaucucaaccaccgugagagcgugagcaguggugagacccacguccccaacgggcggaggccuaagagcguuccccagcgccaccgccggaccccagacagaagcaugucccccaaggggcggucggaggaugacucguacgaugacgagaugcugucagccauugaggggcucagcuccacgaggcccugcugcuccaaguccgaugacuucuacaccuuuggguccaucuuccuggagaagggcuuugagcgcgaguaccgccuggcacccaucccccgggcccgccacgacuuugccugcgccagccugaucuucgucugcauccugcucguccauguccugcucaugcccaggacggcggcacuggguguguccuucgggcugguggccuguguacuggggcuggugcugggccugugcuuugccaccaaguucucgaggugcugcccagcucgggggacgcucugcacuaucucugagaggguggagacacagccccugcugaggcugacccuggccguccugaccaucggcagccugcucacuguggccaucaucaaccugccccugaugccuuuccaaguuccagagcugccuguuggcaaugagacaggccuacuggccgcgagcagcaagacaagagcccugugugagccccucccguacuacaccugcagcuguguccugggcuucaucgccugcucggucuuccugaggaugagccuggagccaaagguugugcugcugacaguggcccugguggccuaccuggugcucuucaaccucuccccaugcuggcagugggacugcugcggccaaggccugggcaaccucaccaagcccaacggcaccaccaguggcaccccuagcuguuccuggaaggaccugaagaccaugaccaauuucuaccugguccuguucuacaucacccugcuuacacucuccagacagauugacuauuacugccgcuuggacugccuauggaagaagaaguucaagaaggagcacgaggaguuugagaccauggagaacgugaaccgccuucuucuggagaacguccugccagcccacguggcugcccacuuuaucggugacaaguuaaacgaggacugguaccaucaguccuaugacugcgucugugucauguuugccuccgugccggacuucaaaguguucuacacagagugcgaugucaacaaagaagggcuggagugccuacgccugcucaaugagaucauugccgacuucgacgagcuccuacugaagcccaaguucagcggcguggagaagaucaagaccaucggcagcacguacauggcagcugcagggcucagcgucgccucagggcacgagaaccaggagcuggagcggcagcaugcccacauuggugucaugguggaguucagcaucgcccugaugaguaagcuggacggcaucaacaggcacuccuucaacuccuuccgccuccgcgucggcauaaaccaugggccugugauugcuggagugauuggggcccgaaaaccucaguaugacaucuggggaaacacugucaauguggccagccgaauggaaagcacuggagaacuugggaaaauccagguuaccgaggagaccugcaccauccuccagggccucggguacucuugugaaugccguggccugaucaacgucaaaggcaaaggcgagcugaggacuuacuuugucuguacggacacugccaaguuucaggggcuggggcugaacugagggcuccugcuggauuccgaaaaggccgggaagccagucuccuucccugaagcaagcccaggagaagacucuccgccccacgccaaucccaaaggcaugcagauggcugugcauguuggcuucuuuggaccugcacuggaggauuucucagacacaugcaccagauucuggcucgaagcagccacugagccauaaugcgcaggggaggccagaagcucugugccuggucuguaacaguuuccaggccagcuggagaauguucacugguucggggcugacuuugagaucuuuguucccugaggugccaggcaggcaacuuuagcacaugaugaaaacagacuuccaccucaguggccugugggcacgcacaagugaggucuguuuuucuagacaccaagggggaguaagcugagcugucuagcacggauuggagacucccucucccuggugggccuggcaaugacagcauuucucacagaggcauucugguaaaugaagcugaaagggguguuuuacaucuguaaacgguuucaaacagguagagagaaaaacaccacaauuaacacuguuacuuuuugccuugucuggcauguuuguuuuaaaugaauacauuaaugggguuuuuauccuuuugaaugacuuuucagacacuagacauaaaucucuucccuccaguguaugcucugccuuuuuaaccacugacauguaaggaggacuacugucuagcaucagcuuauggggucagcuggcuguggggauagaguccugaggaauguggucacagcaagaaggcggggagcagcagagccuugccuuugaaugaggcagcuugugaggcaagcauucuggagagaggugcuuugaaaguaaggugcggccuucaccucuuccuugauuacucacacaucuuugcguucuccccugccguccuucaacuguaucuuacuuuucuuaccagaaaggaauggagucuguuuagagacaacuuggacaaccugugagugcaucucuucuuuccuuuagucuucacagcuaacucuggagagcuucaaaacuagaaggaucuacuccgcaugggugcaugcagaggcuccuggaucugggaagcccgcccccucacaaaugcugagccguucuugcucugaaacugcgugagucaaggcaaaugcaaaaagccagguuuuggggaugugucuuacugugcuucaacuucccaaggaauugaaagucaaccuaacuguaacaacagggugagaaaugaccaaacugcccgugacuuuuucugaauggacuucauaaccggaagacuuaaccgguggccucaucaccagagcaucgccaggauuucuaaugcacucaguuucccuacauagcagggauucuuagcuagguguccccaugaaccccguaaa

SEQ ID NO: 10 - DNA, 3281 bp, homo sapien
gcgaucgcuuucgaaggagauagaaccaugccagccaaggggcgcuacuuccucaacgagggcgaggagggcccugaccaagaugcgcucuacgagaaguaccagcucaccagccagcaugggccgcugcugcucacgcuccugcugguggccgccacugccugcguggcccucaucaucauugccuucagccagggggaccccuccagacaccaggccauucugggcauggcguuccuggugcuggcgguguuugcggcccucucugugcugauguacgucgagugucuccugcggcgcuggcucagggccuuggcgcugcucaccugggccugcuugguggcgcugggcuaugugcugguguucgacgcauggacaaaggcggccugugcgugggagcaggugcccuucuuccuguucauugucuucgugguguacacacuacugcccuucagcaugcggggcgcugucgccguuggggccgucuccacugccucccaccuccuggugcucgguucuuugaugggaggcuucacgacacccaguguccggguggggcugcagcugcuggccaacgcagucaucuuccugugugggaaccugacaggcgccuuccacaagcaccaaaugcaggaugcgucccgggaccucuucaccuacacugugaagugcauccagauccgccggaagcugcgcaucgagaagcgccagcaggagaaccugcugcugucagugcuuccggcccacaucuccaugggcaugaagcuggccaucaucgaacggcucaaggagcauggugaccgucgcugcaugccugacaacaacuuccacagccucuacgucaagaggcaccagaaugucagcauccucuaugcggacaucgugggcuucacgcagcuggccagcgacuguucucccaaggagcuggugguggugcugaaugagcucuuuggcaaguucgaccagaucgccaaggccaacgagugcaugcgaaucaagauccucggcgacugcuacuacuguguaucgggccugcccgugucgcugccuacccacgcccggaacugcgugaagauggggcuggacaugugccaggccaucaagcaggugcgggaggccacgggcguggacaucaacaugcgugugggcauacacucggggaaugugcugugcggggucaucgggcugcgcaaguggcaguaugacguguggucccacgacgugucccuggccaaccggauggaggcagccggaguacccggccgggugcacaucacggaggccacgcuaaagcaccuggacaaggcguacgagguggaggaugggcacgggcagcagcgggaacccuaccucaaggagaugaacauccgcaccuaccuggucaucgacccccggagccagcagccacccccgcccagccaacaccuccccaggcccaagggggacgcggcccugaagaugcgggcgucagugcgcaugacccgguaccucgaguccuggggggcggcacggcccuuugcacaucucaaccaccgugagagcgugagcaguggugagacccacguccccaacgggcggaggccuaagagcguuccccagcgccaccgccggaccccagacagaagcaugucccccaaggggcggucggaggaugacucguacgaugacgagaugcugucagccauugaggggcucagcuccacgaggcccugcugcuccaaguccgaugacuucuacaccuuuggguccaucuuccuggagaagggcuuugagcgcgaguaccgccuggcacccaucccccgggcccgccacgacuuugccugcgccagccugaucuucgucugcauccugcucguccauguccugcucaugcccaggacggcggcacuggguguguccuucgggcugguggccuguguacuggggcuggugcugggccugugcuuugccaccaaguucucgaggugcugcccagcucgggggacgcucugcacuaucucugagaggguggagacacagccccugcugaggcugacccuggccguccugaccaucggcagccugcucacuguggccaucaucaaccugccccugaugccuuuccaaguuccagagcugccuguuggcaaugagacaggccuacuggccgcgagcagcaagacaagagcccugugugagccccucccguacuacaccugcagcuguguccugggcuucaucgccugcucggucuuccugaggaugagccuggagccaaagguugugcugcugacaguggcccugguggccuaccuggugcucuucaaccucuccccaugcuggcagugggacugcugcggccaaggccugggcaaccucaccaagcccaacggcaccaccaguggcaccccuagcuguuccuggaaggaccugaagaccaugaccaauuucuaccugguccuguucuacaucacccugcuuacacucuccagacagauugacuauuacugccgcuuggacugccuauggaagaagaaguucaagaaggagcacgaggaguuugagaccauggagaacgugaaccgccuucuucuggagaacguccugccagcccacguggcugcccacuuuaucggugacaaguuaaacgaggacugguaccaucaguccuaugacugcgucugugucauguuugccuccgugccggacuucaaaguguucuacacagagugcgaugucaacaaagaagggcuggagugccuacgccugcucaaugagaucauugccgacuucgacgagcuccuacugaagcccaaguucagcggcguggagaagaucaagaccaucggcagcacguacauggcagcugcagggcucagcgucgccucagggcacgagaaccaggagcuggagcggcagcaugcccacauuggugucaugguggaguucagcaucgcccugaugaguaagcuggacggcaucaacaggcacuccuucaacuccuuccgccuccgcgucggcauaaaccaugggccugugauugcuggagugauuggggcccgaaaaccucaguaugacaucuggggaaacacugucaauguggccagccgaauggaaagcacuggagaacuugggaaaauccagguuaccgaggagaccugcaccauccuccagggccucggguacucuugugaaugccguggccugaucaacgucaaaggcaaaggcgagcugaggacuuacuuugucuguacggacacugccaaguuucaggggcuggggcugaacuacguaguuuaaac

SEQ ID NO: 11 - DNA, 2496 bp, homo sapien
actctcattaggtagcgtggaaggagccttcggatggagctgaggaactgcgtgtggagtcagcccagtctggatgcacaggaggatgctggcggcacagtgagtgaggcctggtgccagagctgtgcggaccccttgttggccatggagcagcaggcccagaggccctctccccagccctgcttgcctgcctcggagaggacagaggcctaggccctggggcctccttaacggccccttaacgacacgcgtgccaagggtggaggatgccagccaaggggcgctacttcctcaacgagggcgaggagggccctgaccaagatgcgctctacgagaagtaccagctcaccagccagcatgggccgctgctgctcacgctcctgctggtggccgccactgcctgcgtggccctcatcatcattgccttcagccagggggacccctccagacaccaggccattctgggcatggcgttcctggtgctggcggtgtttgcggccctctctgtgctgatgtacgtcgagtgtctcctgcggcgctggctcagggccttggcgctgctcacctgggcctgcttggtggcgctgggctatgtgctggtgttcgacgcatggacaaaggcggcctgtgcgtgggagcaggtgcccttcttcctgttcattgtcttcgtggtgtacacactactgcccttcagcatgcggggcgctgtcgccgttggggccgtctccactgcctcccacctcctggtgctcggttctttgatgggaggcttcacgacacccagtgtccgggtggggctgcagctgctggccaacgcagtcatcttcctgtgtgggaacctgacaggcgccttccacaagcaccaaatgcaggatgcgtcccgggacctcttcacctacactgtgaagtgcatccagatccgccggaagctgcgcatcgagaagcgccagcaggagaacctgctgctgtcagtgcttccggcccacatctccatgggcatgaagctggccatcatcgaacggctcaaggagcatggtgaccgtcgctgcatgcctgacaacaacttccacagcctctacgtcaagaggcaccagaatgtcagcatcctctatgcggacatcgtgggcttcacgcagctggccagcgactgttctcccaaggagctggtggtggtgctgaatgagctctttggcaagttcgaccagatcgccaaggccaacgagtgcatgcgaatcaagatcctcggcgactgctactactgtgtatcgggcctgcccgtgtcgctgcctacccacgcccggaactgcgtgaagatggggctggacatgtgccaggccatcaagcaggtgcgggaggccacgggcgtggacatcaacatgcgtgtgggcatacactcggggaatgtgctgtgcggggtcatcgggctgcgcaagtggcagtatgacgtgtggtcccacgacgtgtccctggccaaccggatggaggcagccggagtacccggccgggtgcacatcacggaggccacgctaaagcacctggacaaggcgtacgaggtggaggatgggcgcgggcagcagcgggacccctacctcaaggagatgaacatccgcacctacctggtcatcgacccccggagccagcagccacccccgcccagccaacacctccccaggcccaagggggacgcggccctgaagatgcgggcgtcagtgcgcatgacccggtacctcgagtcctggggggcggcacggccctttgcacatctcaaccaccgtgagagcgtgagcagtggtgagacccacgtccccaacgggcggaggcctaagagcgttccccagcgccaccgccggaccccagacagaagcatgtcccccaaggggcggtcggaggatgactcgtacgatgacgagatgctgtcagccattgaggggctcagctccacgaggccctgctgctccaagtccgatgacttctacacctttgggtccatcttcctggagaagggctttgagcgcgagtaccgcctggcacccatcccccgggcccgccacgactttgcctgcgccagcctgatcttcgtctgcatcctgctcgtccatgtcctgctcatgcccaggacggcggcactgggtgtgtccttcgggctggtggcctgtgtactggggctggtgctgggcctgtgctttgccaccaagttctcgaggtgctgcccagctcgggggacgctctgcactatctctgagagggtggagacacagcccctgctgaggctgaccctggccgtcctgaccatcggcagcctgctcactgtggccatcatcaacctgcccctgatgcctttccaagttccagagctgcctgttggcaatgagacaggcctactggccgcgagcagcaagacaagagccctgtgtgagcccctcccgcacctgcatactgttttttcccgtttaaatgagctcacttcataagaaataaaactacagtgaaaacaac

SEQ ID NO: 12 - DNA, 6196 bp, homo sapien
tgaggaactgcgtgtggagtcagcccagtctggatgcacaggaggatgctggcggcacagtgagtgaggcctggtgccagagctgtgcggaccccttgttggccatggagcagcaggcccagaggccctctccccagccctgcttgcctgcctcggagaggacagaggcctaggcccacgggggagggtgttggcagacagatgccctccaggccctggggcctccttaacggccccttaacgacacgcgtgccaagggtggaggatgccagccaaggggcgctacttcctcaacgagggcgaggagggccctgaccaagatgcgctctacgagaagtaccagctcaccagccagcatgggccgctgctgctcacgctcctgctggtggccgccactgcctgcgtggccctcatcatcattgccttcagccagggggacccctccagacaccaggccattctgggcatggcgttcctggtgctggcggtgtttgcggccctctctgtgctgatgtacgtcgagtgtctcctgcggcgctggctcagggccttggcgctgctcacctgggcctgcttggtggcgctgggctatgtgctggtgttcgacgcatggacaaaggcggcctgtgcgtgggagcaggtgcccttcttcctgttcattgtcttcgtggtgtacacactactgcccttcagcatgcggggcgctgtcgccgttggggccgtctccactgcctcccacctcctggtgctcggttctttgatgggaggcttcacgacacccagtgtccgggtggggctgcagctgctggccaacgcagtcatcttcctgtgtgggaacctgacaggcgccttccacaagcaccaaatgcaggatgcgtcccgggacctcttcacctacactgtgaagtgcatccagatccgccggaagctgcgcatcgagaagcgccagcaggagaacctgctgctgtcagtgcttccggcccacatctccatgggcatgaagctggccatcatcgaacggctcaaggagcatggtgaccgtcgctgcatgcctgacaacaacttccacagcctctacgtcaagaggcaccagaatgtcagcatcctctatgcggacatcgtgggcttcacgcagctggccagcgactgttctcccaaggagctggtggtggtgctgaatgagctctttggcaagttcgaccagatcgccaaggccaacgagtgcatgcgaatcaagatcctcggcgactgctactactgtgtatcgggcctgcccgtgtcgctgcctacccacgcccggaactgcgtgaagatggggctggacatgtgccaggccatcaagcaggtgcgggaggccacgggcgtggacatcaacatgcgtgtgggcatacactcggggaatgtgctgtgcggggtcatcgggctgcgcaagtggcagtatgacgtgtggtcccacgacgtgtccctggccaaccggatggaggcagccggagtacccggccgggtgcacatcacggaggccacgctaaagcacctggacaaggcgtacgaggtggaggatgggcacgggcagcagcgggacccctacctcaaggagatgaacatccgcacctacctggtcatcgacccccggagccagcagccacccccgcccagccaacacctccccaggcccaagggggacgcggccctgaagatgcgggcgtcagtgcgcatgacccggtacctcgagtcctggggggcggcacggccctttgcacatctcaaccaccgtgagagcgtgagcagtggtgagacccacgtccccaacgggcggaggcctaagagcgttccccagcgccaccgccggaccccagacagaagcatgtcccccaaggggcggtcggaggatgactcgtacgatgacgagatgctgtcagccattgaggggctcagctccacgaggccctgctgctccaagtccgatgacttctacacctttgggtccatcttcctggagaagggctttgagcgcgagtaccgcctggcacccatcccccgggcccgccacgactttgcctgcgccagcctgatcttcgtctgcatcctgctcgtccatgtcctgctcatgcccaggacggcggcactgggtgtgtccttcgggctggtggcctgtgtactggggctggtgctgggcctgtgctttgccaccaagttctcgaggtgctgcccagctcgggggacgctctgcactatctctgagagggtggagacacagcccctgctgaggctgaccctggccgtcctgaccatcggcagcctgctcactgtggccatcatcaacctgcccctgatgcctttccaagttccagagctgcctgttggcaatgagacaggcctactggccgcgagcagcaagacaagagccctgtgtgagcccctcccgtactacacctgcagctgtgtcctgggcttcatcgcctgctcggtcttcctgaggatgagcctggagccaaaggttgtgctgctgacagtggccctggtggcctacctggtgctcttcaacctctccccatgctggcagtgggactgctgcggccaaggcctgggcaacctcaccaagcccaacggcaccaccagtggcacccctagctgttcctggaaggacctgaagaccatgaccaatttctacctggtcctgttctacatcaccctgcttacactctccagacagattgactattactgccgcttggactgcctatggaagaagaagttcaagaaggagcacgaggagtttgagaccatggagaacgtgaaccgccttcttctggagaacgtcctgccagcccacgtggctgcccactttatcggtgacaagttaaacgaggactggtaccatcagtcctatgactgcgtctgtgtcatgtttgcctccgtgccggacttcaaagtgttctacacagagtgcgatgtcaacaaagaagggctggagtgcctacgcctgctcaatgagatcattgccgacttcgacgagctcctactgaagcccaagttcagcggcgtggagaagatcaagaccatcggcagcacgtacatggcagctgcagggctcagcgtcgcctcagggcacgagaaccaggagctggagcggcagcatgcccacattggtgtcatggtggagttcagcatcgccctgatgagtaagctggacggcatcaacaggcactccttcaactccttccgcctccgcgtcggcataaaccatgggcctgtgattgctggagtgattggggcccgaaaacctcagtatgacatctggggaaacactgtcaatgtggccagccgaatggaaagcactggagaacttgggaaaatccaggttaccgaggagacctgcaccatcctccagggcctcgggtactcttgtgaatgccgtggcctgatcaacgtcaaaggcaaaggcgagctgaggacttactttgtctgtacggacactgccaagtttcaggggctggggctgaactgagggctcctgctggattccgaaaaggccgggaagccagtctccttccctgaagcaagcccaggagaagactctccgccccacgccaatcccaaaggcatgcagatggctgtgcatgttggcttctttggacctgcactggaggatttctcagacacatgcaccagattctggctcgaagcagccactgagccataatgcgcaggggaggccagaagctctgtgcctggtctgtaacagtttccaggccagctggagaatgttcactggttcggggctgactttgagatctttgttccctgaggtgccaggcaggcaactttagcacatgatgaaaacagacttccacctcagtggcctgtgggcacgcacaagtgaggtctgtttttctagacaccaagggggagtaagctgagctgtctagcacggattggagactccctctccctggtgggcctggcaatgacagcatttctcacagaggcattctggtaaatgaagctgaaaggggtgttttacatctgtaaacggtttcaaacaggtagagagaaaaacaccacaattaacactgttactttttgccttgtctggcatgtttgttttaaatgaatacattaatggggtttttatccttttgaatgacttttcagacactagacataaatctcttccctccagtgtatgctctgcctttttaaccactgacatgtaaggaggactactgtctagcatcagcttatggggtcagctggctgtggggatagagtcctgaggaatgtggtcacagcaagaaggcggggagcagcagagccttgcctttgaatgaggcagcttgtgaggcaagcattctggagagaggtgctttgaaagtaaggtgcggcctttcacctcttccttgattactcacacatctttgcgttctcccctgccgtccttcaactgtatcttacttttcttaccagaaaggaatggagtctgtttagagacaacttggacaacctgtgagtgcatctcttctttcctttagtcttcacagctaactctggagagcttcaaaactagaaggatctactccgcatgggtgcatgcagaggctcctggatctgggaagcccgccccctcacaaatgctgagccgttcttgctctgaaactgcgtgagtcaaggcaaatgcaaaaagccaggttttggggatgtgtcttactgtgcttcaacttcccaaggaattgaaagtcaacctaactgtaacaacagggtgagaaatgaccaaactgcccgtgactttttctgaatggacttcataaccggaagacttaaccggtggcctcatcaccagagcatcgccaggatttctaatgcactcagtttccctacatagcagggattcttagctaggtgtccccatgaaccccgtaaagttctacacaaagtcttgcatacaggagcctttacaagatgattatacagggttgcagattgggtgactgaccagacttgttggggtcctgggatgagttgccccgggctgcaaattaagagtacagctaagtgcgggggtggcggtggagggaacgaaaattgaacctgtctgcctgtgctgtgtcgtgtggctttatcagcccgaggaagggcaggtgtattctaatttgcacaaaggtgctgggtagactagtggcagctctcatgtgctgcacataagtggaatcagtatgaatagaagaacttgctgtataaaggaatttcatggcaacaatgctggtaagggcaattagcctcgcttaagttgccttttttacacaccaaaactttttacatgaagggctggtttcacatgaatactatactgaaatctgtgccacaccaaaactttttacatgaagggctggtttcacatgaatactatactgaaatctgtgctctcaagatctagcagtgaccagggctgcccggcgggggctctcctggcaagtcaggaaggtttctgttgctaatataacatagaaacacattagtgcactgggcctctctgaggtcagcatatttgtactcttggaatatttgtttttttcttcagtaacaacagaaaccccagttgggagtttaacaaataactgactaccactcactcatgcatttttatttccaattaaagcaaagcactgtgctgtgctcagataataatagtttgtaagtaaaagtttttagttttcagtgttcaggttatagaatataactgaccataaaaattacctgcaggtattttctttttatgaacttgtttttaaattaccaagtaattactggtgtcattttgttttatgacagacacacgtatctaacaaacaaacaaacagtgaccttctccatgggtcaaggacttccttacaatttctcctgagttaacttttgtgaaaataatacctaaggttttctggcttattgaggaaatttcctaacaaacaaacaaacaaacaaacagaagagaagatcattaaccactgtatactttgtgtatataataggtcagtgtaaagaaatatgatttgaggtggtgcatgcaagtaactagggtttattctatataatgaatatttatagatctgtaacatttgtttcaaaatgctgtttcatttttataaagtaccagtgtttagctgctttttatacattaaattagcaatttgaaaaactc

SEQ ID NO: 13 - DNA, 4735 bp, homo sapien
gggtgttggcagacagatgccctccaggccctggggcctccttaacggccccttaacgacacgcgtgccaagggtggaggatgccagccaaggggcgctacttcctcaacgagggcgaggagggccctgaccaagatgcgctctacgagaagtaccagctcaccagccagcatgggccgctgctgctcacgctcctgctggtggccgccactgcctgcgtggccctcatcatcattgccttcagccagggggacccctccagacaccaggccattctgggcatggcgttcctggtgctggcggtgtttgcggccctctctgtgctgatgtacgtcgagtgtctcctgcggcgctggctcagggccttggcgctgctcacctgggcctgcttggtggcgctgggctatgtgctggtgttcgacgcatggacaaaggcggcctgtgcgtgggagcaggtgcccttcttcctgttcattgtcttcgtggtgtacacactactgcccttcagcatgcggggcgctgtcgccgttggggccgtctccactgcctcccacctcctggtgctcggttctttgatgggaggcttcacgacacccagtgtccgggtggggctgcagctgctggccaacgcagtcatcttcctgtgtgggaacctgacaggcgccttccacaagcaccaaatgcaggatgcgtcccgggacctcttcacctacactgtgaagtgcatccagatccgccggaagctgcgcatcgagaagcgccagcaggagaacctgctgctgtcagtgcttccggcccacatctccatgggcatgaagctggccatcatcgaacggctcaaggagcatggtgaccgtcgctgcatgcctgacaacaacttccacagcctctacgtcaagaggcaccagaatgtcagcatcctctatgcggacatcgtgggcttcacgcagctggccagcgactgttctcccaaggagctggtggtggtgctgaatgagctctttggcaagttcgaccagatcgccaaggccaacgagtgcatgcgaatcaagatcctcggcgactgctactactgtgtatcgggcctgcccgtgtcgctgcctacccacgcccggaactgcgtgaagatggggctggacatgtgccaggccatcaagcaggtgcgggaggccacgggcgtggacatcaacatgcgtgtgggcatacactcggggaatgtgctgtgcggggtcatcgggctgcgcaagtggcagtatgacgtgtggtcccacgacgtgtccctggccaaccggatggaggcagccggagtacccggccgggtgcacatcacggaggccacgctaaagcacctggacaaggcgtacgaggtggaggatgggcacgggcagcagcgggacccctacctcaaggagatgaacatccgcacctacctggtcatcgacccccggagccagcagccacccccgcccagccaacacctccccaggcccaagggggacgcggccctgaagatgcgggcgtcagtgcgcatgacccggtacctcgagtcctggggggcggcacggccctttgcacatctcaaccaccgtgagagcgtgagcagtggtgagacccacgtccccaacgggcggaggcctaagagcgttccccagcgccaccgccggaccccagacagaagcatgtcccccaaggggcggtcggaggatgactcgtacgatgacgagatgctgtcagccattgaggggctcagctccacgaggccctgctgctccaagtccgatgacttctacacctttgggtccatcttcctggagaagggctttgagcgcgagtaccgcctggcacccatcccccgggcccgccacgactttgcctgcgccagcctgatcttcgtctgcatcctgctcgtccatgtcctgctcatgcccaggacggcggcactgggtgtgtccttcgggctggtggcctgtgtactggggctggtgctgggcctgtgctttgccaccaagttctcgaggtgctgcccagctcgggggacgctctgcactatctctgagagggtggagacacagcccctgctgaggctgaccctggccgtcctgaccatcggcagcctgctcactgtggccatcatcaacctgcccctgatgcctttccaagttccagagctgcctgttggcaatgagacaggcctactggccgcgagcagcaagacaagagccctgtgtgagcccctcccgtactacacctgcagctgtgtcctgggcttcatcgcctgctcggtcttcctgaggatgagcctggagccaaaggttgtgctgctgacagtggccctggtggcctacctggtgctcttcaacctctccccatgctggcagtgggactgctgcggccaaggcctgggcaacctcaccaagcccaacggcaccaccagtggcacccctagctgttcctggaaggacctgaagaccatgaccaatttctacctggtcctgttctacatcaccctgcttacactctccagacagattgactattactgccgcttggactgcctatggaagaagaagttcaagaaggagcacgaggagtttgagaccatggagaacgtgaaccgccttcttctggagaacgtcctgccagcccacgtggctgcccactttatcggtgacaagttaaacgaggactggtaccatcagtcctatgactgcgtctgtgtcatgtttgcctccgtgccggacttcaaagtgttctacacagagtgcgatgtcaacaaagaagggctggagtgcctacgcctgctcaatgagatcattgccgacttcgacgagctcctactgaagcccaagttcagcggcgtggagaagatcaagaccatcggcagcacgtacatggcagctgcagggctcagcgtcgcctcagggcacgagaaccaggagctggagcggcagcatgcccacattggtgtcatggtggagttcagcatcgccctgatgagtaagctggacggcatcaacaggcactccttcaactccttccgcctccgcgtcggcataaaccatgggcctgtgattgctggagtgattggggcccgaaaacctcagtatgacatctggggaaacactgtcaatgtggccagccgaatggaaagcactggagaacttgggaaaatccaggttaccgaggagacctgcaccatcctccagggcctcgggtactcttgtgaatgccgtggcctgatcaacgtcaaaggcaaaggcgagctgaggacttactttgtctgtacggacactgccaagtttcaggggctggggctgaactgagggctcctgctggattccgaaaaggccgggaagccagtctccttccctgaagcaagcccaggagaagactctccgccccacgccaatcccaaaggcatgcagatggctgtgcatgttggcttctttggacctgcactggaggatttctcagacacatgcaccagattctggctcgaagcagccactgagccataatgcgcaggggaggccagaagctctgtgcctggtctgtaacagtttccaggccagctggagaatgttcactggttcggggctgactttgagatctttgttccctgaggtgccaggcaggcaactttagcacatgatgaaaacagacttccacctcagtggcctgtgggcacgcacaagtgaggtctgtttttctagacaccaagggggagtaagctgagctgtctagcacggattggagactccctctccctggtgggcctggcaatgacagcatttctcacagaggcattctggtaaatgaagctgaaaggggtgttttacatctgtaaacggtttcaaacaggtagagagaaaaacaccacaattaacactgttactttttgccttgtctggcatgtttgttttaaatgaatacattaatggggtttttatccttttgaatgacttttcagacactagacataaatctcttccctccagtgtatgctctgcctttttaaccactgacatgtaaggaggactactgtctagcatcagcttatggggtcagctggctgtggggatagagtcctgaggaatgtggtcacagcaagaaggcggggagcagcagagccttgcctttgaatgaggcagcttgtgaggcaagcattctggagagaggtgctttgaaagtaaggtgcggccttcacctcttccttgattactcacacatctttgcgttctcccctgccgtccttcaactgtatcttacttttcttaccagaaaggaatggagtctgtttagagacaacttggacaacctgtgagtgcatctcttctttcctttagtcttcacagctaactctggagagcttcaaaactagaaggatctactccgcatgggtgcatgcagaggctcctggatctgggaagcccgccccctcacaaatgctgagccgttcttgctctgaaactgcgtgagtcaaggcaaatgcaaaaagccaggttttggggatgtgtcttactgtgcttcaacttcccaaggaattgaaagtcaacctaactgtaacaacagggtgagaaatgaccaaactgcccgtgactttttctgaatggacttcataaccggaagacttaaccggtggcctcatcaccagagcatcgccaggatttctaatgcactcagtttccctacatagcagggattcttagctaggtgtccccatgaaccccgtaaa

SEQ ID NO: 14 - DNA, 3281 bp, homo sapien
gcgatcgctttcgaaggagatagaaccatgccagccaaggggcgctacttcctcaacgagggcgaggagggccctgaccaagatgcgctctacgagaagtaccagctcaccagccagcatgggccgctgctgctcacgctcctgctggtggccgccactgcctgcgtggccctcatcatcattgccttcagccagggggacccctccagacaccaggccattctgggcatggcgttcctggtgctggcggtgtttgcggccctctctgtgctgatgtacgtcgagtgtctcctgcggcgctggctcagggccttggcgctgctcacctgggcctgcttggtggcgctgggctatgtgctggtgttcgacgcatggacaaaggcggcctgtgcgtgggagcaggtgcccttcttcctgttcattgtcttcgtggtgtacacactactgcccttcagcatgcggggcgctgtcgccgttggggccgtctccactgcctcccacctcctggtgctcggttctttgatgggaggcttcacgacacccagtgtccgggtggggctgcagctgctggccaacgcagtcatcttcctgtgtgggaacctgacaggcgccttccacaagcaccaaatgcaggatgcgtcccgggacctcttcacctacactgtgaagtgcatccagatccgccggaagctgcgcatcgagaagcgccagcaggagaacctgctgctgtcagtgcttccggcccacatctccatgggcatgaagctggccatcatcgaacggctcaaggagcatggtgaccgtcgctgcatgcctgacaacaacttccacagcctctacgtcaagaggcaccagaatgtcagcatcctctatgcggacatcgtgggcttcacgcagctggccagcgactgttctcccaaggagctggtggtggtgctgaatgagctctttggcaagttcgaccagatcgccaaggccaacgagtgcatgcgaatcaagatcctcggcgactgctactactgtgtatcgggcctgcccgtgtcgctgcctacccacgcccggaactgcgtgaagatggggctggacatgtgccaggccatcaagcaggtgcgggaggccacgggcgtggacatcaacatgcgtgtgggcatacactcggggaatgtgctgtgcggggtcatcgggctgcgcaagtggcagtatgacgtgtggtcccacgacgtgtccctggccaaccggatggaggcagccggagtacccggccgggtgcacatcacggaggccacgctaaagcacctggacaaggcgtacgaggtggaggatgggcacgggcagcagcgggacccctacctcaaggagatgaacatccgcacctacctggtcatcgacccccggagccagcagccacccccgcccagccaacacctccccaggcccaagggggacgcggccctgaagatgcgggcgtcagtgcgcatgacccggtacctcgagtcctggggggcggcacggccctttgcacatctcaaccaccgtgagagcgtgagcagtggtgagacccacgtccccaacgggcggaggcctaagagcgttccccagcgccaccgccggaccccagacagaagcatgtcccccaaggggcggtcggaggatgactcgtacgatgacgagatgctgtcagccattgaggggctcagctccacgaggccctgctgctccaagtccgatgacttctacacctttgggtccatcttcctggagaagggctttgagcgcgagtaccgcctggcacccatcccccgggcccgccacgactttgcctgcgccagcctgatcttcgtctgcatcctgctcgtccatgtcctgctcatgcccaggacggcggcactgggtgtgtccttcgggctggtggcctgtgtactggggctggtgctgggcctgtgctttgccaccaagttctcgaggtgctgcccagctcgggggacgctctgcactatctctgagagggtggagacacagcccctgctgaggctgaccctggccgtcctgaccatcggcagcctgctcactgtggccatcatcaacctgcccctgatgcctttccaagttccagagctgcctgttggcaatgagacaggcctactggccgcgagcagcaagacaagagccctgtgtgagcccctcccgtactacacctgcagctgtgtcctgggcttcatcgcctgctcggtcttcctgaggatgagcctggagccaaaggttgtgctgctgacagtggccctggtggcctacctggtgctcttcaacctctccccatgctggcagtgggactgctgcggccaaggcctgggcaacctcaccaagcccaacggcaccaccagtggcacccctagctgttcctggaaggacctgaagaccatgaccaatttctacctggtcctgttctacatcaccctgcttacactctccagacagattgactattactgccgcttggactgcctatggaagaagaagttcaagaaggagcacgaggagtttgagaccatggagaacgtgaaccgccttcttctggagaacgtcctgccagcccacgtggctgcccactttatcggtgacaagttaaacgaggactggtaccatcagtcctatgactgcgtctgtgtcatgtttgcctccgtgccggacttcaaagtgttctacacagagtgcgatgtcaacaaagaagggctggagtgcctacgcctgctcaatgagatcattgccgacttcgacgagctcctactgaagcccaagttcagcggcgtggagaagatcaagaccatcggcagcacgtacatggcagctgcagggctcagcgtcgcctcagggcacgagaaccaggagctggagcggcagcatgcccacattggtgtcatggtggagttcagcatcgccctgatgagtaagctggacggcatcaacaggcactccttcaactccttccgcctccgcgtcggcataaaccatgggcctgtgattgctggagtgattggggcccgaaaacctcagtatgacatctggggaaacactgtcaatgtggccagccgaatggaaagcactggagaacttgggaaaatccaggttaccgaggagacctgcaccatcctccagggcctcgggtactcttgtgaatgccgtggcctgatcaacgtcaaaggcaaaggcgagctgaggacttactttgtctgtacggacactgccaagtttcaggggctggggctgaactacgtagtttaaac

SEQ ID NO: 15 - DNA, 2496 bp, homo sapien
actctcattaggtagcgtggaaggagccttcggatggagctgaggaactgcgtgtggagtcagcccagtctggatgcacaggaggatgctggcggcacagtgagtgaggcctggtgccagagctgtgcggaccccttgttggccatggagcagcaggcccagaggccctctccccagccctgcttgcctgcctcggagaggacagaggcctaggccctggggcctccttaacggccccttaacgacacgcgtgccaagggtggaggatgccagccaaggggcgctacttcctcaacgagggcgaggagggccctgaccaagatgcgctctacgagaagtaccagctcaccagccagcatgggccgctgctgctcacgctcctgctggtggccgccactgcctgcgtggccctcatcatcattgccttcagccagggggacccctccagacaccaggccattctgggcatggcgttcctggtgctggcggtgtttgcggccctctctgtgctgatgtacgtcgagtgtctcctgcggcgctggctcagggccttggcgctgctcacctgggcctgcttggtggcgctgggctatgtgctggtgttcgacgcatggacaaaggcggcctgtgcgtgggagcaggtgcccttcttcctgttcattgtcttcgtggtgtacacactactgcccttcagcatgcggggcgctgtcgccgttggggccgtctccactgcctcccacctcctggtgctcggttctttgatgggaggcttcacgacacccagtgtccgggtggggctgcagctgctggccaacgcagtcatcttcctgtgtgggaacctgacaggcgccttccacaagcaccaaatgcaggatgcgtcccgggacctcttcacctacactgtgaagtgcatccagatccgccggaagctgcgcatcgagaagcgccagcaggagaacctgctgctgtcagtgcttccggcccacatctccatgggcatgaagctggccatcatcgaacggctcaaggagcatggtgaccgtcgctgcatgcctgacaacaacttccacagcctctacgtcaagaggcaccagaatgtcagcatcctctatgcggacatcgtgggcttcacgcagctggccagcgactgttctcccaaggagctggtggtggtgctgaatgagctctttggcaagttcgaccagatcgccaaggccaacgagtgcatgcgaatcaagatcctcggcgactgctactactgtgtatcgggcctgcccgtgtcgctgcctacccacgcccggaactgcgtgaagatggggctggacatgtgccaggccatcaagcaggtgcgggaggccacgggcgtggacatcaacatgcgtgtgggcatacactcggggaatgtgctgtgcggggtcatcgggctgcgcaagtggcagtatgacgtgtggtcccacgacgtgtccctggccaaccggatggaggcagccggagtacccggccgggtgcacatcacggaggccacgctaaagcacctggacaaggcgtacgaggtggaggatgggcgcgggcagcagcgggaaccctacctcaaggagatgaacatccgcacctacctggtcatcgacccccggagccagcagccacccccgcccagccaacacctccccaggcccaagggggacgcggccctgaagatgcgggcgtcagtgcgcatgacccggtacctcgagtcctggggggcggcacggccctttgcacatctcaaccaccgtgagagcgtgagcagtggtgagacccacgtccccaacgggcggaggcctaagagcgttccccagcgccaccgccggaccccagacagaagcatgtcccccaaggggcggtcggaggatgactcgtacgatgacgagatgctgtcagccattgaggggctcagctccacgaggccctgctgctccaagtccgatgacttctacacctttgggtccatcttcctggagaagggctttgagcgcgagtaccgcctggcacccatcccccgggcccgccacgactttgcctgcgccagcctgatcttcgtctgcatcctgctcgtccatgtcctgctcatgcccaggacggcggcactgggtgtgtccttcgggctggtggcctgtgtactggggctggtgctgggcctgtgctttgccaccaagttctcgaggtgctgcccagctcgggggacgctctgcactatctctgagagggtggagacacagcccctgctgaggctgaccctggccgtcctgaccatcggcagcctgctcactgtggccatcatcaacctgcccctgatgcctttccaagttccagagctgcctgttggcaatgagacaggcctactggccgcgagcagcaagacaagagccctgtgtgagcccctcccgcacctgcatactgttttttcccgtttaaatgagctcacttcataagaaataaaactacagtgaaaacaac

SEQ ID NO: 16 - DNA, 6196 bp, homo sapien
tgaggaactgcgtgtggagtcagcccagtctggatgcacaggaggatgctggcggcacagtgagtgaggcctggtgccagagctgtgcggaccccttgttggccatggagcagcaggcccagaggccctctccccagccctgcttgcctgcctcggagaggacagaggcctaggcccacgggggagggtgttggcagacagatgccctccaggccctggggcctccttaacggccccttaacgacacgcgtgccaagggtggaggatgccagccaaggggcgctacttcctcaacgagggcgaggagggccctgaccaagatgcgctctacgagaagtaccagctcaccagccagcatgggccgctgctgctcacgctcctgctggtggccgccactgcctgcgtggccctcatcatcattgccttcagccagggggacccctccagacaccaggccattctgggcatggcgttcctggtgctggcggtgtttgcggccctctctgtgctgatgtacgtcgagtgtctcctgcggcgctggctcagggccttggcgctgctcacctgggcctgcttggtggcgctgggctatgtgctggtgttcgacgcatggacaaaggcggcctgtgcgtgggagcaggtgcccttcttcctgttcattgtcttcgtggtgtacacactactgcccttcagcatgcggggcgctgtcgccgttggggccgtctccactgcctcccacctcctggtgctcggttctttgatgggaggcttcacgacacccagtgtccgggtggggctgcagctgctggccaacgcagtcatcttcctgtgtgggaacctgacaggcgccttccacaagcaccaaatgcaggatgcgtcccgggacctcttcacctacactgtgaagtgcatccagatccgccggaagctgcgcatcgagaagcgccagcaggagaacctgctgctgtcagtgcttccggcccacatctccatgggcatgaagctggccatcatcgaacggctcaaggagcatggtgaccgtcgctgcatgcctgacaacaacttccacagcctctacgtcaagaggcaccagaatgtcagcatcctctatgcggacatcgtgggcttcacgcagctggccagcgactgttctcccaaggagctggtggtggtgctgaatgagctctttggcaagttcgaccagatcgccaaggccaacgagtgcatgcgaatcaagatcctcggcgactgctactactgtgtatcgggcctgcccgtgtcgctgcctacccacgcccggaactgcgtgaagatggggctggacatgtgccaggccatcaagcaggtgcgggaggccacgggcgtggacatcaacatgcgtgtgggcatacactcggggaatgtgctgtgcggggtcatcgggctgcgcaagtggcagtatgacgtgtggtcccacgacgtgtccctggccaaccggatggaggcagccggagtacccggccgggtgcacatcacggaggccacgctaaagcacctggacaaggcgtacgaggtggaggatgggcacgggcagcagcgggaaccctacctcaaggagatgaacatccgcacctacctggtcatcgacccccggagccagcagccacccccgcccagccaacacctccccaggcccaagggggacgcggccctgaagatgcgggcgtcagtgcgcatgacccggtacctcgagtcctggggggcggcacggccctttgcacatctcaaccaccgtgagagcgtgagcagtggtgagacccacgtccccaacgggcggaggcctaagagcgttccccagcgccaccgccggaccccagacagaagcatgtcccccaaggggcggtcggaggatgactcgtacgatgacgagatgctgtcagccattgaggggctcagctccacgaggccctgctgctccaagtccgatgacttctacacctttgggtccatcttcctggagaagggctttgagcgcgagtaccgcctggcacccatcccccgggcccgccacgactttgcctgcgccagcctgatcttcgtctgcatcctgctcgtccatgtcctgctcatgcccaggacggcggcactgggtgtgtccttcgggctggtggcctgtgtactggggctggtgctgggcctgtgctttgccaccaagttctcgaggtgctgcccagctcgggggacgctctgcactatctctgagagggtggagacacagcccctgctgaggctgaccctggccgtcctgaccatcggcagcctgctcactgtggccatcatcaacctgcccctgatgcctttccaagttccagagctgcctgttggcaatgagacaggcctactggccgcgagcagcaagacaagagccctgtgtgagcccctcccgtactacacctgcagctgtgtcctgggcttcatcgcctgctcggtcttcctgaggatgagcctggagccaaaggttgtgctgctgacagtggccctggtggcctacctggtgctcttcaacctctccccatgctggcagtgggactgctgcggccaaggcctgggcaacctcaccaagcccaacggcaccaccagtggcacccctagctgttcctggaaggacctgaagaccatgaccaatttctacctggtcctgttctacatcaccctgcttacactctccagacagattgactattactgccgcttggactgcctatggaagaagaagttcaagaaggagcacgaggagtttgagaccatggagaacgtgaaccgccttcttctggagaacgtcctgccagcccacgtggctgcccactttatcggtgacaagttaaacgaggactggtaccatcagtcctatgactgcgtctgtgtcatgtttgcctccgtgccggacttcaaagtgttctacacagagtgcgatgtcaacaaagaagggctggagtgcctacgcctgctcaatgagatcattgccgacttcgacgagctcctactgaagcccaagttcagcggcgtggagaagatcaagaccatcggcagcacgtacatggcagctgcagggctcagcgtcgcctcagggcacgagaaccaggagctggagcggcagcatgcccacattggtgtcatggtggagttcagcatcgccctgatgagtaagctggacggcatcaacaggcactccttcaactccttccgcctccgcgtcggcataaaccatgggcctgtgattgctggagtgattggggcccgaaaacctcagtatgacatctggggaaacactgtcaatgtggccagccgaatggaaagcactggagaacttgggaaaatccaggttaccgaggagacctgcaccatcctccagggcctcgggtactcttgtgaatgccgtggcctgatcaacgtcaaaggcaaaggcgagctgaggacttactttgtctgtacggacactgccaagtttcaggggctggggctgaactgagggctcctgctggattccgaaaaggccgggaagccagtctccttccctgaagcaagcccaggagaagactctccgccccacgccaatcccaaaggcatgcagatggctgtgcatgttggcttctttggacctgcactggaggatttctcagacacatgcaccagattctggctcgaagcagccactgagccataatgcgcaggggaggccagaagctctgtgcctggtctgtaacagtttccaggccagctggagaatgttcactggttcggggctgactttgagatctttgttccctgaggtgccaggcaggcaactttagcacatgatgaaaacagacttccacctcagtggcctgtgggcacgcacaagtgaggtctgtttttctagacaccaagggggagtaagctgagctgtctagcacggattggagactccctctccctggtgggcctggcaatgacagcatttctcacagaggcattctggtaaatgaagctgaaaggggtgttttacatctgtaaacggtttcaaacaggtagagagaaaaacaccacaattaacactgttactttttgccttgtctggcatgtttgttttaaatgaatacattaatggggtttttatccttttgaatgacttttcagacactagacataaatctcttccctccagtgtatgctctgcctttttaaccactgacatgtaaggaggactactgtctagcatcagcttatggggtcagctggctgtggggatagagtcctgaggaatgtggtcacagcaagaaggcggggagcagcagagccttgcctttgaatgaggcagcttgtgaggcaagcattctggagagaggtgctttgaaagtaaggtgcggcctttcacctcttccttgattactcacacatctttgcgttctcccctgccgtccttcaactgtatcttacttttcttaccagaaaggaatggagtctgtttagagacaacttggacaacctgtgagtgcatctcttctttcctttagtcttcacagctaactctggagagcttcaaaactagaaggatctactccgcatgggtgcatgcagaggctcctggatctgggaagcccgccccctcacaaatgctgagccgttcttgctctgaaactgcgtgagtcaaggcaaatgcaaaaagccaggttttggggatgtgtcttactgtgcttcaacttcccaaggaattgaaagtcaacctaactgtaacaacagggtgagaaatgaccaaactgcccgtgactttttctgaatggacttcataaccggaagacttaaccggtggcctcatcaccagagcatcgccaggatttctaatgcactcagtttccctacatagcagggattcttagctaggtgtccccatgaaccccgtaaagttctacacaaagtcttgcatacaggagcctttacaagatgattatacagggttgcagattgggtgactgaccagacttgttggggtcctgggatgagttgccccgggctgcaaattaagagtacagctaagtgcgggggtggcggtggagggaacgaaaattgaacctgtctgcctgtgctgtgtcgtgtggctttatcagcccgaggaagggcaggtgtattctaatttgcacaaaggtgctgggtagactagtggcagctctcatgtgctgcacataagtggaatcagtatgaatagaagaacttgctgtataaaggaatttcatggcaacaatgctggtaagggcaattagcctcgcttaagttgccttttttacacaccaaaactttttacatgaagggctggtttcacatgaatactatactgaaatctgtgccacaccaaaactttttacatgaagggctggtttcacatgaatactatactgaaatctgtgctctcaagatctagcagtgaccagggctgcccggcgggggctctcctggcaagtcaggaaggtttctgttgctaatataacatagaaacacattagtgcactgggcctctctgaggtcagcatatttgtactcttggaatatttgtttttttcttcagtaacaacagaaaccccagttgggagtttaacaaataactgactaccactcactcatgcatttttatttccaattaaagcaaagcactgtgctgtgctcagataataatagtttgtaagtaaaagtttttagttttcagtgttcaggttatagaatataactgaccataaaaattacctgcaggtattttctttttatgaacttgtttttaaattaccaagtaattactggtgtcattttgttttatgacagacacacgtatctaacaaacaaacaaacagtgaccttctccatgggtcaaggacttccttacaatttctcctgagttaacttttgtgaaaataatacctaaggttttctggcttattgaggaaatttcctaacaaacaaacaaacaaacaaacagaagagaagatcattaaccactgtatactttgtgtatataataggtcagtgtaaagaaatatgatttgaggtggtgcatgcaagtaactagggtttattctatataatgaatatttatagatctgtaacatttgtttcaaaatgctgtttcatttttataaagtaccagtgtttagctgctttttatacattaaattagcaatttgaaaaactc

SEQ ID NO: 17 - DNA, 4735 bp, homo sapien
gggtgttggcagacagatgccctccaggccctggggcctccttaacggccccttaacgacacgcgtgccaagggtggaggatgccagccaaggggcgctacttcctcaacgagggcgaggagggccctgaccaagatgcgctctacgagaagtaccagctcaccagccagcatgggccgctgctgctcacgctcctgctggtggccgccactgcctgcgtggccctcatcatcattgccttcagccagggggacccctccagacaccaggccattctgggcatggcgttcctggtgctggcggtgtttgcggccctctctgtgctgatgtacgtcgagtgtctcctgcggcgctggctcagggccttggcgctgctcacctgggcctgcttggtggcgctgggctatgtgctggtgttcgacgcatggacaaaggcggcctgtgcgtgggagcaggtgcccttcttcctgttcattgtcttcgtggtgtacacactactgcccttcagcatgcggggcgctgtcgccgttggggccgtctccactgcctcccacctcctggtgctcggttctttgatgggaggcttcacgacacccagtgtccgggtggggctgcagctgctggccaacgcagtcatcttcctgtgtgggaacctgacaggcgccttccacaagcaccaaatgcaggatgcgtcccgggacctcttcacctacactgtgaagtgcatccagatccgccggaagctgcgcatcgagaagcgccagcaggagaacctgctgctgtcagtgcttccggcccacatctccatgggcatgaagctggccatcatcgaacggctcaaggagcatggtgaccgtcgctgcatgcctgacaacaacttccacagcctctacgtcaagaggcaccagaatgtcagcatcctctatgcggacatcgtgggcttcacgcagctggccagcgactgttctcccaaggagctggtggtggtgctgaatgagctctttggcaagttcgaccagatcgccaaggccaacgagtgcatgcgaatcaagatcctcggcgactgctactactgtgtatcgggcctgcccgtgtcgctgcctacccacgcccggaactgcgtgaagatggggctggacatgtgccaggccatcaagcaggtgcgggaggccacgggcgtggacatcaacatgcgtgtgggcatacactcggggaatgtgctgtgcggggtcatcgggctgcgcaagtggcagtatgacgtgtggtcccacgacgtgtccctggccaaccggatggaggcagccggagtacccggccgggtgcacatcacggaggccacgctaaagcacctggacaaggcgtacgaggtggaggatgggcacgggcagcagcgggaaccctacctcaaggagatgaacatccgcacctacctggtcatcgacccccggagccagcagccacccccgcccagccaacacctccccaggcccaagggggacgcggccctgaagatgcgggcgtcagtgcgcatgacccggtacctcgagtcctggggggcggcacggccctttgcacatctcaaccaccgtgagagcgtgagcagtggtgagacccacgtccccaacgggcggaggcctaagagcgttccccagcgccaccgccggaccccagacagaagcatgtcccccaaggggcggtcggaggatgactcgtacgatgacgagatgctgtcagccattgaggggctcagctccacgaggccctgctgctccaagtccgatgacttctacacctttgggtccatcttcctggagaagggctttgagcgcgagtaccgcctggcacccatcccccgggcccgccacgactttgcctgcgccagcctgatcttcgtctgcatcctgctcgtccatgtcctgctcatgcccaggacggcggcactgggtgtgtccttcgggctggtggcctgtgtactggggctggtgctgggcctgtgctttgccaccaagttctcgaggtgctgcccagctcgggggacgctctgcactatctctgagagggtggagacacagcccctgctgaggctgaccctggccgtcctgaccatcggcagcctgctcactgtggccatcatcaacctgcccctgatgcctttccaagttccagagctgcctgttggcaatgagacaggcctactggccgcgagcagcaagacaagagccctgtgtgagcccctcccgtactacacctgcagctgtgtcctgggcttcatcgcctgctcggtcttcctgaggatgagcctggagccaaaggttgtgctgctgacagtggccctggtggcctacctggtgctcttcaacctctccccatgctggcagtgggactgctgcggccaaggcctgggcaacctcaccaagcccaacggcaccaccagtggcacccctagctgttcctggaaggacctgaagaccatgaccaatttctacctggtcctgttctacatcaccctgcttacactctccagacagattgactattactgccgcttggactgcctatggaagaagaagttcaagaaggagcacgaggagtttgagaccatggagaacgtgaaccgccttcttctggagaacgtcctgccagcccacgtggctgcccactttatcggtgacaagttaaacgaggactggtaccatcagtcctatgactgcgtctgtgtcatgtttgcctccgtgccggacttcaaagtgttctacacagagtgcgatgtcaacaaagaagggctggagtgcctacgcctgctcaatgagatcattgccgacttcgacgagctcctactgaagcccaagttcagcggcgtggagaagatcaagaccatcggcagcacgtacatggcagctgcagggctcagcgtcgcctcagggcacgagaaccaggagctggagcggcagcatgcccacattggtgtcatggtggagttcagcatcgccctgatgagtaagctggacggcatcaacaggcactccttcaactccttccgcctccgcgtcggcataaaccatgggcctgtgattgctggagtgattggggcccgaaaacctcagtatgacatctggggaaacactgtcaatgtggccagccgaatggaaagcactggagaacttgggaaaatccaggttaccgaggagacctgcaccatcctccagggcctcgggtactcttgtgaatgccgtggcctgatcaacgtcaaaggcaaaggcgagctgaggacttactttgtctgtacggacactgccaagtttcaggggctggggctgaactgagggctcctgctggattccgaaaaggccgggaagccagtctccttccctgaagcaagcccaggagaagactctccgccccacgccaatcccaaaggcatgcagatggctgtgcatgttggcttctttggacctgcactggaggatttctcagacacatgcaccagattctggctcgaagcagccactgagccataatgcgcaggggaggccagaagctctgtgcctggtctgtaacagtttccaggccagctggagaatgttcactggttcggggctgactttgagatctttgttccctgaggtgccaggcaggcaactttagcacatgatgaaaacagacttccacctcagtggcctgtgggcacgcacaagtgaggtctgtttttctagacaccaagggggagtaagctgagctgtctagcacggattggagactccctctccctggtgggcctggcaatgacagcatttctcacagaggcattctggtaaatgaagctgaaaggggtgttttacatctgtaaacggtttcaaacaggtagagagaaaaacaccacaattaacactgttactttttgccttgtctggcatgtttgttttaaatgaatacattaatggggtttttatccttttgaatgacttttcagacactagacataaatctcttccctccagtgtatgctctgcctttttaaccactgacatgtaaggaggactactgtctagcatcagcttatggggtcagctggctgtggggatagagtcctgaggaatgtggtcacagcaagaaggcggggagcagcagagccttgcctttgaatgaggcagcttgtgaggcaagcattctggagagaggtgctttgaaagtaaggtgcggccttcacctcttccttgattactcacacatctttgcgttctcccctgccgtccttcaactgtatcttacttttcttaccagaaaggaatggagtctgtttagagacaacttggacaacctgtgagtgcatctcttctttcctttagtcttcacagctaactctggagagcttcaaaactagaaggatctactccgcatgggtgcatgcagaggctcctggatctgggaagcccgccccctcacaaatgctgagccgttcttgctctgaaactgcgtgagtcaaggcaaatgcaaaaagccaggttttggggatgtgtcttactgtgcttcaacttcccaaggaattgaaagtcaacctaactgtaacaacagggtgagaaatgaccaaactgcccgtgactttttctgaatggacttcataaccggaagacttaaccggtggcctcatcaccagagcatcgccaggatttctaatgcactcagtttccctacatagcagggattcttagctaggtgtccccatgaaccccgtaaa

SEQ ID NO: 18 - DNA, 3281 bp, homo sapien
gcgatcgctttcgaaggagatagaaccatgccagccaaggggcgctacttcctcaacgagggcgaggagggccctgaccaagatgcgctctacgagaagtaccagctcaccagccagcatgggccgctgctgctcacgctcctgctggtggccgccactgcctgcgtggccctcatcatcattgccttcagccagggggacccctccagacaccaggccattctgggcatggcgttcctggtgctggcggtgtttgcggccctctctgtgctgatgtacgtcgagtgtctcctgcggcgctggctcagggccttggcgctgctcacctgggcctgcttggtggcgctgggctatgtgctggtgttcgacgcatggacaaaggcggcctgtgcgtgggagcaggtgcccttcttcctgttcattgtcttcgtggtgtacacactactgcccttcagcatgcggggcgctgtcgccgttggggccgtctccactgcctcccacctcctggtgctcggttctttgatgggaggcttcacgacacccagtgtccgggtggggctgcagctgctggccaacgcagtcatcttcctgtgtgggaacctgacaggcgccttccacaagcaccaaatgcaggatgcgtcccgggacctcttcacctacactgtgaagtgcatccagatccgccggaagctgcgcatcgagaagcgccagcaggagaacctgctgctgtcagtgcttccggcccacatctccatgggcatgaagctggccatcatcgaacggctcaaggagcatggtgaccgtcgctgcatgcctgacaacaacttccacagcctctacgtcaagaggcaccagaatgtcagcatcctctatgcggacatcgtgggcttcacgcagctggccagcgactgttctcccaaggagctggtggtggtgctgaatgagctctttggcaagttcgaccagatcgccaaggccaacgagtgcatgcgaatcaagatcctcggcgactgctactactgtgtatcgggcctgcccgtgtcgctgcctacccacgcccggaactgcgtgaagatggggctggacatgtgccaggccatcaagcaggtgcgggaggccacgggcgtggacatcaacatgcgtgtgggcatacactcggggaatgtgctgtgcggggtcatcgggctgcgcaagtggcagtatgacgtgtggtcccacgacgtgtccctggccaaccggatggaggcagccggagtacccggccgggtgcacatcacggaggccacgctaaagcacctggacaaggcgtacgaggtggaggatgggcacgggcagcagcgggaaccctacctcaaggagatgaacatccgcacctacctggtcatcgacccccggagccagcagccacccccgcccagccaacacctccccaggcccaagggggacgcggccctgaagatgcgggcgtcagtgcgcatgacccggtacctcgagtcctggggggcggcacggccctttgcacatctcaaccaccgtgagagcgtgagcagtggtgagacccacgtccccaacgggcggaggcctaagagcgttccccagcgccaccgccggaccccagacagaagcatgtcccccaaggggcggtcggaggatgactcgtacgatgacgagatgctgtcagccattgaggggctcagctccacgaggccctgctgctccaagtccgatgacttctacacctttgggtccatcttcctggagaagggctttgagcgcgagtaccgcctggcacccatcccccgggcccgccacgactttgcctgcgccagcctgatcttcgtctgcatcctgctcgtccatgtcctgctcatgcccaggacggcggcactgggtgtgtccttcgggctggtggcctgtgtactggggctggtgctgggcctgtgctttgccaccaagttctcgaggtgctgcccagctcgggggacgctctgcactatctctgagagggtggagacacagcccctgctgaggctgaccctggccgtcctgaccatcggcagcctgctcactgtggccatcatcaacctgcccctgatgcctttccaagttccagagctgcctgttggcaatgagacaggcctactggccgcgagcagcaagacaagagccctgtgtgagcccctcccgtactacacctgcagctgtgtcctgggcttcatcgcctgctcggtcttcctgaggatgagcctggagccaaaggttgtgctgctgacagtggccctggtggcctacctggtgctcttcaacctctccccatgctggcagtgggactgctgcggccaaggcctgggcaacctcaccaagcccaacggcaccaccagtggcacccctagctgttcctggaaggacctgaagaccatgaccaatttctacctggtcctgttctacatcaccctgcttacactctccagacagattgactattactgccgcttggactgcctatggaagaagaagttcaagaaggagcacgaggagtttgagaccatggagaacgtgaaccgccttcttctggagaacgtcctgccagcccacgtggctgcccactttatcggtgacaagttaaacgaggactggtaccatcagtcctatgactgcgtctgtgtcatgtttgcctccgtgccggacttcaaagtgttctacacagagtgcgatgtcaacaaagaagggctggagtgcctacgcctgctcaatgagatcattgccgacttcgacgagctcctactgaagcccaagttcagcggcgtggagaagatcaagaccatcggcagcacgtacatggcagctgcagggctcagcgtcgcctcagggcacgagaaccaggagctggagcggcagcatgcccacattggtgtcatggtggagttcagcatcgccctgatgagtaagctggacggcatcaacaggcactccttcaactccttccgcctccgcgtcggcataaaccatgggcctgtgattgctggagtgattggggcccgaaaacctcagtatgacatctggggaaacactgtcaatgtggccagccgaatggaaagcactggagaacttgggaaaatccaggttaccgaggagacctgcaccatcctccagggcctcgggtactcttgtgaatgccgtggcctgatcaacgtcaaaggcaaaggcgagctgaggacttactttgtctgtacggacactgccaagtttcaggggctggggctgaactacgtagtttaaac

SEQ ID NO: 19 - protein, 734 aa, homo sapien [sequence omitted]

SEQ ID NO: 20 - protein, 1080 aa, homo sapien
MPAKGRYFLNEGEEGPDQDALYEKYQLTSQHGPLLLTLLLVAATACVALIIIAFSQGDPSRHQAILGMAFLVLAVFAALSVLMYVECLLRRWLRALALLTWACLVALGYVLVFDAWTKAACAWEQVPFFLFIVFVVYTLLPFSMRGAVAVGAVSTASHLLVLGSLMGGFTTPSVRVGLQLLANAVIFLCGNLTGAFHKHQMQDASRDLFTYTVKCIQIRRKLRIEKRQQENLLLSVLPAHISMGMKLAIIERLKEHGDRRCMPDNNFHSLYVKRHQNVSILYADIVGFTQLASDCSPKELVVVLNELFGKFDQIAKANECMRIKILGDCYYCVSGLPVSLPTHARNCVKMGLDMCQAIKQVREATGVDINMRVGIHSGNVLCGVIGLRKWQYDVWSHDVSLANRMEAAGVPGRVHITEATLKHLDKAYEVEDGHGQQRDPYLKEMNIRTYLVIDPRSQQPPPPSQHLPRPKGDAALKMRASVRMTRYLESWGAARPFAHLNHRESVSSGETHVPNGRRPKSVPQRHRRTPDRSMSPKGRSEDDSYDDEMLSAIEGLSSTRPCCSKSDDFYTFGSIFLEKGFEREYRLAPIPRARHDFACASLIFVCILLVHVLLMPRTAALGVSFGLVACVLGLVLGLCFATKFSRCCPARGTLCTISERVETQPLLRLTLAVLTIGSLLTVAIINLPLMPFQVPELPVGNETGLLAASSKTRALCEPLPYYTCSCVLGFIACSVFLRMSLEPKVVLLTVALVAYLVLFNLSPCWQWDCCGQGLGNLTKPNGTTSGTPSCSWKDLKTMTNFYLVLFYITLLTLSRQIDYYCRLDCLWKKKFKKEHEEFETMENVNRLLLENVLPAHVAAHFIGDKLNEDWYHQSYDCVCVMFASVPDFKVFYTECDVNKEGLECLRLLNEIIADFDELLLKPKFSGVEKIKTIGSTYMAAAGLSVASGHENQELERQHAHIGVMVEFSIALMSKLDGINRHSFNSFRLRVGINHGPVIAGVIGARKPQYDIWGNTVNVASRMESTGELGKIQVTEETCTILQGLGYSCECRGLINVKGKGELRTYFVCTDTAKFQGLGLN

SEQ ID NO: 21 - protein, 734 aa, homo sapien
MPAKGRYFLNEGEEGPDQDALYEKYQLTSQHGPLLLTLLLVAATACVALIIIAFSQGDPSRHQAILGMAFLVLAVFAALSVLMYVECLLRRWLRALALLTWACLVALGYVLVFDAWTKAACAWEQVPFFLFIVFVVYTLLPFSMRGAVAVGAVSTASHLLVLGSLMGGFTTPSVRVGLQLLANAVIFLCGNLTGAFHKHQMQDASRDLFTYTVKCIQIRRKLRIEKRQQENLLLSVLPAHISMGMKLAIIERLKEHGDRRCMPDNNFHSLYVKRHQNVSILYADIVGFTQLASDCSPKELVVVLNELFGKFDQIAKANECMRIKILGDCYYCVSGLPVSLPTHARNCVKMGLDMCQAIKQVREATGVDINMRVGIHSGNVLCGVIGLRKWQYDVWSHDVSLANRMEAAGVPGRVHITEATLKHLDKAYEVEDGRGQQREPYLKEMNIRTYLVIDPRSQQPPPPSQHLPRPKGDAALKMRASVRMTRYLESWGAARPFAHLNHRESVSSGETHVPNGRRPKSVPQRHRRTPDRSMSPKGRSEDDSYDDEMLSAIEGLSSTRPCCSKSDDFYTFGSIFLEKGFEREYRLAPIPRARHDFACASLIFVCILLVHVLLMPRTAALGVSFGLVACVLGLVLGLCFATKFSRCCPARGTLCTISERVETQPLLRLTLAVLTIGSLLTVAIINLPLMPFQVPELPVGNETGLLAASSKTRALCEPLPHLHTVFSRLNELTS

SEQ ID NO: 22 - protein, 1080 aa, homo sapien
MPAKGRYFLNEGEEGPDQDALYEKYQLTSQHGPLLLTLLLVAATACVALIIIAFSQGDPSRHQAILGMAFLVLAVFAALSVLMYVECLLRRWLRALALLTWACLVALGYVLVFDAWTKAACAWEQVPFFLFIVFVVYTLLPFSMRGAVAVGAVSTASHLLVLGSLMGGFTTPSVRVGLQLLANAVIFLCGNLTGAFHKHQMQDASRDLFTYTVKCIQIRRKLRIEKRQQENLLLSVLPAHISMGMKLAIIERLKEHGDRRCMPDNNFHSLYVKRHQNVSILYADIVGFTQLASDCSPKELVVVLNELFGKFDQIAKANECMRIKILGDCYYCVSGLPVSLPTHARNCVKMGLDMCQAIKQVREATGVDINMRVGIHSGNVLCGVIGLRKWQYDVWSHDVSLANRMEAAGVPGRVHITEATLKHLDKAYEVEDGHGQQREPYLKEMNIRTYLVIDPRSQQPPPPSQHLPRPKGDAALKMRASVRMTRYLESWGAARPFAHLNHRESVSSGETHVPNGRRPKSVPQRHRRTPDRSMSPKGRSEDDSYDDEMLSAIEGLSSTRPCCSKSDDFYTFGSIFLEKGFEREYRLAPIPRARHDFACASLIFVCILLVHVLLMPRTAALGVSFGLVACVLGLVLGLCFATKFSRCCPARGTLCTISERVETQPLLRLTLAVLTIGSLLTVAIINLPLMPFQVPELPVGNETGLLAASSKTRALCEPLPYYTCSCVLGFIACSVFLRMSLEPKVVLLTVALVAYLVLFNLSPCWQWDCCGQGLGNLTKPNGTTSGTPSCSWKDLKTMTNFYLVLFYITLLTLSRQIDYYCRLDCLWKKKFKKEHEEFETMENVNRLLLENVLPAHVAAHFIGDKLNEDWYHQSYDCVCVMFASVPDFKVFYTECDVNKEGLECLRLLNEIIADFDELLLKPKFSGVEKIKTIGSTYMAAAGLSVASGHENQELERQHAHIGVMVEFSIALMSKLDGINRHSFNSFRLRVGINHGPVIAGVIGARKPQYDIWGNTVNVASRMESTGELGKIQVTEETCTILQGLGYSCECRGLINVKGKGELRTYFVCTDTAKFQGLGLN

(19) United States
(12) Patent Application Publication
(10) Pub. No.: US 2021/0189495 A1
(43) Pub. Date: Jun. 24, 2021

(54) Adenylate Cyclase 7 (ADCY7) Variants And Uses Thereof

(71) Applicant: Regeneron Pharmaceuticals, Inc., Tarrytown, NY (US)

(72) Inventors: Jan Freudenberg, Tarrytown, NY (US); Sokol Haxhinasto, Tarrytown, NY (US)

(21) Appl. No.: 17/130,296

(22) Filed: Dec. 22, 2020

Related U.S. Application Data
(60) Provisional application No. 62/952,352, filed on Dec. 22, 2019.

(51) Int. Cl. C12Q 1/6883

(52) U.S. Cl. CPC C12Q 1/6883; C12Q 2600/158

(57) ABSTRACT

The present disclosure provides methods of treating subjects having an interferon mediated disease, methods of identifying subjects having an increased risk of developing an interferon mediated disease, and methods of detecting human Adenylate Cyclase 7 (ADCY7) variant nucleic acid molecules and variant polypeptides.

### REFERENCE TO SEQUENCE LISTING

This application includes a Sequence Listing submitted electronically as a text file named 18923803201SEQ, created on Dec. 22, 2020, with a size of 249 kilobytes. The Sequence Listing is incorporated herein by reference.

### FIELD

The present disclosure relates generally to the treatment of subjects having an interferon mediated disease, methods of identifying subjects having an increased risk of developing an interferon mediated disease, and methods of detecting ADCY7 variant nucleic acid molecules and variant polypeptides.

### BACKGROUND

Dysregulation of type I interferons (IFN-1) is a common factor in multiple diseases including autoimmune diseases. IFNs are generally classified into 3 families—IFN-1, IFN-II and IFN-III— which differ in their immunomodulatory properties, their structural homology, and the group of cells from which they are secreted. IFN-1 (IFN-α, -β, -ω, -ε, -κ) consist of the largest family and alongside IFN-III (IFN-λ) activate intracellular signaling pathways which mediate immune responses against viruses and tumors. Although most cells are capable of producing IFN-1, in most situations the majority comes from dedicated danger-sensing cells called plasmacytoid dendritic cells (pDCs). IFN-1 acts on all nucleated cells during viral invasion to inhibit viral replication. They also have potent immunostimulatory properties, including inducing the maturation and activation of myeloid dendritic cells (DCs), favoring Th1 phenotype and promote B cell activation, antibody production, and Ig class switching. These immunostimulatory properties underlie their roles in autoimmunity and in interferon-mediated diseases.

Autoimmune diseases associated with interferon dysregulation include systemic lupus erythematosus (SLE). SLE is an autoimmune disease in which common symptoms include painful and swollen joints, fever, chest pain, hair loss, mouth ulcers, swollen lymph nodes, feeling tired, and a red rash which is most commonly on the face. Often there are periods of illness, termed flares, and periods of remission during which there are few symptoms. Increased levels of serum IFN-α were described in patients with SLE over 30 years ago and were associated with disease activity and specific clinical manifestations such as fever, arthralgia, rash, and leukopenia. There is currently no cure for SLE.

Multiple sclerosis (MS) is a demyelinating autoimmune disease in which the insulating covers of nerve cells in the brain and spinal cord are damaged. This damage disrupts the ability of parts of the nervous system to communicate, resulting in a range of signs and symptoms, including physical, mental, and, sometimes, psychiatric problems. Specific symptoms can include double vision, blindness in one eye, muscle weakness, trouble with sensation, or coordination. MS takes several forms, with new symptoms either occurring in isolated attacks (relapsing forms) or building up over time (progressive forms). Between attacks, symptoms may disappear completely; however, permanent neurological problems often remain, especially with the advancement of the disease. In MS, cells from the innate and adaptive arms of the immune system cause central nervous system (CNS) inflammation. Adaptive immunity is prominent in the earlier, relapsing/remitting phase of MS (RRMS). Innate immune responses appear to underlie the later secondary progressive (SPMS) phase but are likely to contribute to brain damage at all times. There is currently no cure for MS.

Adenylate cyclase 7 (ADCY7) is one of a family of ten enzymes that convert ATP to the ubiquitous second messenger cAMP. Each has distinct tissue-specific expression patterns, with ADCY7 being expressed in haemopoietic cells, where it localizes to intracellular membranes. Cyclic AMP modulates innate and adaptive immune functions, including the inhibition of the pro-inflammatory cytokine TNFα. During inflammation, ADCY7 mediates zymosan-induced increase in intracellular cAMP, leading to protein kinase A pathway activation to modulate innate immune responses through heterotrimeric G proteins G(12/13). ADCY7 also functions in signaling cascades activated by dopamine and C5 alpha chain and mediates regulation of cAMP synthesis through the synergistic action of the stimulatory G protein with G beta:gamma complex. Further, ADCY7 functions through cAMP response regulation to keep inflammation under control during bacterial infection by sensing the presence of serum factors, such as the bioactive lysophospholipid (LPA) that regulates LPS-induced TNF-alpha production. However, ADCY7 is also required for optimal functions of B and T lymphocytes during adaptive immune responses by regulating cAMP synthesis in both B and T lymphocytes.

### SUMMARY

The present disclosure provides methods of identifying a subject having an increased risk for developing an interferon mediated disease, wherein the methods comprise: determining or having determined the presence or absence of an ADCY7 predicted loss-of-function variant nucleic acid molecule encoding a human ADCY7 polypeptide in a biological sample obtained from the subject; wherein: when the subject is ADCY7 reference, then the subject does not have an increased risk for developing interferon mediated disease; and when the subject is heterozygous for an ADCY7 predicted loss-of-function variant or homozygous for an ADCY7 predicted loss-of-function variant, then the subject has an increased risk for developing an interferon mediated disease.

The present disclosure also provides methods of treating a subject with a therapeutic agent that treats or inhibits an interferon mediated disease, wherein the subject is suffering from an interferon mediated disease, the methods comprising the steps of: determining whether the subject has an ADCY7 predicted loss-of-function variant nucleic acid molecule encoding a human ADCY7 polypeptide by: obtaining or having obtained a biological sample from the subject; and performing or having performed a genotyping assay on the biological sample to determine if the subject has a genotype comprising the ADCY7 predicted loss-of-function variant nucleic acid molecule; and when the subject is ADCY7 reference, then administering or continuing to administer to the subject the therapeutic agent that treats or inhibits the interferon mediated disease in a standard dosage amount; and when the subject is heterozygous or homozygous for an ADCY7 predicted loss-of-function variant, then administering or continuing to administer to the subject the therapeutic agent that treats or inhibits the interferon mediated disease in an amount that is the same as or greater than a standard dosage amount; wherein the presence of a genotype having the ADCY7 predicted loss-of-function variant nucleic acid molecule encoding the human ADCY7 polypeptide indicates the subject has an increased risk of developing the interferon mediated disease.

The present disclosure also provides methods of detecting a human ADCY7 variant nucleic acid molecule in a subject comprising assaying a sample obtained from the subject to determine whether a nucleic acid molecule in the sample is: i) a genomic nucleic acid molecule comprising a nucleotide sequence comprising an adenine at a position corresponding to position 34,648 according to SEQ ID NO:2, or the complement thereof; ii) an mRNA molecule comprising a nucleotide sequence comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; or iii) a cDNA molecule comprising a nucleotide sequence comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof.

The present disclosure also provides methods of detecting the presence of a human ADCY7 Asp439Glu variant polypeptide, comprising performing an assay on a sample obtained from a subject to determine whether an ADCY7 protein in the sample comprises: a glutamic acid at a position corresponding to position 439 according to SEQ ID NO:21, or a glutamic acid at a position corresponding to position 439 according to SEQ ID NO:22.

The present disclosure also provides therapeutic agents that treat or inhibit an interferon mediated disease for use in the treatment of an interferon mediated disease in a subject having: i) a genomic nucleic acid molecule having a nucleotide sequence encoding a human ADCY7 polypeptide, wherein the nucleotide sequence comprises: an adenine at a position corresponding to position 34,648 according to SEQ ID NO:2, or the complement thereof; ii) an mRNA molecule having a nucleotide sequence encoding a human ADCY7 polypeptide, wherein the nucleotide sequence comprises: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; or iii) a cDNA molecule having a nucleotide sequence encoding a human ADCY7 polypeptide, wherein the nucleotide sequence comprises: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human. In some embodiments, the subject is a human under the care of a physician.

A rare variant in the ADCY7 gene associated with an increased risk of developing an interferon mediated disease, such as SLE, multiple sclerosis, altered white blood count, and production of anti-TG and/or antinuclear antibodies, in subjects has been identified in accordance with the present disclosure. For example, a genetic alteration that changes the cytosine nucleotide of position 34,648 in the human ADCY7 reference genomic nucleic acid molecule (see, SEQ ID NO:1) to adenine has been observed to indicate that the human having such an alteration may have an increased risk of developing an interferon mediated disease, such as SLE, multiple sclerosis, altered white blood count, and production of anti-TG and antinuclear antibodies. It is believed that no variants of the ADCY7 gene or protein have any known association with multiple sclerosis. Altogether, the genetic analyses described herein surprisingly indicate that the ADCY7 gene and, in particular, a variant in the ADCY7 gene, associates with an increased risk of developing an interferon mediated disease, such as SLE, multiple sclerosis, altered white blood count, and production of anti-TG and antinuclear antibodies. Therefore, subjects that have an ADCY7 variant nucleic acid molecule or polypeptide that associates with an increased risk of developing an interferon mediated disease, such as SLE, multiple sclerosis, altered white blood count, and production of anti-TG and antinuclear antibodies, may be treated such that the interferon mediated disease is prevented, the symptoms thereof are reduced, and/or development of symptoms is repressed. Accordingly, the present disclosure provides methods of leveraging the identification of such variants in subjects to identify or stratify risk in such subjects of developing an interferon mediated disease, such as SLE, multiple sclerosis, and production of anti-TG and antinuclear autoantibodies, or to diagnose subjects as having an increased risk of developing an interferon mediated disease, such as SLE, multiple sclerosis, altered white blood count, and production of anti-TG and antinuclear antibodies, such that subjects at risk or subjects with active disease may be treated accordingly. Additionally, the present disclosure provides isolated ADCY7 variant genomic nucleic acid molecules, variant mRNA molecules, and variant cDNA molecules. Also provided herein are ADCY7 loss-of-function variant nucleic acid molecules discovered to be associated with an increased risk of developing an interferon mediated disease, such as SLE, multiple sclerosis, altered blood count, and production of autoantibodies such as anti-TG and antinuclear antibodies.

For purposes of the present disclosure, any particular human can be categorized as having one of three ADCY7 genotypes: i) ADCY7 reference; ii) heterozygous for an ADCY7 predicted loss-of-function variant; or iii) homozygous for an ADCY7 predicted loss-of-function variant. A human is ADCY7 reference when the human does not have a copy of an ADCY7 predicted loss-of-function variant nucleic acid molecule. A human is heterozygous for an ADCY7 predicted loss-of-function variant when the human has a single copy of an ADCY7 predicted loss-of-function variant nucleic acid molecule. An ADCY7 predicted loss-of-function variant nucleic acid molecule is any ADCY7 nucleic acid molecule (such as, a genomic nucleic acid molecule, an mRNA molecule, or a cDNA molecule) encoding an ADCY7 polypeptide having a partial loss-of-function, a complete loss-of-function, a predicted partial loss-of-function, or a predicted complete loss-of-function. A human who has an ADCY7 polypeptide having a partial loss-of-function (or predicted partial loss-of-function) is hypomorphic for ADCY7. The ADCY7 predicted loss-of-function variant nucleic acid molecule can be any nucleic acid molecule encoding ADCY7 Asp439Glu. A human is homozygous for an ADCY7 predicted loss-of-function variant when the human has two copies of an ADCY7 predicted loss-of-function variant nucleic acid molecule.

For subjects that are genotyped or determined to be heterozygous or homozygous for an ADCY7 predicted loss-of-function variant nucleic acid molecule, such subjects have an increased risk of developing an interferon mediated disease, such as SLE, multiple sclerosis, altered white blood count, and production of anti-TG and antinuclear antibodies. For subjects that are genotyped or determined to be heterozygous or homozygous for an ADCY7 predicted loss-of-function variant nucleic acid molecule, such subjects can be treated with an agent effective to treat an interferon mediated disease, such as SLE, multiple sclerosis, altered white blood count, and production of anti-TG and antinuclear antibodies.

In any of the embodiments described herein, the ADCY7 predicted loss-of-function variant nucleic acid molecule can be any ADCY7 nucleic acid molecule (such as, for example, genomic nucleic acid molecule, mRNA molecule, or cDNA molecule) encoding an ADCY7 polypeptide having a partial loss-of-function, a complete loss-of-function, a predicted partial loss-of-function, or a predicted complete loss-of-function. For example, the ADCY7 predicted loss-of-function variant nucleic acid molecule can be any nucleic acid molecule encoding ADCY7 Asp439Glu.

In any of the embodiments described herein, the ADCY7 predicted loss-of-function polypeptide can be any ADCY7 polypeptide having a partial loss-of-function, a complete loss-of-function, a predicted partial loss-of-function, or a predicted complete loss-of-function. In any of the embodiments described herein, the ADCY7 predicted loss-of-function polypeptide can be any of the ADCY7 polypeptides described herein including, for example, ADCY7 Asp439Glu.

Interferon mediated diseases include disorders caused by the overproduction of interferons and/or the overactivation of interferon-activated downstream genes. In any of the embodiments described herein, the interferon mediated disease is SLE, multiple sclerosis, altered white blood count, or production of anti-TG and/or antinuclear antibodies. In any of the embodiments described herein, the interferon mediated disease is SLE, or multiple sclerosis. In any of the embodiments described herein, the interferon mediated disease is SLE. In any of the embodiments described herein, the interferon mediated disease is multiple sclerosis. In any of the embodiments described herein, the interferon mediated disease is an altered white blood count. In any of the embodiments described herein, the interferon mediated disease is the production of anti-TG and/or antinuclear antibodies. Additional interferon mediated diseases include, but are not limited to, psoriasis, Sjogren's syndrome, rheumatoid arthritis, systemic sclerosis and scleroderma, inflammatory arthritis, type 1 diabetes, vitiligo, microscopic polyangiitis, granulomatosis with polyangiitis (formerly called Wegener's granulomatosis), autoimmune thyroid diseases (including Grave's disease and Hashimoto thyroiditis), juvenile idiopathic arthritis, dermatomyositis, and/or giant cell arteritis.

In some embodiments, the interferon mediated disease is not an autoimmune thyroid diseases (such as Grave's disease and Hashimoto thyroiditis), type 1 diabetes, systemic lupus erythematosus, rheumatoid arthritis, multiple sclerosis, psoriasis, or ulcerative colitis. In some embodiments, the interferon mediated disease is not an autoimmune thyroid diseases (such as Grave's disease), rheumatoid arthritis, or ulcerative colitis. In some embodiments, the interferon mediated disease is not an autoimmune thyroid diseases (such as Grave's disease and Hashimoto thyroiditis). In some embodiments, the interferon mediated disease is not type 1 diabetes. In some embodiments, the interferon mediated disease is not systemic lupus erythematosus. In some embodiments, the interferon mediated disease is not rheumatoid arthritis. In some embodiments, the interferon mediated disease is not multiple sclerosis. In some embodiments, the interferon mediated disease is not psoriasis. In some embodiments, the interferon mediated disease is not ulcerative colitis.

In some embodiments, the interferon mediated disease is not Sjogren's syndrome, systemic sclerosis, vitiligo, or juvenile idiopathic arthritis. In some embodiments, the interferon mediated disease is not Sjogren's syndrome. In some embodiments, the interferon mediated disease is not systemic sclerosis. In some embodiments, the interferon mediated disease is vitiligo. In some embodiments, the interferon mediated disease is not juvenile idiopathic arthritis.

In some embodiments, the interferon mediated disease is associated with the ADCY7 predicted loss-of-function variant nucleic acid molecule or polypeptide by a statistical significance (p value) less than 0.25, less than 0.15, or less than 0.075. In some embodiments, the interferon mediated disease is associated with the ADCY7 predicted loss-of-function variant nucleic acid molecule or polypeptide by a statistical significance (p value) less than 0.25. In some embodiments, the interferon mediated disease is associated with the ADCY7 predicted loss-of-function variant nucleic acid molecule or polypeptide by a statistical significance (p value) less than 0.15. In some embodiments, the interferon mediated disease is associated with the ADCY7 predicted loss-of-function variant nucleic acid molecule or polypeptide by a statistical significance (p value) less than 0.075.

Symptoms of an interferon mediated disease include, but are not limited to, painful and swollen joints, fever, chest pain, hair loss, mouth ulcers, swollen lymph nodes, tiredness, rash, arthralgia, leukopenia, anemia, poor ability to tolerate cold, depression, weight change, kidney failure, double vision, psychosis, vasculitis, stroke, blindness in one eye, muscle weakness, trouble with sensation, and/or trouble with coordination.

The present disclosure also provides methods of treating a subject with a therapeutic agent that treats or inhibits an interferon mediated disease, wherein the subject is suffering from an interferon mediated disease. In some embodiments, the methods comprise determining whether the subject has an ADCY7 predicted loss-of-function variant nucleic acid molecule encoding a human ADCY7 polypeptide by obtaining or having obtained a biological sample from the subject, and performing or having performed a genotyping assay on the biological sample to determine if the subject has a genotype comprising the ADCY7 predicted loss-of-function variant nucleic acid molecule. When the subject is ADCY7 reference, the therapeutic agent that treats or inhibits an interferon mediated disease is administered or continued to be administered to the subject in a standard dosage amount. When the subject is heterozygous or homozygous for an ADCY7 predicted loss-of-function variant, the therapeutic agent that treats or inhibits the interferon mediated disease is administered or continued to be administered to the subject in an amount that is the same as or greater than a standard dosage amount. The presence of a genotype having the ADCY7 predicted loss-of-function variant nucleic acid molecule encoding the human ADCY7 polypeptide indicates the subject has an increased risk of developing an interferon mediated disease. In some embodiments, the subject is ADCY7 reference. In some embodiments, the subject is heterozygous for an ADCY7 predicted loss-of-function variant. In some embodiments, the subject is homozygous for an ADCY7 predicted loss-of-function variant.

In some embodiments, the methods of treatment further comprise detecting the presence or absence of an ADCY7 predicted loss-of-function variant nucleic acid molecule encoding a human ADCY7 polypeptide in a biological sample from the subject. As used throughout the present disclosure, an “ADCY7 predicted loss-of-function variant nucleic acid molecule” is any ADCY7 nucleic acid molecule (such as, for example, genomic nucleic acid molecule, mRNA molecule, or cDNA molecule) encoding an ADCY7 polypeptide having a partial loss-of-function, a complete loss-of-function, a predicted partial loss-of-function, or a predicted complete loss-of-function.

Detecting the presence or absence of an ADCY7 predicted loss-of-function variant nucleic acid molecule in a biological sample from a subject and/or determining whether a subject has an ADCY7 predicted loss-of-function variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

The present disclosure also provides methods of treating a subject with a therapeutic agent that treats or inhibits an interferon mediated disease, wherein the subject is suffering from an interferon mediated disease. In some embodiments, the method comprises determining whether the subject has an ADCY7 predicted loss-of-function polypeptide by obtaining or having obtained a biological sample from the subject, and performing or having performed an assay on the biological sample to determine if the subject has an ADCY7 predicted loss-of-function polypeptide. When the subject does not have an ADCY7 predicted loss-of-function polypeptide, the therapeutic agent that treats or inhibits the interferon mediated disease is administered or continued to be administered to the subject in a standard dosage amount. When the subject has an ADCY7 predicted loss-of-function polypeptide, the therapeutic agent that treats or inhibits the interferon mediated disease is administered or continued to be administered to the subject in an amount that is the same as or greater than a standard dosage amount. The presence of an ADCY7 predicted loss-of-function polypeptide indicates the subject has an increased risk of developing an interferon mediated disease. In some embodiments, the subject has an ADCY7 predicted loss-of-function polypeptide. In some embodiments, the subject does not have an ADCY7 predicted loss-of-function polypeptide.

Detecting the presence or absence of an ADCY7 predicted loss-of-function polypeptide in a biological sample from a subject and/or determining whether a subject has an ADCY7 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

Examples of therapeutic agents that treat or inhibit interferon mediated diseases include, but are not limited to therapeutic agents that treat SLE, and/or multiple sclerosis.

Therapeutic agents that treat SLE include, but are not limited to PLAQUENIL® (hydroxychloroquine); DELTASONE® (prednisone); BENLYSTA® (belimumab); IMURAN® and AZASAN® (azathioprine); ASPERGUM® and FASPRIN® (aspirin); CELLCEPT® and MYFORTIC® (mycophenolate); NASACORT, ARISTOCORT, AZMACORT®, and TRIANEX (triamcinolone); DEXPAK® TAPERPAK, DXEVO®, HIDEX®, OZURDEX®, DEXPAK® 6 DAY, DEXPAK® 10 DAY, DEXPAK® 13 DAY, and MAXIDEX® (dexamethasone); ACTHAR® and H.P. ACTHAR GEL® (corticotropin); REVIMMUNE® (cyclophosphamide); and CORTONE ACETATE (cortisone).

Therapeutic agents that treat multiple sclerosis include, but are not limited to DELTASONE® (prednisone); COPAXONE® and GLATOPA® (glatiramer); GILENYA® (fingolimod); AMPYRA® (dalfampridine); REBIF®, AVONEX®, and AVONEX PEN® (interferon beta-1a); TECFIDERA® (dimethyl fumarate); DEXPAK® TAPERPAK, DXEVO®, HIDEX®, OZURDEX®, DEXPAK® 6 DAY, DEXPAK® 10 DAY, DEXPAK® 13 DAY, and MAXIDEX® (dexamethasone); TYSABRI® (natalizumab); AUBAGIO® (teriflunomide); OCREVUS® (ocrelizumab); MILLIPRED®, MILLIPRED® DP, ORAPRED ODT®, PEDIAPRED®, and VERIPRED® 20 (prednisolone); BETASERON® (interferon beta-1b); VALTREX® (valacyclovir); IMURAN® and AZASAN® (azathioprine); LEMTRADA® (alemtuzumab); MAVENCLAD® (cladribine); ACTHAR® and H.P. ACTHAR GEL® (corticotropin); REVIMMUNE® (cyclophosphamide); SOLU-MEDROL® and MEDROL® (methylprednisolone); NOVANTRONE (mitoxantrone); PLEGRIDY (peginterferon beta-1a); ZINBRYTA® (daclizumab); CELLCEPT® and MYFORTIC® (mycophenolate); MAYZENT® (siponimod); and VUMERITY® (diroximel fumarate).

In some embodiments, the dose of the therapeutic agents that treat or inhibit an interferon mediated disease can be increased by about 10%, by about 20%, by about 30%, by about 40%, by about 50%, by about 60%, by about 70%, by about 80%, or by about 90% for patients or subjects that are heterozygous or homozygous for an ADCY7 predicted loss-of-function variant (i.e., a greater amount than the standard dosage amount) compared to patients or subjects that are ADCY7 reference (who may receive a standard dosage amount). In some embodiments, the dose of the therapeutic agents that treat or inhibit an interferon mediated disease can be increased by about 10%, by about 20%, by about 30%, by about 40%, or by about 50%. In addition, the dose of therapeutic agents that treat or inhibit an interferon mediated disease in patients or subjects that are heterozygous or homozygous for an ADCY7 predicted loss-of-function variant can be administered more frequently compared to patients or subjects that are ADCY7 reference.

In some embodiments, the dose of the therapeutic agents that treat or inhibit an interferon mediated disease can be increased by about 10%, by about 20%, by about 30%, by about 40%, by about 50%, by about 60%, by about 70%, by about 80%, or by about 90% for patients or subjects that are homozygous for an ADCY7 predicted loss-of-function variant compared to patients or subjects that are heterozygous for an ADCY7 predicted loss-of-function variant. In some embodiments, the dose of the therapeutic agents that treat or inhibit an interferon mediated disease can be increased by about 10%, by about 20%, by about 30%, by about 40%, or by about 50%. In addition, the dose of therapeutic agents that treat or inhibit an interferon mediated disease in patients or subjects that are homozygous for an ADCY7 predicted loss-of-function variant can be administered more frequently compared to patients or subjects that are heterozygous for an ADCY7 predicted loss-of-function variant.

Administration of the therapeutic agents that treat or inhibit an interferon mediated disease can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the therapeutic agents that treat or inhibit an interferon mediated disease can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in an interferon mediated disease, a decrease/reduction in the severity of an interferon mediated disease (such as, for example, a reduction or inhibition of development of an interferon mediated disease), a decrease/reduction in symptoms and interferon mediated disease-related effects, delaying the onset of symptoms and interferon mediated disease-related effects, reducing the severity of symptoms of interferon mediated disease-related effects, reducing the severity of an acute episode, reducing the number of symptoms and interferon mediated disease-related effects, reducing the latency of symptoms and interferon mediated disease-related effects, an amelioration of symptoms and interferon mediated disease-related effects, reducing secondary symptoms, reducing secondary infections, preventing relapse to an interferon mediated disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of an interferon mediated disease development/progression (such as, for example, a complete or partial avoidance/inhibition or a delay), and an increased survival time of the affected host animal, following administration of a therapeutic protocol. Treatment of an interferon mediated disease encompasses the treatment of subjects already diagnosed as having any form of an interferon mediated disease at any clinical stage or manifestation, the delay of the onset or evolution or aggravation or deterioration of the symptoms or signs of an interferon mediated disease, and/or preventing and/or reducing the severity of an interferon mediated disease.

The present disclosure also provides methods of identifying a subject having an increased risk for developing an interferon mediated disease. In some embodiments, the method comprises determining or having determined in a biological sample obtained from the subject the presence or absence of an ADCY7 predicted loss-of-function variant nucleic acid molecule (such as a genomic nucleic acid molecule, mRNA molecule, and/or cDNA molecule) encoding a human ADCY7 polypeptide. When the subject lacks an ADCY7 predicted loss-of-function variant nucleic acid molecule (i.e., the subject is genotypically categorized as an ADCY7 reference), then the subject does not have an increased risk for developing an interferon mediated disease. When the subject has an ADCY7 predicted loss-of-function variant nucleic acid molecule (i.e., the subject is heterozygous for an ADCY7 predicted loss-of-function variant or homozygous for an ADCY7 predicted loss-of-function variant), then the subject has an increased risk for developing an interferon mediated disease.

Determining whether a subject has an ADCY7 predicted loss-of-function variant nucleic acid molecule in a biological sample from a subject and/or determining whether a subject has an ADCY7 predicted loss-of-function variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing an interferon mediated disease, the subject is further treated with a therapeutic agent that treats or inhibits an interferon mediated disease, as described herein. In some embodiments, when the subject is heterozygous or homozygous for an ADCY7 predicted loss-of-function variant, the subject is administered the therapeutic agent that treats or inhibits an interferon mediated disease in a dosage amount that is the same as or greater than a standard dosage amount. In some embodiments, when the subject is homozygous for an ADCY7 predicted loss-of-function variant, the subject is administered the therapeutic agent that treats or inhibits an interferon mediated disease in a dosage amount that is the same as or greater than the dosage amount administered to a subject that is heterozygous for an ADCY7 predicted loss-of-function variant. In some embodiments, the subject is ADCY7 reference. In some embodiments, the subject is heterozygous for an ADCY7 predicted loss-of-function variant. In some embodiments, the subject is homozygous for an ADCY7 predicted loss-of-function variant.

The present disclosure also provides methods of detecting the presence or absence of an ADCY7 predicted loss-of-function variant genomic nucleic acid molecule in a biological sample from a subject, and/or an ADCY7 predicted loss-of-function variant mRNA molecule in a biological sample from a subject, and/or an ADCY7 predicted loss-of-function variant cDNA molecule produced from an mRNA molecule in a biological sample from a subject. It is understood that gene sequences within a population and mRNA molecules encoded by such genes can vary due to polymorphisms such as single-nucleotide polymorphisms. The sequences provided herein for the ADCY7 variant genomic nucleic acid molecule, ADCY7 variant mRNA molecule, and ADCY7 variant cDNA molecule are only exemplary sequences. Other sequences for the ADCY7 variant genomic nucleic acid molecule, variant mRNA molecule, and variant cDNA molecule are also possible.

The biological sample can be derived from any cell, tissue, or biological fluid from the subject. The sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The sample used in the methods disclosed herein will vary based on the assay format, nature of the detection method, and the tissues, cells, or extracts that are used as the sample. A biological sample can be processed differently depending on the assay being employed. For example, when detecting any ADCY7 variant nucleic acid molecule, preliminary processing designed to isolate or enrich the sample for the genomic DNA can be employed. A variety of techniques may be used for this purpose. When detecting the level of any ADCY7 variant mRNA, different techniques can be used enrich the biological sample with mRNA. Various methods to detect the presence or level of an mRNA or the presence of a particular variant genomic DNA locus can be used.

In some embodiments, detecting a human ADCY7 predicted loss-of-function variant nucleic acid molecule in a subject comprises assaying or genotyping a biological sample obtained from the subject to determine whether an ADCY7 genomic nucleic acid molecule in the biological sample, and/or an ADCY7 mRNA molecule in the biological sample, and/or an ADCY7 cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an ADCY7 predicted loss-of-function variant nucleic acid molecule (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the nucleotide sequence comprises: an adenine at a position corresponding to position 34,648 according to SEQ ID NO:2 (for genomic nucleic acid molecules); an adenine at a position corresponding to position 1,583 according to SEQ ID NO:7 (for mRNA molecules); or an adenine at a position corresponding to position 1,583 according to SEQ ID NO:15 (for cDNA molecules obtained from mRNA molecules).

In some embodiments, the nucleotide sequence comprises: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof.

In some embodiments, the nucleotide sequence comprises: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof.

In some embodiments, the biological sample comprises a cell or cell lysate. Such methods can further comprise, for example, obtaining a biological sample from the subject comprising an ADCY7 genomic nucleic acid molecule or mRNA molecule, and if mRNA, optionally reverse transcribing the mRNA into cDNA. Such assays can comprise, for example determining the identity of these positions of the particular ADCY7 nucleic acid molecule. In some embodiments, the method is an in vitro method.

In some embodiments, the determining step, detecting step, or genotyping assay comprises sequencing at least a portion of the nucleotide sequence of the ADCY7 genomic nucleic acid molecule, the ADCY7 mRNA molecule, or the ADCY7 cDNA molecule in the biological sample, wherein the sequenced portion comprises one or more variations that cause a loss-of-function (partial or complete) or are predicted to cause a loss-of-function (partial or complete).

In some embodiments, the determining step, detecting step, or genotyping assay comprises sequencing at least a portion of: the nucleotide sequence of the ADCY7 genomic nucleic acid molecule in the biological sample, wherein the sequenced portion comprises a position corresponding to position 34,648 according to SEQ ID NO:2, or the complement thereof; the nucleotide sequence of the ADCY7 mRNA molecule in the biological sample, wherein the sequenced portion comprises a position corresponding to position 1,583 according to SEQ ID NO:7, or the complement thereof; and/or the nucleotide sequence of the ADCY7 cDNA molecule produced from the mRNA in the biological sample, wherein the sequenced portion comprises a position corresponding to position 1,583 according to SEQ ID NO:15, or the complement thereof. When the sequenced portion of the ADCY7 nucleic acid molecule in the biological sample comprises: an adenine at a position corresponding to position 34,648 according to SEQ ID NO:2, an adenine at a position corresponding to position 1,583 according to SEQ ID NO:7, or an adenine at a position corresponding to position 1,583 according to SEQ ID NO:15, then the ADCY7 nucleic acid molecule in the biological sample is an ADCY7 predicted loss-of-function variant nucleic acid molecule.

In some embodiments, the determining step, detecting step, or genotyping assay comprises sequencing at least a portion of: the nucleotide sequence of the ADCY7 mRNA molecule in the biological sample, wherein the sequenced portion comprises a position corresponding to position 1,582 according to SEQ ID NO:8, or the complement thereof; and/or the nucleotide sequence of the ADCY7 cDNA molecule produced from the mRNA in the biological sample, wherein the sequenced portion comprises a position corresponding to position 1,582 according to SEQ ID NO:16, or the complement thereof. When the sequenced portion of the ADCY7 nucleic acid molecule in the biological sample comprises: an adenine at a position corresponding to position 1,582 according to SEQ ID NO:8, or an adenine at a position corresponding to position 1,582 according to SEQ ID NO:16, then the ADCY7 nucleic acid molecule in the biological sample is an ADCY7 predicted loss-of-function variant nucleic acid molecule.

In some embodiments, the determining step, detecting step, or genotyping assay comprises sequencing at least a portion of: the nucleotide sequence of the ADCY7 mRNA molecule in the biological sample, wherein the sequenced portion comprises a position corresponding to position 1,397 according to SEQ ID NO:9, or the complement thereof; and/or the nucleotide sequence of the ADCY7 cDNA molecule produced from the mRNA in the biological sample, wherein the sequenced portion comprises a position corresponding to position 1,397 according to SEQ ID NO:17, or the complement thereof. When the sequenced portion of the ADCY7 nucleic acid molecule in the biological sample comprises: an adenine at a position corresponding to position 1,397 according to SEQ ID NO:9, or an adenine at a position corresponding to position 1,397 according to SEQ ID NO:17, then the ADCY7 nucleic acid molecule in the biological sample is an ADCY7 predicted loss-of-function variant nucleic acid molecule.

In some embodiments, the determining step, detecting step, or genotyping assay comprises sequencing at least a portion of: the nucleotide sequence of the ADCY7 mRNA molecule in the biological sample, wherein the sequenced portion comprises a position corresponding to position 1,344 according to SEQ ID NO:10, or the complement thereof; and/or the nucleotide sequence of the ADCY7 cDNA molecule produced from the mRNA in the biological sample, wherein the sequenced portion comprises a position corresponding to position 1,344 according to SEQ ID NO:18, or the complement thereof. When the sequenced portion of the ADCY7 nucleic acid molecule in the biological sample comprises: an adenine at a position corresponding to position 1,344 according to SEQ ID NO:10, or an adenine at a position corresponding to position 1,344 according to SEQ ID NO:18, then the ADCY7 nucleic acid molecule in the biological sample is an ADCY7 predicted loss-of-function variant nucleic acid molecule.

In some embodiments, the determining step, detecting step, or genotyping assay comprises sequencing at least a portion of the nucleotide sequence of the ADCY7 genomic nucleic acid molecule in the biological sample, wherein the sequenced portion comprises a position corresponding to position 34,648 according to SEQ ID NO:2, or the complement thereof. When the sequenced portion of the ADCY7 nucleic acid molecule in the biological sample comprises an adenine at a position corresponding to position 34,648 according to SEQ ID NO:2, then the ADCY7 nucleic acid molecule in the biological sample is an ADCY7 predicted loss-of-function variant nucleic acid molecule.

In some embodiments, the determining step, detecting step, or genotyping assay comprises sequencing at least a portion of the nucleotide sequence of the ADCY7 mRNA molecule in the biological sample, wherein the sequenced portion comprises a position corresponding to: position 1,583 according to SEQ ID NO:7, or the complement thereof; position 1,582 according to SEQ ID NO:8, or the complement thereof; position 1,397 according to SEQ ID NO:9, or the complement thereof; position 1,344 according to SEQ ID NO:10, or the complement thereof. When the sequenced portion of the ADCY7 nucleic acid molecule in the biological sample comprises: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10; then the ADCY7 nucleic acid molecule in the biological sample is an ADCY7 predicted loss-of-function variant nucleic acid molecule.

In some embodiments, the determining step, detecting step, or genotyping assay comprises sequencing at least a portion of the nucleotide sequence of the ADCY7 cDNA molecule in the biological sample, wherein the sequenced portion comprises a position corresponding to: position 1,583 according to SEQ ID NO:15, or the complement thereof; position 1,582 according to SEQ ID NO:16, or the complement thereof; position 1,397 according to SEQ ID NO:17, or the complement thereof; or position 1,333 according to SEQ ID NO:18, or the complement thereof. When the sequenced portion of the ADCY7 nucleic acid molecule in the biological sample comprises: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18; then the ADCY7 nucleic acid molecule in the biological sample is an ADCY7 predicted loss-of-function variant nucleic acid molecule.

In some embodiments, the determining step, detecting step, or genotyping assay comprises: a) contacting the biological sample with a primer hybridizing to a portion of the nucleotide sequence of the ADCY7: genomic nucleic acid molecule that is proximate to a position corresponding to position 34,648 according to SEQ ID NO:2; mRNA molecule that is proximate to a position corresponding to position 1,583 according to SEQ ID NO:7; and/or cDNA molecule that is proximate to a position corresponding to position 1,583 according to SEQ ID NO:15; b) extending the primer at least through the position of the nucleotide sequence of the ADCY7: genomic nucleic acid molecule corresponding to position 34,648 according to SEQ ID NO:2; mRNA molecule corresponding to position 1,583 according to SEQ ID NO:7; and/or cDNA molecule corresponding to position 1,583 according to SEQ ID NO:15; and c) determining whether the extension product of the primer comprises: an adenine at a position corresponding to position 34,648 according to SEQ ID NO:2; an adenine at a position corresponding to position 1,583 according to SEQ ID NO:7; and/or an adenine at a position corresponding to position 1,583 according to SEQ ID NO:15.

In some embodiments, the determining step, detecting step, or genotyping assay comprises: a) contacting the biological sample with a primer hybridizing to a portion of the nucleotide sequence of the ADCY7: mRNA molecule that is proximate to a position corresponding to position 1,582 according to SEQ ID NO:8; and/or cDNA molecule that is proximate to a position corresponding to position 1,582 according to SEQ ID NO:16; b) extending the primer at least through the position of the nucleotide sequence of the ADCY7: mRNA molecule corresponding to position 1,582 according to SEQ ID NO:8; and/or cDNA molecule corresponding to position 1,582 according to SEQ ID NO:16; and c) determining whether the extension product of the primer comprises: an adenine at a position corresponding to position 1,582 according to SEQ ID NO:8; and/or an adenine at a position corresponding to position 1,582 according to SEQ ID NO:16.

In some embodiments, the determining step, detecting step, or genotyping assay comprises: a) contacting the biological sample with a primer hybridizing to a portion of the nucleotide sequence of the ADCY7: mRNA molecule that is proximate to a position corresponding to position 1,397 according to SEQ ID NO:9; and/or cDNA molecule that is proximate to a position corresponding to position 1,397 according to SEQ ID NO:17; b) extending the primer at least through the position of the nucleotide sequence of the ADCY7: mRNA molecule corresponding to position 1,397 according to SEQ ID NO:9; and/or cDNA molecule corresponding to position 1,397 according to SEQ ID NO:17; and c) determining whether the extension product of the primer comprises: an adenine at a position corresponding to position 1,397 according to SEQ ID NO:9; and/or an adenine at a position corresponding to position 1,397 according to SEQ ID NO:17.

In some embodiments, the determining step, detecting step, or genotyping assay comprises: a) contacting the biological sample with a primer hybridizing to a portion of the nucleotide sequence of the ADCY7: mRNA molecule that is proximate to a position corresponding to position 1,344 according to SEQ ID NO:10; and/or cDNA molecule that is proximate to a position corresponding to position 1,344 according to SEQ ID NO:18; b) extending the primer at least through the position of the nucleotide sequence of the ADCY7: mRNA molecule corresponding to position 1,344 according to SEQ ID NO:10; and/or cDNA molecule corresponding to position 1,344 according to SEQ ID NO:18; and c) determining whether the extension product of the primer comprises: an adenine at a position corresponding to position 1,344 according to SEQ ID NO:10; and/or an adenine at a position corresponding to position 1,344 according to SEQ ID NO:18.

In some embodiments, the determining step, detecting step, or genotyping assay comprises: a) contacting the biological sample with a primer hybridizing to a portion of the nucleotide sequence of the ADCY7 genomic nucleic acid molecule that is proximate to a position corresponding to position 34,648 according to SEQ ID NO:2; b) extending the primer at least through the position of the nucleotide sequence of the ADCY7 genomic nucleic acid molecule corresponding to position 34,648 according to SEQ ID NO:2; and c) determining whether the extension product of the primer comprises an adenine at a position corresponding to position 34,648 according to SEQ ID NO:2.

In some embodiments, the determining step, detecting step, or genotyping assay comprises: a) contacting the biological sample with a primer hybridizing to a portion of the nucleotide sequence of the ADCY7 mRNA molecule that is proximate to a position corresponding to: position 1,583 according to SEQ ID NO:7; position 1,582 according to SEQ ID NO:8; position 1,397 according to SEQ ID NO:9; position 1,344 according to SEQ ID NO:10; b) extending the primer at least through the position of the nucleotide sequence of the ADCY7 mRNA molecule corresponding to: position 1,583 according to SEQ ID NO:7; position 1,582 according to SEQ ID NO:8; position 1,397 according to SEQ ID NO:9; position 1,344 according to SEQ ID NO:10; and c) determining whether the extension product of the primer comprises: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10.

In some embodiments, the determining step, detecting step, or genotyping assay comprises: a) contacting the biological sample with a primer hybridizing to a portion of the nucleotide sequence of the ADCY7 cDNA molecule that is proximate to a position corresponding to: position 1,583 according to SEQ ID NO:15; position 1,582 according to SEQ ID NO:16; position 1,397 according to SEQ ID NO:17; position 1,344 according to SEQ ID NO:18; b) extending the primer at least through the position of the nucleotide sequence of the ADCY7 cDNA molecule corresponding to: position 1,583 according to SEQ ID NO:15; position 1,582 according to SEQ ID NO:16; position 1,397 according to SEQ ID NO:17; position 1,344 according to SEQ ID NO:18; and c) determining whether the extension product of the primer comprises: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18.

In some embodiments, the assay comprises sequencing the entire nucleic acid molecule. In some embodiments, only an ADCY7 genomic nucleic acid molecule is analyzed. In some embodiments, only an ADCY7 mRNA is analyzed. In some embodiments, only an ADCY7 cDNA obtained from ADCY7 mRNA is analyzed.

In some embodiments, the determining step, detecting step, or genotyping assay comprises: a) amplifying at least a portion of the nucleic acid molecule that encodes the human ADCY7 polypeptide, wherein the amplified portion comprises: an adenine at a position corresponding to position 34,648 according to SEQ ID NO:2, or the complement thereof; an adenine at a position corresponding to position 1,583 according to SEQ ID NO:7, or the complement thereof; and/or an adenine at a position corresponding to position 1,583 according to SEQ ID NO:15, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 34,648 according to SEQ ID NO:2, or the complement thereof; an adenine at a position corresponding to position 1,583 according to SEQ ID NO:7, or the complement thereof; and/or an adenine at a position corresponding to position 1,583 according to SEQ ID NO:15, or the complement thereof; and d) detecting the detectable label.

In some embodiments, the determining step, detecting step, or genotyping assay comprises: a) amplifying at least a portion of the nucleic acid molecule that encodes the human ADCY7 polypeptide, wherein the amplified portion comprises: an adenine at a position corresponding to position 1,582 according to SEQ ID NO:8, or the complement thereof; or an adenine at a position corresponding to position 1,582 according to SEQ ID NO:16, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,582 according to SEQ ID NO:8, or the complement thereof; or an adenine at a position corresponding to position 1,582 according to SEQ ID NO:16, or the complement thereof; and d) detecting the detectable label.

In some embodiments, the determining step, detecting step, or genotyping assay comprises: a) amplifying at least a portion of the nucleic acid molecule that encodes the human ADCY7 polypeptide, wherein the amplified portion comprises: an adenine at a position corresponding to position 1,397 according to SEQ ID NO:9, or the complement thereof; or an adenine at a position corresponding to position 1,397 according to SEQ ID NO:17, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,397 according to SEQ ID NO:9, or the complement thereof; or an adenine at a position corresponding to position 1,397 according to SEQ ID NO:17, or the complement thereof; and d) detecting the detectable label.

In some embodiments, the determining step, detecting step, or genotyping assay comprises: a) amplifying at least a portion of the nucleic acid molecule that encodes the human ADCY7 polypeptide, wherein the amplified portion comprises: an adenine at a position corresponding to position 1,344 according to SEQ ID NO:10, or the complement thereof; or an adenine at a position corresponding to position 1,344 according to SEQ ID NO:18, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,344 according to SEQ ID NO:10, or the complement thereof; or an adenine at a position corresponding to position 1,344 according to SEQ ID NO:18, or the complement thereof; and d) detecting the detectable label.

In some embodiments, the determining step, detecting step, or genotyping assay comprises: a) amplifying at least a portion of the nucleic acid molecule that encodes the human ADCY7 polypeptide, wherein the amplified portion comprises: an adenine at a position corresponding to position 34,648 according to SEQ ID NO:2, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 34,648 according to SEQ ID NO:2, or the complement thereof; and d) detecting the detectable label.

In some embodiments, the determining step, detecting step, or genotyping assay comprises: a) amplifying at least a portion of the nucleic acid molecule that encodes the human ADCY7 polypeptide, wherein the amplified portion comprises: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; and d) detecting the detectable label.

In some embodiments, the determining step, detecting step, or genotyping assay comprises: a) amplifying at least a portion of the nucleic acid molecule that encodes the human ADCY7 polypeptide, wherein the amplified portion comprises: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: an adenine at a position corresponding to position 34,648 according to SEQ ID NO:2, or the complement thereof; an adenine at a position corresponding to position 1,583 according to SEQ ID NO:7, or the complement thereof; or an adenine at a position corresponding to position 1,583 according to SEQ ID NO:15, or the complement thereof; and detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: an adenine at a position corresponding to position 1,582 according to SEQ ID NO:8, or the complement thereof; or an adenine at a position corresponding to position 1,582 according to SEQ ID NO:16, or the complement thereof; and detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: an adenine at a position corresponding to position 1,397 according to SEQ ID NO:9, or the complement thereof; or an adenine at a position corresponding to position 1,397 according to SEQ ID NO:17, or the complement thereof; and detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: an adenine at a position corresponding to position 1,344 according to SEQ ID NO:10, or the complement thereof; or an adenine at a position corresponding to position 1,344 according to SEQ ID NO:18, or the complement thereof; and detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising an adenine at a position corresponding to position 34,648 according to SEQ ID NO:2, or the complement thereof; and detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; and detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; and detecting the detectable label.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an ADCY7 variant genomic sequence, variant mRNA sequence, or variant cDNA sequence and not the corresponding ADCY7 reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an ADCY7 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether an ADCY7 nucleic acid molecule (genomic nucleic acid molecule, mRNA molecule, or cDNA molecule), or complement thereof, within a biological sample comprises a nucleotide sequence comprising an adenine at a position corresponding to position 34,648 according to SEQ ID NO:2 (genomic nucleic acid molecule), or an adenine at a position corresponding to position 1,583 according to SEQ ID NO:7 (mRNA molecule), or an adenine at a position corresponding to position 1,583 according to SEQ ID NO:15 (cDNA molecule), the biological sample can be subjected to an amplification method using a primer pair that includes a first primer derived from the 5′ flanking sequence adjacent to an adenine at a position corresponding to position 34,648 according to SEQ ID NO:2, or an adenine at a position corresponding to position 1,583 according to SEQ ID NO:7, or an adenine at a position corresponding to position 1,583 according to SEQ ID NO:15, and a second primer derived from the 3′ flanking sequence adjacent to an adenine at a position corresponding to position 34,648 according to SEQ ID NO:2, or an adenine at a position corresponding to position 1,583 according to SEQ ID NO:7, or an adenine at a position corresponding to position 1,583 according to SEQ ID NO:15 to produce an amplicon that is indicative of the presence of the SNP at positions encoding an adenine at a position corresponding to position 34,648 according to SEQ ID NO:2, or an adenine at a position corresponding to position 1,583 according to SEQ ID NO:7, or an adenine at a position corresponding to position 1,583 according to SEQ ID NO:15. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising an adenine at a position corresponding to position 34,648 according to SEQ ID NO:2, or an adenine at a position corresponding to position 1,583 according to SEQ ID NO:7, or an adenine at a position corresponding to position 1,583 according to SEQ ID NO:15, and at least 1, 2, 3, 4, 5, 6, 7, 8, 9, 10, or more nucleotides on each side of positions comprising an adenine at a position corresponding to position 34,648 according to SEQ ID NO:2, or an adenine at a position corresponding to position 1,583 according to SEQ ID NO:7, or an adenine at a position corresponding to position 1,583 according to SEQ ID NO:15.

In some embodiments, to determine whether an ADCY7 nucleic acid molecule (genomic nucleic acid molecule, mRNA molecule, or cDNA molecule), or complement thereof, within a biological sample comprises a nucleotide sequence comprising an adenine at a position corresponding to position 1,582 according to SEQ ID NO:8 (mRNA molecule), or an adenine at a position corresponding to position 1,582 according to SEQ ID NO:16 (cDNA molecule), the biological sample can be subjected to an amplification method using a primer pair that includes a first primer derived from the 5′ flanking sequence adjacent to an adenine at a position corresponding to position 1,582 according to SEQ ID NO:8, or an adenine at a position corresponding to position 1,582 according to SEQ ID NO:16, and a second primer derived from the 3′ flanking sequence adjacent to an adenine at a position corresponding to position 1,582 according to SEQ ID NO:8, or an adenine at a position corresponding to position 1,582 according to SEQ ID NO:16 to produce an amplicon that is indicative of the presence of the SNP at positions encoding an adenine at a position corresponding to position 1,582 according to SEQ ID NO:8, or an adenine at a position corresponding to position 1,582 according to SEQ ID NO:16. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising an adenine at a position corresponding to position 1,582 according to SEQ ID NO:8, or an adenine at a position corresponding to position 1,582 according to SEQ ID NO:16, and at least 1, 2, 3, 4, 5, 6, 7, 8, 9, 10, or more nucleotides on each side of positions comprising an adenine at a position corresponding to position 1,582 according to SEQ ID NO:8, or an adenine at a position corresponding to position 1,582 according to SEQ ID NO:16.

In some embodiments, to determine whether an ADCY7 nucleic acid molecule (genomic nucleic acid molecule, mRNA molecule, or cDNA molecule), or complement thereof, within a biological sample comprises a nucleotide sequence comprising an adenine at a position corresponding to position 1,397 according to SEQ ID NO:9 (mRNA molecule), or an adenine at a position corresponding to position 1,397 according to SEQ ID NO:17 (cDNA molecule), the biological sample can be subjected to an amplification method using a primer pair that includes a first primer derived from the 5′ flanking sequence adjacent to an adenine at a position corresponding to position 1,397 according to SEQ ID NO:9, or an adenine at a position corresponding to position 1,397 according to SEQ ID NO:17, and a second primer derived from the 3′ flanking sequence adjacent to an adenine at a position corresponding to position 1,397 according to SEQ ID NO:9, or an adenine at a position corresponding to position 1,397 according to SEQ ID NO:17 to produce an amplicon that is indicative of the presence of the SNP at positions encoding an adenine at a position corresponding to position 1,397 according to SEQ ID NO:9, or an adenine at a position corresponding to position 1,397 according to SEQ ID NO:17. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising an adenine at a position corresponding to position 1,397 according to SEQ ID NO:9, or an adenine at a position corresponding to position 1,397 according to SEQ ID NO:17, and at least 1, 2, 3, 4, 5, 6, 7, 8, 9, 10, or more nucleotides on each side of positions comprising an adenine at a position corresponding to position 1,397 according to SEQ ID NO:9, or an adenine at a position corresponding to position 1,397 according to SEQ ID NO:17.

In some embodiments, to determine whether an ADCY7 nucleic acid molecule (genomic nucleic acid molecule, mRNA molecule, or cDNA molecule), or complement thereof, within a biological sample comprises a nucleotide sequence comprising an adenine at a position corresponding to position 1,344 according to SEQ ID NO:10 (mRNA molecule), or an adenine at a position corresponding to position 1,344 according to SEQ ID NO:18 (cDNA molecule), the biological sample can be subjected to an amplification method using a primer pair that includes a first primer derived from the 5′ flanking sequence adjacent to an adenine at a position corresponding to position 1,344 according to SEQ ID NO:10, or an adenine at a position corresponding to position 1,344 according to SEQ ID NO:18, and a second primer derived from the 3′ flanking sequence adjacent to an adenine at a position corresponding to position 1,344 according to SEQ ID NO:10, or an adenine at a position corresponding to position 1,344 according to SEQ ID NO:18 to produce an amplicon that is indicative of the presence of the SNP at positions encoding an adenine at a position corresponding to position 1,344 according to SEQ ID NO:10, or an adenine at a position corresponding to position 1,344 according to SEQ ID NO:18. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising an adenine at a position corresponding to position 1,344 according to SEQ ID NO:10, or an adenine at a position corresponding to position 1,344 according to SEQ ID NO:18, and at least 1, 2, 3, 4, 5, 6, 7, 8, 9, 10, or more nucleotides on each side of positions comprising an adenine at a position corresponding to position 1,344 according to SEQ ID NO:10, or an adenine at a position corresponding to position 1,344 according to SEQ ID NO:18.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned, and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence-based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of a human ADCY7 predicted loss-of-function polypeptide comprising performing an assay on a sample obtained from a subject to determine whether an ADCY7 polypeptide in the subject contains one or more variations that causes the polypeptide to have a loss-of-function (partial or complete) or predicted loss-of-function (partial or complete). The ADCY7 predicted loss-of-function polypeptide can be any of the ADCY7 truncated variant polypeptides described herein. In some embodiments, the methods detect the presence of ADCY7 Asp439Glu.

In some embodiments, the methods comprise performing an assay on a sample obtained from a subject to determine whether an ADCY7 polypeptide in the sample comprises a glutamic acid at a position corresponding to position 439 according to SEQ ID NO:21. In some embodiments, the methods comprise performing an assay on a sample obtained from a subject to determine whether an ADCY7 polypeptide in the sample comprises a glutamic acid at a position corresponding to position 439 according to SEQ ID NO:22.

In some embodiments, the detecting step comprises sequencing at least a portion of the polypeptide that comprises a position corresponding to position 439 according to SEQ ID NO:21 or SEQ ID NO:19. In some embodiments, the detecting step comprises sequencing at least a portion of the polypeptide that comprises a position corresponding to position 439 according to SEQ ID NO:22 or SEQ ID NO:20.

In some embodiments, the detecting step comprises an immunoassay for detecting the presence of a polypeptide that comprises a position corresponding to position 439 according to SEQ ID NO:21 or SEQ ID NO:19. In some embodiments, the detecting step comprises an immunoassay for detecting the presence of a polypeptide that comprises a position corresponding to position 439 according to SEQ ID NO:22 or SEQ ID NO:20.

In some embodiments, when the subject does not have an ADCY7 predicted loss-of-function polypeptide, then the subject does not have an increased risk for developing an interferon mediated disease or any of SLE, multiple sclerosis, altered white blood count, and production of anti-TG and antinuclear antibodies. In some embodiments, when the subject has an ADCY7 predicted loss-of-function polypeptide, then the subject has an increased risk for developing an interferon mediated disease or any of SLE, multiple sclerosis, altered white blood count, and production of anti-TG and antinuclear antibodies.

The present disclosure also provides isolated nucleic acid molecules that hybridize to ADCY7 variant genomic nucleic acid molecules, ADCY7 variant mRNA molecules, and/or ADCY7 variant cDNA molecules (such as any of the genomic variant nucleic acid molecules, mRNA variant molecules, and cDNA variant molecules disclosed herein). In some embodiments, the isolated nucleic acid molecules hybridize to a portion of the ADCY7 nucleic acid molecule that includes a position corresponding to: position 34,648 according to SEQ ID NO:2; position 1,583 according to SEQ ID NO:7; or position 1,583 according to SEQ ID NO:15. In some embodiments, the isolated nucleic acid molecules hybridize to a portion of the ADCY7 nucleic acid molecule that includes a position corresponding to: position 1,582 according to SEQ ID NO:8; or position 1,582 according to SEQ ID NO:16. In some embodiments, the isolated nucleic acid molecules hybridize to a portion of the ADCY7 nucleic acid molecule that includes a position corresponding: position 1,397 according to SEQ ID NO:9; or position 1,397 according to SEQ ID NO:17. In some embodiments, the isolated nucleic acid molecules hybridize to a portion of the ADCY7 nucleic acid molecule that includes a position corresponding to: position 1,344 according to SEQ ID NO:10; or position 1,344 according to SEQ ID NO:18.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to ADCY7 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to ADCY7 variant genomic nucleic acid molecules, ADCY7 variant mRNA molecules, and/or ADCY7 variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human ADCY7 polypeptide, wherein the portion comprises a position corresponding to: position 34,648 according to SEQ ID NO:2, or the complement thereof; position 1,583 according to SEQ ID NO:7, or the complement thereof; or position 1,583 according to SEQ ID NO:15, or the complement thereof. In some embodiments, the alteration-specific probe or alteration-specific primer comprises a nucleotide sequence which is complementary to a portion of a nucleotide sequence comprising positions corresponding to: positions 34,646 to 34,648 according to SEQ ID NO:2, or the complement thereof; positions 1,581 to 1,583 according to SEQ ID NO:7, or the complement thereof; and/or positions 1,581 to 1,583 according to SEQ ID NO:15, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human ADCY7 polypeptide, wherein the portion comprises a position corresponding to: position 1,582 according to SEQ ID NO:8, or the complement thereof; or position 1,582 according to SEQ ID NO:16, or the complement thereof. In some embodiments, the alteration-specific probe or alteration-specific primer comprises a nucleotide sequence which is complementary to a portion of a nucleotide sequence comprising positions corresponding to: positions 1,580 to 1,582 according to SEQ ID NO:8, or the complement thereof; and/or positions 1,580 to 1,582 according to SEQ ID NO:16, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human ADCY7 polypeptide, wherein the portion comprises a position corresponding to: position 1,397 according to SEQ ID NO:9, or the complement thereof; or position 1,397 according to SEQ ID NO:17, or the complement thereof. In some embodiments, the alteration-specific probe or alteration-specific primer comprises a nucleotide sequence which is complementary to a portion of a nucleotide sequence comprising positions corresponding to: positions 1,395 to 1,397 according to SEQ ID NO:9, or the complement thereof; and/or positions 1,395 to 1,397 according to SEQ ID NO:17, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human ADCY7 polypeptide, wherein the portion comprises a position corresponding to: position 1,344 according to SEQ ID NO:10, or the complement thereof; or position 1,344 according to SEQ ID NO:18, or the complement thereof. In some embodiments, the alteration-specific probe or alteration-specific primer comprises a nucleotide sequence which is complementary to a portion of a nucleotide sequence comprising positions corresponding to: positions 1,342 to 1,344 according to SEQ ID NO:10, or the complement thereof; and/or positions 1,342 to 1,344 according to SEQ ID NO:18, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the ADCY7 variant genomic nucleic acid molecules, ADCY7 variant mRNA molecules, and/or ADCY7 variant cDNA molecules disclosed herein. The primers described herein can be used to amplify ADCY7 variant genomic nucleic acid molecules, ADCY7 variant mRNA molecules, or ADCY7 variant cDNA molecules, or a fragment thereof.

The present disclosure also provides pairs of primers comprising any of the primers described above. For example, if one of the primers' 3′-ends hybridizes to a cytosine at a position corresponding to position 34,648 according to SEQ ID NO:1 (rather than adenine) in a particular ADCY7 nucleic acid molecule, then the presence of the amplified fragment would indicate the presence of an ADCY7 reference genomic nucleic acid molecule. Conversely, if one of the primers' 3′-ends hybridizes to an adenine at a position corresponding to position 34,648 according to SEQ ID NO:2 (rather than cytosine) in a particular ADCY7 nucleic acid molecule, then the presence of the amplified fragment would indicate the presence of the ADCY7 variant genomic nucleic acid molecule. In some embodiments, the nucleotide of the primer complementary to the adenine at a position corresponding to position 34,648 according to SEQ ID NO:2 can be at the 3′ end of the primer. In addition, if one of the primers' 3′-ends hybridizes to a cytosine at a position corresponding to position 1,583 according to SEQ ID NO:3 (rather than adenine) in a particular ADCY7 nucleic acid molecule, then the presence of the amplified fragment would indicate the presence of an ADCY7 reference mRNA molecule. Conversely, if one of the primers' 3′-ends hybridizes to an adenine at a position corresponding to position 1,583 according to SEQ ID NO:7 (rather than cytosine) in a particular ADCY7 mRNA molecule, then the presence of the amplified fragment would indicate the presence of the ADCY7 variant mRNA molecule. In some embodiments, the nucleotide of the primer complementary to the adenine at a position corresponding to position 1,583 according to SEQ ID NO:7 can be at the 3′ end of the primer. In addition, if one of the primers' 3′-ends hybridizes to a cytosine at a position corresponding to position 1,583 according to SEQ ID NO:11 (rather than adenine) in a particular ADCY7 nucleic acid molecule, then the presence of the amplified fragment would indicate the presence of an ADCY7 reference cDNA molecule. Conversely, if one of the primers' 3′-ends hybridizes to an adenine at a position corresponding to position 1,583 according to SEQ ID NO:15 (rather than cytosine) in a particular ADCY7 cDNA molecule, then the presence of the amplified fragment would indicate the presence of the ADCY7 variant cDNA molecule. In some embodiments, the nucleotide of the primer complementary to the adenine at a position corresponding to position 1,583 according to SEQ ID NO:15 can be at the 3′ end of the primer.

If, for example, one of the primers' 3′-ends hybridizes to a cytosine at a position corresponding to position 1,582 according to SEQ ID NO:4 (rather than adenine) in a particular ADCY7 nucleic acid molecule, then the presence of the amplified fragment would indicate the presence of an ADCY7 reference mRNA molecule. Conversely, if one of the primers' 3′-ends hybridizes to an adenine at a position corresponding to position 1,582 according to SEQ ID NO:8 (rather than cytosine) in a particular ADCY7 mRNA molecule, then the presence of the amplified fragment would indicate the presence of the ADCY7 variant mRNA molecule. In some embodiments, the nucleotide of the primer complementary to the adenine at a position corresponding to position 1,582 according to SEQ ID NO:8 can be at the 3′ end of the primer. In addition, if one of the primers' 3′-ends hybridizes to a cytosine at a position corresponding to position 1,582 according to SEQ ID NO:12 (rather than adenine) in a particular ADCY7 nucleic acid molecule, then the presence of the amplified fragment would indicate the presence of an ADCY7 reference cDNA molecule. Conversely, if one of the primers' 3′-ends hybridizes to an adenine at a position corresponding to position 1,582 according to SEQ ID NO:16 (rather than cytosine) in a particular ADCY7 cDNA molecule, then the presence of the amplified fragment would indicate the presence of the ADCY7 variant cDNA molecule. In some embodiments, the nucleotide of the primer complementary to the adenine at a position corresponding to position 1,582 according to SEQ ID NO:16 can be at the 3′ end of the primer.

If, for example, one of the primers' 3′-ends hybridizes to a cytosine at a position corresponding to position 1,397 according to SEQ ID NO:5 (rather than adenine) in a particular ADCY7 nucleic acid molecule, then the presence of the amplified fragment would indicate the presence of an ADCY7 reference mRNA molecule. Conversely, if one of the primers' 3′-ends hybridizes to an adenine at a position corresponding to position 1,397 according to SEQ ID NO:9 (rather than cytosine) in a particular ADCY7 mRNA molecule, then the presence of the amplified fragment would indicate the presence of the ADCY7 variant mRNA molecule. In some embodiments, the nucleotide of the primer complementary to the adenine at a position corresponding to position 1,397 according to SEQ ID NO:9 can be at the 3′ end of the primer. In addition, if one of the primers' 3′-ends hybridizes to a cytosine at a position corresponding to position 1,397 according to SEQ ID NO:13 (rather than adenine) in a particular ADCY7 nucleic acid molecule, then the presence of the amplified fragment would indicate the presence of an ADCY7 reference cDNA molecule. Conversely, if one of the primers' 3′-ends hybridizes to an adenine at a position corresponding to position 1,397 according to SEQ ID NO:17 (rather than cytosine) in a particular ADCY7 cDNA molecule, then the presence of the amplified fragment would indicate the presence of the ADCY7 variant cDNA molecule. In some embodiments, the nucleotide of the primer complementary to the adenine at a position corresponding to position 1,397 according to SEQ ID NO:17 can be at the 3′ end of the primer.

If, for example, one of the primers' 3′-ends hybridizes to cytosine at a position corresponding to position 1,344 according to SEQ ID NO:6 (rather than adenine) in a particular ADCY7 nucleic acid molecule, then the presence of the amplified fragment would indicate the presence of an ADCY7 reference mRNA molecule. Conversely, if one of the primers' 3′-ends hybridizes to an adenine at a position corresponding to position 1,344 according to SEQ ID NO:10 (rather than cytosine) in a particular ADCY7 mRNA molecule, then the presence of the amplified fragment would indicate the presence of the ADCY7 variant mRNA molecule. In some embodiments, the nucleotide of the primer complementary to the adenine at a position corresponding to position 1,344 according to SEQ ID NO:10 can be at the 3′ end of the primer. In addition, if one of the primers' 3′-ends hybridizes to a cytosine at a position corresponding to position 1,344 according to SEQ ID NO:14 (rather than adenine) in a particular ADCY7 nucleic acid molecule, then the presence of the amplified fragment would indicate the presence of an ADCY7 reference cDNA molecule. Conversely, if one of the primers' 3′-ends hybridizes to an adenine at a position corresponding to position 1,344 according to SEQ ID NO:18 (rather than cytosine) in a particular ADCY7 cDNA molecule, then the presence of the amplified fragment would indicate the presence of the ADCY7 variant cDNA molecule. In some embodiments, the nucleotide of the primer complementary to the adenine at a position corresponding to position 1,344 according to SEQ ID NO:18 can be at the 3′ end of the primer.

In the context of the disclosure “specifically hybridizes” means that the probe or primer (such as, for example, the alteration-specific probe or alteration-specific primer) does not hybridize to a nucleic acid sequence encoding an ADCY7 reference genomic nucleic acid molecule, an ADCY7 reference mRNA molecule, and/or an ADCY7 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The present disclosure also provides molecular complexes comprising or consisting of any of the ADCY7 nucleic acid molecules (genomic nucleic acid molecules, mRNA molecules, or cDNA molecules), or complement thereof, described herein and any of the alteration-specific primers or alteration-specific probes described herein. In some embodiments, the ADCY7 nucleic acid molecules (genomic nucleic acid molecules, mRNA molecules, or cDNA molecules), or complement thereof, in the molecular complexes are single-stranded. In some embodiments, the ADCY7 nucleic acid molecule is any of the genomic nucleic acid molecules described herein. In some embodiments, the ADCY7 nucleic acid molecule is any of the mRNA molecules described herein. In some embodiments, the ADCY7 nucleic acid molecule is any of the cDNA molecules described herein. In some embodiments, the molecular complex comprises or consists of any of the ADCY7 nucleic acid molecules (genomic nucleic acid molecules, mRNA molecules, or cDNA molecules), or complement thereof, described herein and any of the alteration-specific primers described herein. In some embodiments, the molecular complex comprises or consists of any of the ADCY7 nucleic acid molecules (genomic nucleic acid molecules, mRNA molecules, or cDNA molecules), or complement thereof, described herein and any of the alteration-specific probes described herein.

In some embodiments, the molecular complex comprises or consists of an alteration-specific primer or an alteration-specific probe hybridized to a genomic nucleic acid molecule comprising a nucleotide sequence encoding a human ADCY7 polypeptide, wherein the alteration-specific primer or the alteration-specific probe is hybridized to an adenine at a position corresponding to position 34,648 according to SEQ ID NO:2, or the complement thereof.

In some embodiments, the molecular complex comprises or consists of an alteration-specific primer or an alteration-specific probe that is hybridized to a GAA codon at positions corresponding to positions 34,646 to 34,648 according to SEQ ID NO:2.

In some embodiments, the molecular complex comprises or consists of a genomic nucleic acid molecule that comprises SEQ ID NO:2.

In some embodiments, the molecular complex comprises or consists of an alteration-specific primer or an alteration-specific probe hybridized to an mRNA molecule comprising a nucleotide sequence encoding a human ADCY7 polypeptide, wherein the alteration-specific primer or the alteration-specific probe is hybridized to: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof.

In some embodiments, the molecular complex comprises or consists of an alteration-specific primer or an alteration-specific probe that is hybridized to: a GAA codon at positions corresponding to positions 1,581 to 1,583 according to SEQ ID NO:7; a GAA codon at positions corresponding to positions 1,580 to 1,582 according to SEQ ID NO:8; a GAA codon at positions corresponding to positions 1,395 to 1,397 according to SEQ ID NO:9; or a GAA codon at positions corresponding to positions 1,342 to 1,344 according to SEQ ID NO:10.

In some embodiments, the molecular complex comprises or consists of an mRNA molecule that comprises SEQ ID NO:7, SEQ ID NO:8, SEQ ID NO:9, or SEQ ID NO:10.

In some embodiments, the molecular complex comprises or consists of an alteration-specific primer or an alteration-specific probe hybridized to a cDNA molecule comprising a nucleotide sequence encoding a human ADCY7 polypeptide, wherein the alteration-specific primer or the alteration-specific probe is hybridized to: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof.

In some embodiments, the molecular complex comprises or consists of an alteration-specific primer or an alteration-specific probe that is hybridized to: a GAA codon at positions corresponding to positions 1,581 to 1,583 according to SEQ ID NO:15; a GAA codon at positions corresponding to positions 1,580 to 1,582 according to SEQ ID NO:16; a GAA codon at positions corresponding to positions 1,395 to 1,397 according to SEQ ID NO:17; or a GAA codon at positions corresponding to positions 1,342 to 1,344 according to SEQ ID NO:18.

In some embodiments, the molecular complex comprises or consists of a cDNA molecule that comprises SEQ ID NO:15, SEQ ID NO:16, SEQ ID NO:17, or SEQ ID NO:18.

In some embodiments, the molecular complex comprises an alteration-specific probe or an alteration-specific primer comprising a label. In some embodiments, the label is a fluorescent label, a radiolabel, or biotin. In some embodiments, the molecular complex further comprises a non-human polymerase.

The nucleotide sequence of an ADCY7 reference genomic nucleic acid molecule is set forth in SEQ ID NO:1. Referring to SEQ ID NO:1, position 34,648 is a cytosine. Referring to SEQ ID NO:1, the nucleotide sequence comprises a GAC codon at positions corresponding to positions 34,646 to 34,648.

A variant genomic nucleic acid molecule of ADCY7 exists, wherein the cytosine at position 34,648 is replaced with an adenine. The nucleotide sequence of this ADCY7 variant genomic nucleic acid molecule is set forth in SEQ ID NO:2.

The nucleotide sequence of an ADCY7 reference mRNA molecule is set forth in SEQ ID NO:3. Referring to SEQ ID NO:3, position 1,583 is a cytosine. Referring to SEQ ID NO:3, the nucleotide sequence comprises a GAC codon at positions corresponding to positions 1,581 to 1,583.

The nucleotide sequence of another ADCY7 reference mRNA molecule is set forth in SEQ ID NO:4. Referring to SEQ ID NO:4, position 1,582 is a cytosine. Referring to SEQ ID NO:4, the nucleotide sequence comprises a GAC codon at positions corresponding to positions 1,580 to 1,582.

The nucleotide sequence of another ADCY7 reference mRNA molecule is set forth in SEQ ID NO:5. Referring to SEQ ID NO:5, position 1,397 is a cytosine. Referring to SEQ ID NO:5, the nucleotide sequence comprises a GAC codon at positions corresponding to positions 1,395 to 1,397.

The nucleotide sequence of another ADCY7 reference mRNA molecule is set forth in SEQ ID NO:6. Referring to SEQ ID NO:6, position 1,344 is a cytosine. Referring to SEQ ID NO:6, the nucleotide sequence comprises a GAC codon at positions corresponding to positions 1,342 to 1,344.

A variant mRNA molecule of ADCY7 exists, wherein the cytosine at position 1,583 (corresponding to position 1,583 according to SEQ ID NO:3) is replaced with an adenine. The nucleotide sequence of this ADCY7 variant mRNA molecule is set forth in SEQ ID NO:7. Referring to SEQ ID NO:7, the nucleotide sequence comprises a GAA codon at positions corresponding to positions 1,581 to 1,583.

The nucleotide sequence of another ADCY7 variant mRNA molecule is set forth in SEQ ID NO:8 (corresponding to the ADCY7 reference mRNA molecule according to SEQ ID NO:4). Referring to SEQ ID NO:8, position 1,582 is an adenine. Referring to SEQ ID NO:8, the nucleotide sequence comprises a GAA codon at positions corresponding to positions 1,580 to 1,582.

The nucleotide sequence of another ADCY7 variant mRNA molecule is set forth in SEQ ID NO:9 (corresponding to the ADCY7 reference mRNA molecule according to SEQ ID NO:5). Referring to SEQ ID NO:9, position 1,397 is an adenine. Referring to SEQ ID NO:9, the nucleotide sequence comprises a GAA codon at positions corresponding to positions 1,395 to 1,397.

The nucleotide sequence of another ADCY7 variant mRNA molecule is set forth in SEQ ID NO:10 (corresponding to the ADCY7 reference mRNA molecule according to SEQ ID NO:6). Referring to SEQ ID NO:10, position 1,344 is an adenine. Referring to SEQ ID NO:10, the nucleotide sequence comprises a GAA codon at positions corresponding to positions 1,342 to 1,344.

The nucleotide sequence of an ADCY7 reference cDNA molecule is set forth in SEQ ID NO:11. Referring to SEQ ID NO:11, position 1,583 is a cytosine. Referring to SEQ ID NO:11, the nucleotide sequence comprises a GAC codon at positions corresponding to positions 1,581 to 1,583.

The nucleotide sequence of another ADCY7 reference cDNA molecule is set forth in SEQ ID NO:12. Referring to SEQ ID NO:12, position 1,582 is a cytosine. Referring to SEQ ID NO:12, the nucleotide sequence comprises a GAC codon at positions corresponding to positions 1,580 to 1,582.

The nucleotide sequence of another ADCY7 reference cDNA molecule is set forth in SEQ ID NO:13. Referring to SEQ ID NO:13, position 1,397 is a cytosine. Referring to SEQ ID NO:13, the nucleotide sequence comprises a GAC codon at positions corresponding to positions 1,395 to 1,397.

The nucleotide sequence of another ADCY7 reference cDNA molecule is set forth in SEQ ID NO:14. Referring to SEQ ID NO:14, position 1,344 is a cytosine. Referring to SEQ ID NO:14, the nucleotide sequence comprises a GAC codon at positions corresponding to positions 1,342 to 1,344.

A variant cDNA molecule of ADCY7 exists, wherein the cytosine at position 1,583 (corresponding to position 1,583 according to SEQ ID NO:11) is replaced with an adenine. The nucleotide sequence of this ADCY7 variant cDNA molecule is set forth in SEQ ID NO:15. Referring to SEQ ID NO:15, the nucleotide sequence comprises a GAA codon at positions corresponding to positions 1,581 to 1,583.

The nucleotide sequence of another ADCY7 variant cDNA molecule is set forth in SEQ ID NO:16 (corresponding to the ADCY7 reference cDNA molecule according to SEQ ID NO:12). Referring to SEQ ID NO:16, position 1,582 is an adenine. Referring to SEQ ID NO:16, the nucleotide sequence comprises a GAA codon at positions corresponding to positions 1,580 to 1,582.

The nucleotide sequence of another ADCY7 variant cDNA molecule is set forth in SEQ ID NO:17 (corresponding to the ADCY7 reference cDNA molecule according to SEQ ID NO:13). Referring to SEQ ID NO:17, position 1,397 is an adenine. Referring to SEQ ID NO:17, the nucleotide sequence comprises a GAA codon at positions corresponding to positions 1,395 to 1,397.

The nucleotide sequence of another ADCY7 variant cDNA molecule is set forth in SEQ ID NO:18 (corresponding to the ADCY7 reference cDNA molecule according to SEQ ID NO:14). Referring to SEQ ID NO:18, position 1,344 is an adenine. Referring to SEQ ID NO:18, the nucleotide sequence comprises a GAA codon at positions corresponding to positions 1,342 to 1,344.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (1), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NOs:3-6, or SEQ ID NOs:11-14). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, a nucleic acid molecule comprising a nucleotide sequence encoding a human ADCY7 polypeptide, wherein the nucleotide sequence comprises an adenine at a position corresponding to position 34,648 according to SEQ ID NO:2 means that if the nucleotide sequence of the ADCY7 genomic nucleic acid molecule is aligned to the sequence of SEQ ID NO:2, the ADCY7 sequence has an adenine residue at the position that corresponds to position 34,648 of SEQ ID NO:2. The same applies for mRNA molecules comprising a nucleotide sequence encoding a human ADCY7 polypeptide, wherein the nucleotide sequence comprises an adenine at a position corresponding to position 1,583 according to SEQ ID NO:7, and cDNA molecules comprising a nucleotide sequence encoding a human ADCY7 polypeptide, wherein the nucleotide sequence comprises an adenine at a position corresponding to position 1,583 according to SEQ ID NO:15. In other words, these phrases refer to a nucleic acid molecule encoding an ADCY7 polypeptide, wherein the genomic nucleic acid molecule has a nucleotide sequence that comprises an adenine residue that is homologous to the adenine residue at position 34,648 of SEQ ID NO:2 (or wherein the mRNA molecule has a nucleotide sequence that comprises an adenine residue that is homologous to the adenine residue at position 1,583 of SEQ ID NO:7, or wherein the cDNA molecule has a nucleotide sequence that comprises an adenine residue that is homologous to the adenine residue at position 1,583 of SEQ ID NO:15). Herein, such a sequence is also referred to as “ADCY7 sequence with the Asp439Glu alteration” or “ADCY7 sequence with the Asp439Glu variation” referring to genomic nucleic acid molecules (or “ADCY7 sequence with the C1,583A alteration” or “ADCY7 sequence with the C1,583A variation” referring to mRNA molecules, and “ADCY7 sequence with the C1,583A alteration” or “ADCY7 sequence with the C1,583A variation” referring to cDNA molecules).

As described herein, a position within an ADCY7 genomic nucleic acid molecule that corresponds to position 34,648 according to SEQ ID NO:2, for example, can be identified by performing a sequence alignment between the nucleotide sequence of a particular ADCY7 nucleic acid molecule and the nucleotide sequence of SEQ ID NO:2. A variety of computational algorithms exist that can be used for performing a sequence alignment to identify a nucleotide position that corresponds to, for example, position 34,648 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequence of an ADCY7 reference polypeptide is set forth in SEQ ID NO:19. Referring to SEQ ID NO:19, the ADCY7 reference polypeptide is 734 amino acids in length. Referring to SEQ ID NO:19, position 439 is an aspartic acid.

The amino acid sequence of another ADCY7 reference polypeptide is set forth in SEQ ID NO:20. Referring to SEQ ID NO:20, the ADCY7 reference polypeptide is 1,080 amino acids in length. Referring to SEQ ID NO:20, position 439 is an aspartic acid.

An ADCY7 variant polypeptide exists (Asp439Glu or D439E), the amino acid sequence of which is set forth in SEQ ID NO:21. Referring to SEQ ID NO:21, the ADCY7 variant polypeptide is 734 amino acids in length. Referring to SEQ ID NO:21, position 439 is a glutamic acid.

Another ADCY7 variant polypeptide exists (Asp439Glu or D439E), the amino acid sequence of which is set forth in SEQ ID NO:22. Referring to SEQ ID NO:22, the ADCY7 variant polypeptide is 1,080 amino acids in length. Referring to SEQ ID NO:22, position 439 is a glutamic acid.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or inhibit an interferon mediated disease for use in the treatment of an interferon mediated disease (or for use in the preparation of a medicament for treating an interferon mediated disease) in a subject, wherein the subject has any of the variant genomic nucleic acid molecules, mRNA molecules, and/or cDNA molecules encoding a human ADCY7 variant polypeptide described herein. The therapeutic agents that treat or inhibit an interferon mediated disease can be any of the therapeutic agents that treat or inhibit an interferon mediated disease described herein.

In some embodiments, the subject comprises: a genomic nucleic acid molecule having a nucleotide sequence encoding a human ADCY7 polypeptide, wherein the nucleotide sequence comprises an adenine at a position corresponding to position 34,648 according to SEQ ID NO:2, or the complement thereof; an mRNA molecule having a nucleotide sequence encoding a human ADCY7 polypeptide, wherein the nucleotide sequence comprises an adenine at a position corresponding to position 1,583 according to SEQ ID NO:7, or the complement thereof; a cDNA molecule having a nucleotide sequence encoding a human ADCY7 polypeptide, wherein the nucleotide sequence comprises an adenine at a position corresponding to position 1,583 according to SEQ ID NO:15, or the complement thereof; or an ADCY7 polypeptide that comprises a glutamic acid at a position corresponding to position 439 according to SEQ ID NO:21.

In some embodiments, the subject comprises: an mRNA molecule having a nucleotide sequence encoding a human ADCY7 polypeptide, wherein the nucleotide sequence comprises an adenine at a position corresponding to position 1,582 according to SEQ ID NO:8, or the complement thereof; a cDNA molecule having a nucleotide sequence encoding a human ADCY7 polypeptide, wherein the nucleotide sequence comprises an adenine at a position corresponding to position 1,582 according to SEQ ID NO:16, or the complement thereof; or an ADCY7 polypeptide that comprises a glutamic acid at a position corresponding to position 439 according to SEQ ID NO:22.

In some embodiments, the subject comprises: an mRNA molecule having a nucleotide sequence encoding a human ADCY7 polypeptide, wherein the nucleotide sequence comprises an adenine at a position corresponding to position 1,397 according to SEQ ID NO:9, or the complement thereof; a cDNA molecule having a nucleotide sequence encoding a human ADCY7 polypeptide, wherein the nucleotide sequence comprises an adenine at a position corresponding to position 1,397 according to SEQ ID NO:17, or the complement thereof; or an ADCY7 polypeptide that comprises a glutamic acid at a position corresponding to position 439 according to SEQ ID NO:22.

In some embodiments, the subject comprises: an mRNA molecule having a nucleotide sequence encoding a human ADCY7 polypeptide, wherein the nucleotide sequence comprises an adenine at a position corresponding to position 1,344 according to SEQ ID NO:10, or the complement thereof; a cDNA molecule having a nucleotide sequence encoding a human ADCY7 polypeptide, wherein the nucleotide sequence comprises an adenine at a position corresponding to position 1,344 according to SEQ ID NO:18, or the complement thereof; or an ADCY7 polypeptide that comprises a glutamic acid at a position corresponding to position 439 according to SEQ ID NO:22.

The following representative embodiments are presented:

Embodiment 1. A method of identifying a subject having an increased risk for developing an interferon mediated disease, wherein the method comprises: determining or having determined the presence or absence of an Adenylate Cyclase 7 (ADCY7) predicted loss-of-function variant nucleic acid molecule encoding a human ADCY7 polypeptide in a biological sample obtained from the subject; wherein: when the subject is ADCY7 reference, then the subject does not have an increased risk for developing an interferon mediated disease; and when the subject is heterozygous for an ADCY7 predicted loss-of-function variant or homozygous for an ADCY7 predicted loss-of-function variant, then the subject has an increased risk for developing an interferon mediated disease.

Embodiment 2. The method according to embodiment 1, wherein the ADCY7 predicted loss-of-function variant nucleic acid molecule is a nucleic acid molecule encoding ADCY7 Asp439Glu.

Embodiment 3. The method according to embodiment 2, wherein the ADCY7 predicted loss-of-function variant nucleic acid molecule is: a genomic nucleic acid molecule having a nucleotide sequence comprising an adenine at a position corresponding to position 34,648 according to SEQ ID NO:2; an mRNA molecule having a nucleotide sequence comprising: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10; or a cDNA molecule produced from an mRNA molecule, wherein the cDNA molecule has a nucleotide sequence comprising: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18.

Embodiment 4. The method according to any one of embodiments 1 to 3, wherein the determining step is carried out in vitro.

Embodiment 5. The method according to any one of embodiments 1 to 4, wherein the determining step comprises sequencing at least a portion of the nucleotide sequence of the ADCY7 genomic nucleic acid molecule in the biological sample, wherein the sequenced portion comprises a position corresponding to position 34,648 according to SEQ ID NO:2, or the complement thereof; wherein when the sequenced portion of the ADCY7 genomic nucleic acid molecule in the biological sample comprises an adenine at a position corresponding to position 34,648 according to SEQ ID NO:2, then the ADCY7 genomic nucleic acid molecule in the biological sample is an ADCY7 predicted loss-of-function variant genomic nucleic acid molecule.

Embodiment 6. The method according to any one of embodiments 1 to 4, wherein the determining step comprises sequencing at least a portion of the nucleotide sequence of the ADCY7 mRNA molecule in the biological sample, wherein the sequenced portion comprises a position corresponding to: position 1,583 according to SEQ ID NO:7, or the complement thereof; position 1,582 according to SEQ ID NO:8, or the complement thereof; position 1,397 according to SEQ ID NO:9, or the complement thereof; or position 1,344 according to SEQ ID NO:10, or the complement thereof; wherein when the sequenced portion of the ADCY7 mRNA molecule in the biological sample comprises: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10; then the ADCY7 mRNA molecule in the biological sample is an ADCY7 predicted loss-of-function variant mRNA molecule.

Embodiment 7. The method according to any one of embodiments 1 to 4, wherein the determining step comprises sequencing at least a portion of the nucleotide sequence of the ADCY7 cDNA molecule in the biological sample, wherein the sequenced portion comprises a position corresponding to: position 1,583 according to SEQ ID NO:15, or the complement thereof; position 1,582 according to SEQ ID NO:16, or the complement thereof; position 1,397 according to SEQ ID NO:17, or the complement thereof; or position 1,344 according to SEQ ID NO:18, or the complement thereof; wherein when the sequenced portion of the ADCY7 cDNA molecule in the biological sample comprises: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18; then the ADCY7 cDNA molecule in the biological sample is an ADCY7 predicted loss-of-function variant cDNA molecule.

Embodiment 8. The method according to any one of embodiments 1 to 4, wherein the determining step comprises: a) contacting the biological sample with a primer hybridizing to a portion of the nucleotide sequence of the ADCY7 genomic nucleic acid molecule that is proximate to a position corresponding to position 34,648 according to SEQ ID NO:2; b) extending the primer at least through the position of the nucleotide sequence of the ADCY7 genomic nucleic acid molecule corresponding to position 34,648 according to SEQ ID NO:2; and c) determining whether the extension product of the primer comprises an adenine at a position corresponding to position 34,648 according to SEQ ID NO:2.

Embodiment 9. The method according to any one of embodiments 1 to 4, wherein the determining step comprises: a) contacting the biological sample with a primer hybridizing to a portion of the nucleotide sequence of the ADCY7 mRNA molecule that is proximate to a position corresponding to: position 1,583 according to SEQ ID NO:7; position 1,582 according to SEQ ID NO:8; position 1,397 according to SEQ ID NO:9; or position 1,344 according to SEQ ID NO:10; b) extending the primer at least through the position of the nucleotide sequence of the ADCY7 mRNA molecule corresponding to: position 1,583 according to SEQ ID NO:7; position 1,582 according to SEQ ID NO:8; position 1,397 according to SEQ ID NO:9; or position 1,344 according to SEQ ID NO:10; and c) determining whether the extension product of the primer comprises: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10.

Embodiment 10. The method according to any one of embodiments 1 to 4, wherein the determining step comprises: a) contacting the biological sample with a primer hybridizing to a portion of the nucleotide sequence of the ADCY7 cDNA molecule that is proximate to a position corresponding to: position 1,583 according to SEQ ID NO:15; position 1,582 according to SEQ ID NO:16; position 1,397 according to SEQ ID NO:17; or position 1,344 according to SEQ ID NO:18; b) extending the primer at least through the position of the nucleotide sequence of the ADCY7 cDNA molecule corresponding to: position 1,583 according to SEQ ID NO:15; position 1,582 according to SEQ ID NO:16; position 1,397 according to SEQ ID NO:17; or position 1,344 according to SEQ ID NO:18; and c) determining whether the extension product of the primer comprises: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18.

Embodiment 11. The method according to any one of embodiments 5 to 10, wherein the determining step comprises sequencing the entire nucleic acid molecule.

Embodiment 12. The method according to any one of embodiments 1 to 4, wherein the determining step comprises: a) amplifying at least a portion of the nucleic acid molecule that encodes the human ADCY7 polypeptide, wherein the portion comprises an adenine at a position corresponding to position 34,648 according to SEQ ID NO:2, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising an adenine at a position corresponding to position 34,648 according to SEQ ID NO:2, or the complement thereof; and d) detecting the detectable label.

Embodiment 13. The method according to any one of embodiments 1 to 4, wherein the determining step comprises: a) amplifying at least a portion of the nucleic acid molecule that encodes the human ADCY7 polypeptide, wherein the portion comprises: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; and d) detecting the detectable label.

Embodiment 14. The method according to any one of embodiments 1 to 4, wherein the determining step comprises: a) amplifying at least a portion of the nucleic acid molecule that encodes the human ADCY7 polypeptide, wherein the portion comprises: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; and d) detecting the detectable label.

Embodiment 15. The method according to embodiment 14, wherein the nucleic acid molecule in the sample is mRNA and the mRNA is reverse-transcribed into cDNA prior to the amplifying step.

Embodiment 16. The method according to any one of embodiments 1 to 4, wherein the detecting step comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising an adenine at a position corresponding to position 34,648 according to SEQ ID NO:2, or the complement thereof; and detecting the detectable label.

Embodiment 17. The method according to any one of embodiments 1 to 4, wherein the detecting step comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; and detecting the detectable label.

Embodiment 18. The method according to any one of embodiments 1 to 4, wherein the detecting step comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; and detecting the detectable label.

Embodiment 19. The method according to any one of embodiments 1 to 18, wherein the subject is heterozygous or homozygous for an ADCY7 predicted loss-of-function variant, and the subject is further administered a therapeutic agent that treats or inhibits the interferon mediated disease.

Embodiment 20. The method according to any one of embodiments 1 to 19, wherein the interferon mediated disease is multiple sclerosis.

Embodiment 21. A method of treating a subject with a therapeutic agent that treats or inhibits an interferon mediated disease, wherein the subject is suffering from an interferon mediated disease, the method comprising the steps of: determining whether the subject has an Adenylate Cyclase 7 (ADCY7) predicted loss-of-function variant nucleic acid molecule encoding a human ADCY7 polypeptide by: obtaining or having obtained a biological sample from the subject; and performing or having performed a genotyping assay on the biological sample to determine if the subject has a genotype comprising the ADCY7 predicted loss-of-function variant nucleic acid molecule; and when the subject is ADCY7 reference, then administering or continuing to administer to the subject the therapeutic agent that treats or inhibits interferon mediated disease in a standard dosage amount; and when the subject is heterozygous or homozygous for an ADCY7 predicted loss-of-function variant, then administering or continuing to administer to the subject the therapeutic agent that treats or inhibits interferon mediated disease in an amount that is the same as or greater than a standard dosage amount; wherein the presence of a genotype having the ADCY7 predicted loss-of-function variant nucleic acid molecule encoding the human ADCY7 polypeptide indicates the subject has an increased risk of developing interferon mediated disease.

Embodiment 22. The method according to embodiment 21, wherein the ADCY7 predicted loss-of-function variant nucleic acid molecule is a nucleic acid molecule encoding ADCY7 Asp439Glu.

Embodiment 23. The method according to embodiment 22, wherein the ADCY7 predicted loss-of-function variant nucleic acid molecule is: a genomic nucleic acid molecule having a nucleotide sequence comprising an adenine at a position corresponding to position 34,648 according to SEQ ID NO:2; an mRNA molecule having a nucleotide sequence comprising: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10; or a cDNA molecule produced from an mRNA molecule, wherein the cDNA molecule has a nucleotide sequence comprising: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18.

Embodiment 24. The method according to any one of embodiments 21 to 23, wherein the genotyping assay comprises sequencing at least a portion of the nucleotide sequence of the ADCY7 genomic nucleic acid molecule in the biological sample, wherein the sequenced portion comprises a position corresponding to position 34,648 according to SEQ ID NO:2, or the complement thereof; wherein when the sequenced portion of the ADCY7 genomic nucleic acid molecule in the biological sample comprises an adenine at a position corresponding to position 34,648 according to SEQ ID NO:2, then the ADCY7 genomic nucleic acid molecule in the biological sample is an ADCY7 predicted loss-of-function variant genomic nucleic acid molecule.

Embodiment 25. The method according to any one of embodiments 21 to 23, wherein the genotyping assay comprises sequencing at least a portion of the nucleotide sequence of the ADCY7 mRNA molecule in the biological sample, wherein the sequenced portion comprises a position corresponding to: position 1,583 according to SEQ ID NO:7, or the complement thereof; position 1,582 according to SEQ ID NO:8, or the complement thereof; position 1,397 according to SEQ ID NO:9, or the complement thereof; or position 1,344 according to SEQ ID NO:10, or the complement thereof; wherein when the sequenced portion of the ADCY7 mRNA molecule in the biological sample comprises: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10; then the ADCY7 mRNA molecule in the biological sample is an ADCY7 predicted loss-of-function variant mRNA molecule.

Embodiment 26. The method according to any one of embodiments 21 to 23, wherein the genotyping assay comprises sequencing at least a portion of the nucleotide sequence of the ADCY7 cDNA molecule in the biological sample, wherein the sequenced portion comprises a position corresponding to: position 1,583 according to SEQ ID NO:15, or the complement thereof; position 1,582 according to SEQ ID NO:16, or the complement thereof; position 1,397 according to SEQ ID NO:17, or the complement thereof; or position 1,344 according to SEQ ID NO:18, or the complement thereof; wherein when the sequenced portion of the ADCY7 cDNA molecule in the biological sample comprises: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18; then the ADCY7 cDNA molecule in the biological sample is an ADCY7 predicted loss-of-function variant cDNA molecule.

Embodiment 27. The method according to any one of embodiments 21 to 23, wherein the genotyping assay comprises: a) contacting the biological sample with a primer hybridizing to a portion of the nucleotide sequence of the ADCY7 genomic nucleic acid molecule that is proximate to a position corresponding to position 34,648 according to SEQ ID NO:2; b) extending the primer at least through the position of the nucleotide sequence of the ADCY7 genomic nucleic acid molecule corresponding to position 34,648 according to SEQ ID NO:2; and c) determining whether the extension product of the primer comprises an adenine at a position corresponding to position 34,648 according to SEQ ID NO:2.

Embodiment 28. The method according to any one of embodiments 21 to 23, wherein the genotyping assay comprises: a) contacting the biological sample with a primer hybridizing to a portion of the nucleotide sequence of the ADCY7 mRNA molecule that is proximate to a position corresponding to: position 1,583 according to SEQ ID NO:7; position 1,582 according to SEQ ID NO:8; position 1,397 according to SEQ ID NO:9; or position 1,344 according to SEQ ID NO:10; b) extending the primer at least through the position of the nucleotide sequence of the ADCY7 mRNA molecule corresponding to: position 1,583 according to SEQ ID NO:7; position 1,582 according to SEQ ID NO:8; position 1,397 according to SEQ ID NO:9; or position 1,344 according to SEQ ID NO:10; and c) determining whether the extension product of the primer comprises: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10.

Embodiment 29. The method according to any one of embodiments 21 to 23, wherein the genotyping assay comprises: a) contacting the biological sample with a primer hybridizing to a portion of the nucleotide sequence of the ADCY7 cDNA molecule that is proximate to a position corresponding to: position 1,583 according to SEQ ID NO:15; position 1,582 according to SEQ ID NO:16; position 1,397 according to SEQ ID NO:17; or position 1,344 according to SEQ ID NO:18; b) extending the primer at least through the position of the nucleotide sequence of the ADCY7 cDNA molecule corresponding to: position 1,583 according to SEQ ID NO:15; position 1,582 according to SEQ ID NO:16; position 1,397 according to SEQ ID NO:17; or position 1,344 according to SEQ ID NO:18; and c) determining whether the extension product of the primer comprises: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18.

Embodiment 30. The method according to any one of embodiments 24 to 29, wherein the genotyping assay comprises sequencing the entire nucleic acid molecule.

Embodiment 31. The method according to any one of embodiments 21 to 23, wherein the genotyping assay comprises: a) amplifying at least a portion of the nucleic acid molecule that encodes the human ADCY7 polypeptide, wherein the portion comprises an adenine at a position corresponding to position 34,648 according to SEQ ID NO:2, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising an adenine at a position corresponding to position 34,648 according to SEQ ID NO:2, or the complement thereof; and d) detecting the detectable label.

Embodiment 32. The method according to any one of embodiments 21 to 23, wherein the genotyping assay comprises: a) amplifying at least a portion of the nucleic acid molecule that encodes the human ADCY7 polypeptide, wherein the portion comprises: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; and d) detecting the detectable label.

Embodiment 33. The method according to any one of embodiments 21 to 23, wherein the genotyping assay comprises: a) amplifying at least a portion of the nucleic acid molecule that encodes the human ADCY7 polypeptide, wherein the portion comprises: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; and d) detecting the detectable label.

Embodiment 34. The method according to embodiment 33, wherein the nucleic acid molecule in the sample is mRNA and the mRNA is reverse-transcribed into cDNA prior to the amplifying step.

Embodiment 35. The method according to any one of embodiments 21 to 23, wherein the genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising an adenine at a position corresponding to position 34,648 according to SEQ ID NO:2, or the complement thereof; and detecting the detectable label.

Embodiment 36. The method according to any one of embodiments 21 to 23, wherein the genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; and detecting the detectable label.

Embodiment 37. The method according to any one of embodiments 21 to 23, wherein the genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; and detecting the detectable label.

Embodiment 38. The method according to any one of embodiments 21 to 37, wherein the nucleic acid molecule is present within a cell obtained from the subject.

Embodiment 39. The method according to any one of embodiments 21 to 38, wherein the interferon mediated disease is multiple sclerosis.

Embodiment 40. A method of detecting a human Adenylate Cyclase 7 (ADCY7) variant nucleic acid molecule in a subject comprising assaying a sample obtained from the subject to determine whether a nucleic acid molecule in the sample is: a genomic nucleic acid molecule comprising a nucleotide sequence comprising: an adenine at a position corresponding to position 34,648 according to SEQ ID NO:2, or the complement thereof; an mRNA molecule comprising a nucleotide sequence comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; or a cDNA molecule comprising a nucleotide sequence comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof.

Embodiment 41. The method according to embodiment 40, wherein the method is an in vitro method.

Embodiment 42. The method according to embodiment 40 or embodiment 41, wherein the assay comprises sequencing at least a portion of the nucleic acid molecule, wherein the sequenced portion comprises an adenine at a position corresponding to position 34,648 according to SEQ ID NO:2, or the complement thereof.

Embodiment 43. The method according to embodiment 40 or embodiment 41, wherein the assay comprises sequencing at least a portion of the nucleic acid molecule, wherein the sequenced portion comprises: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof.

Embodiment 44. The method according to embodiment 40 or embodiment 41, wherein the assay comprises sequencing at least a portion of the nucleic acid molecule, wherein the sequenced portion comprises: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof.

Embodiment 45. The method according to embodiment 40 or embodiment 41, wherein the assay comprises: a) contacting the sample with a primer hybridizing to a portion of the nucleotide sequence of the ADCY7 genomic nucleic acid molecule that is proximate to a position corresponding to position 34,648 according to SEQ ID NO:2; b) extending the primer at least through the position of the nucleotide sequence of the ADCY7 genomic nucleic acid molecule corresponding to position 34,648 according to SEQ ID NO:2; and c) determining whether the extension product of the primer comprises: an adenine at a position corresponding to position 34,648 according to SEQ ID NO:2.

Embodiment 46. The method according to embodiment 40 or embodiment 41, wherein the assay comprises: a) contacting the sample with a primer hybridizing to a portion of the nucleotide sequence of the ADCY7 mRNA molecule that is proximate to a position corresponding to: position 1,583 according to SEQ ID NO:7; position 1,582 according to SEQ ID NO:8; position 1,397 according to SEQ ID NO:9; or position 1,344 according to SEQ ID NO:10; b) extending the primer at least through the position of the nucleotide sequence of the ADCY7 mRNA molecule corresponding to: position 1,583 according to SEQ ID NO:7; position 1,582 according to SEQ ID NO:8; position 1,397 according to SEQ ID NO:9; or position 1,344 according to SEQ ID NO:10; and c) determining whether the extension product of the primer comprises: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10.

Embodiment 47. The method according to embodiment 40 or embodiment 41, wherein the assay comprises: a) contacting the sample with a primer hybridizing to a portion of the nucleotide sequence of the ADCY7 cDNA molecule that is proximate to a position corresponding to: position 1,583 according to SEQ ID NO:15; position 1,582 according to SEQ ID NO:16; position 1,397 according to SEQ ID NO:17; or position 1,344 according to SEQ ID NO:18; b) extending the primer at least through the position of the nucleotide sequence of the ADCY7 cDNA molecule corresponding to: position 1,583 according to SEQ ID NO:15; position 1,582 according to SEQ ID NO:16; position 1,397 according to SEQ ID NO:17; or position 1,344 according to SEQ ID NO:18; and c) determining whether the extension product of the primer comprises: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18.

Embodiment 48. The method according to any one of embodiments 42 to 47, wherein the assay comprises sequencing the entire nucleic acid molecule.

Embodiment 49. The method according to embodiment 40 or embodiment 41, wherein the assay comprises: a) amplifying at least a portion of the nucleic acid molecule that encodes the human ADCY7 polypeptide, wherein the portion comprises an adenine at a position corresponding to position 34,648 according to SEQ ID NO:2, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 34,648 according to SEQ ID NO:2, or the complement thereof; and d) detecting the detectable label.

Embodiment 50. The method according to embodiment 40 or embodiment 41, wherein the assay comprises: a) amplifying at least a portion of the nucleic acid molecule that encodes the human ADCY7 polypeptide, wherein the portion comprises: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; and d) detecting the detectable label.

Embodiment 51. The method according to embodiment 40 or embodiment 41, wherein the assay comprises: a) amplifying at least a portion of the nucleic acid molecule that encodes the human ADCY7 polypeptide, wherein the portion comprises: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; and d) detecting the detectable label.

Embodiment 52. The method according to embodiment 51, wherein the nucleic acid molecule in the sample is mRNA and the mRNA is reverse-transcribed into cDNA prior to the amplifying step.

Embodiment 53. The method according to embodiment 40 or embodiment 41, wherein the assay comprises: contacting the nucleic acid molecule with an alteration-specific probe comprising a detectable label, wherein the alteration-specific probe comprises a nucleotide sequence which hybridizes under stringent conditions to the nucleic acid sequence of the amplified nucleic acid molecule comprising an adenine at a position corresponding to position 34,648 according to SEQ ID NO:2, or the complement thereof; and detecting the detectable label.

Embodiment 54. The method according to embodiment 40 or embodiment 41, wherein the assay comprises: contacting the nucleic acid molecule with an alteration-specific probe comprising a detectable label, wherein the alteration-specific probe comprises a nucleotide sequence which hybridizes under stringent conditions to the nucleic acid sequence of the amplified nucleic acid molecule comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; and detecting the detectable label.

Embodiment 55. The method according to embodiment 40 or embodiment 41, wherein the assay comprises: contacting the nucleic acid molecule with an alteration-specific probe comprising a detectable label, wherein the alteration-specific probe comprises a nucleotide sequence which hybridizes under stringent conditions to the nucleic acid sequence of the amplified nucleic acid molecule comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; and detecting the detectable label.

Embodiment 56. The method according to any one of embodiments 40 to 55, wherein the nucleic acid molecule is present within a cell obtained from the subject.

Embodiment 57. A method of detecting the presence of a human Adenylate Cyclase 7 (ADCY7) Asp439Glu variant polypeptide, comprising performing an assay on a sample obtained from a subject to determine whether an ADCY7 protein in the sample comprises: a glutamic acid at a position corresponding to position 439 according to SEQ ID NO:21; or a glutamic acid at a position corresponding to position 439 according to SEQ ID NO:22.

Embodiment 58. The method according to embodiment 57, wherein the assay comprises sequencing the polypeptide.

Embodiment 59. The method according to embodiment 57, wherein the assay is an immunoassay.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: ADCY7 Asp439Glu Association Analysis

The UK Biobank (UKB) is a prospective population-based study of over 500,000 individuals with extensive and readily accessible phenotypic and genetic data. Publicly available genome-wide array data (UKB 500K Genotyped) were extended with whole exome sequencing for 150,000 individuals (UKB Freeze Five), which allows direct assessment of all protein-altering variants. The subset of European ancestry individuals was used for genetic association analysis. Cases and controls were compared using logistic regression with ancestry and batch as covariates to control for potential confounders. The same statical method was used to analyze whole exome data from the European subset of 90,000 individuals from Geisinger Healthsystem cohort (GHS Freeze 90 Meta). Observed counts of variant reference and alternative alleles (RR:RA:AA) are shown together with estimated effects (Odds ratio), and confidence intervals (CI), and allele frequency (AAF). Table 1 shows association of ADCY7 Asp439Glu with SLE.

TABLE 1

| Pheno- | Effect | P-value | Cases/Controls | | |
|---|---|---|---|---|---|
| type | [Cl] | (FDR) | (RR:RA:AA) | AAF | Cohort |
| 1 | 5.012 | 2.236E−05 | Cases:198 | 0.00595 | UKB |
| | [2.38, 10.6] | (0.711) | (187:11:0) | | Freeze |
| | | | Controls: 141517 | | Five EUR |
| | | | (139848:1664:5) | | |
| 2 | 5.082 | 0.0005078 | Cases: 127 | 0.00594 | UKB |
| | [2.03, 12.7] | (0.638) | (120:7:0) | | Freeze |
| | | | Controls: 141595 | | Five EUR |
| | | | (139922:1668:5) | | |

1 = Systemic lupus erythematosus (SLE), self-reported.
2 = Systemic lupus erythematosus (SLE), ICD-based case definition.

TABLE 2
shows association of ADCY7 Asp439Glu with connective tissue disease.

| | Effect | P-value | Cases/Controls | | |
|---|---|---|---|---|---|
| Phenotype | [Cl] | (FDR) | (RR:RA:AA) | AAF | Cohort |
| 1 | 1.476 | 0.02578 | Cases: 2072 | 0.00580 | GHS |
| | [1.05, | (0.733) | (2039:32:1) | | Freeze |
| | 2.08] | | Controls: 83557 | | 90 Meta |
| | | | (82599:956:2) | | EUR |
| 1 | 1.384 | 0.1354 | Cases: 1291 | 0.00592 | UKB 500K |
| | [0.903, | (0.943) | (1270:21:0) | | Genotyped |
| | 2.12] | | Controls: 459884 | | EUR |
| | | | (454462:5406:16) | | |

1 = Other systemic involvement of connective tissue.

Table 3 shows associations of ADCY7 Asp439Glu with anti-TG and ANA autoantibodies, which are diagnostic markers for the above diseases.

TABLE 3

| | | P-value | Cases/Controls | | |
|---|---|---|---|---|---|
| Phenotype | Effect [Cl] | (FDR) | (RR:RA:AA) | AAF | Cohort |
| 1 | 2.046 | 0.003888 | Cases: 1340 | 0.00656 | GHS |
| | [1.26, 3.33] | (0.639) | (1311:29:0) | | Freeze 90 |
| | | | Controls: 3916 | | Meta |
| | | | (3876:40:0) | | EUR |
| 2 | 1.391 | 0.009523 | Cases: 7061 | 0.00571 | GHS |
| | [1.08, 1.78] | (0.747) | (6965:95:1) | | Freeze 90 |
| | | | Controls: 18339 | | Meta |
| | | | (18147:191:1) | | EUR |

1 = Anti-Thyroglobulin Antibody.
2 = Anti-Nuclear Antibody.

Table 4 shows association of ADCY7 Asp439Glu with multiple sclerosis in UKB.

TABLE 4

| Pheno- | Effect | P-value | Cases/Controls | | |
|---|---|---|---|---|---|
| type | [Cl] | (FDR) | (RR:RA:AA) | AAF | Cohort |
| 1 | 1.775 | 0.003366 | Cases: 1266 | 0.00592 | UKB |
| | [1.21, | (0.444) | (1240:26:0) | | 500K |
| | 2.61] | | Controls: 461435 | | Genotyped |
| | | | (456000:5419:16) | | EUR |

1 = Multiple sclerosis.

Table 5 shows association of ADCY7 Asp439Glu with multiple sclerosis in GHS.

TABLE 5

| | Effect | P-value | Cases/Controls | | |
|---|---|---|---|---|---|
| Phenotype | [Cl] | (FDR) | (RR:RA:AA) | AAF | Cohort |
| 1 | 1.683 | 0.02202 | Cases: 1060 | 0.00576 | GHS |
| | [1.08, | (0.306) | (1040:20:0) | | Freeze 90 |
| | 2.63] | | Controls: 86140 | | Meta EUR |
| | | | (85159:978:3) | | |

1 = Multiple sclerosis.

Table 6 shows associations of ADCY7 Asp439Glu with altered blood cell counts.

TABLE 6

| Pheno- | Effect | P-value | Cases/Controls | | |
|---|---|---|---|---|---|
| type | [Cl] | (FDR) | (RR:RA:AA) | AAF | Cohort |
| 1 | −0.06627 | 1.563E−06 | Cases: 447144 | 0.00591 | UKB 500K |
| | [−0.0933, | (0.000117) | (441877:5251:16) | | Genotyped |
| | −0.0392] | | | | EUR |
| 2 | −0.05477 | 6.994E−05 | Cases: 447989 | 0.00592 | UKB 500K |
| | [−0.0818, | (0.00346) | (442704:5269:16) | | Genotyped |
| | −0.0278] | | | | EUR |
| 3 | −0.04817 | 0.0004815 | Cases: 446376 | 0.00592 | UKB 500K |
| | [−0.0752, | (0.0204) | (441110:5250:16) | | Genotyped |
| | −0.0211] | | | | EUR |
| 4 | −0.04628 | 0.0007727 | Cases: 448922 | 0.00591 | UKB 500K |
| | [−0.0733, | (0.0259) | (443630:5276:16) | | Genotyped |
| | −0.0193] | | | | EUR |
| 5 | 0.07074 | 3.603E−07 | Cases: 441570 | 0.00589 | UKB 500K |
| | [0.0435, | (3.11E−05) | (436379:5176:15) | | Genotyped |
| | 0.0980] | | | | EUR |

1 = Lymphocyte count.
2 = White blood cell leukocyte count.
3 = Neutrophill count.
4 = Red blood cell erythrocyte count.
5 = Eosinophill count.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

What is claimed is:

1. A method of identifying a subject having an increased risk for developing an interferon mediated disease, wherein the method comprises:
determining or having determined the presence or absence of an Adenylate Cyclase 7 (ADCY7) predicted loss-of-function variant nucleic acid molecule encoding a human ADCY7 polypeptide in a biological sample obtained from the subject;
wherein:
when the subject is ADCY7 reference, then the subject does not have an increased risk for developing an interferon mediated disease; and
when the subject is heterozygous for an ADCY7 predicted loss-of-function variant or homozygous for an ADCY7 predicted loss-of-function variant, then the subject has an increased risk for developing an interferon mediated disease.

2. The method according to claim 1, wherein the ADCY7 predicted loss-of-function variant nucleic acid molecule is a nucleic acid molecule encoding ADCY7 Asp439Glu.

3. The method according to claim 2, wherein the ADCY7 predicted loss-of-function variant nucleic acid molecule is:
a genomic nucleic acid molecule having a nucleotide sequence comprising an adenine at a position corresponding to position 34,648 according to SEQ ID NO:2;
an mRNA molecule having a nucleotide sequence comprising: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10; or
a cDNA molecule produced from an mRNA molecule, wherein the cDNA molecule has a nucleotide sequence comprising: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18.

4. The method according to claim 1, wherein the determining step comprises:
sequencing at least a portion of the nucleotide sequence of the ADCY7 genomic nucleic acid molecule in the biological sample, wherein the sequenced portion comprises a position corresponding to position 34,648 according to SEQ ID NO:2, or the complement thereof; wherein when the sequenced portion of the ADCY7 genomic nucleic acid molecule in the biological sample comprises an adenine at a position corresponding to position 34,648 according to SEQ ID NO:2, then the ADCY7 genomic nucleic acid molecule in the biological sample is an ADCY7 predicted loss-of-function variant genomic nucleic acid molecule;
sequencing at least a portion of the nucleotide sequence of the ADCY7 mRNA molecule in the biological sample, wherein the sequenced portion comprises a position corresponding to: position 1,583 according to SEQ ID NO:7, or the complement thereof; position 1,582 according to SEQ ID NO:8, or the complement thereof; position 1,397 according to SEQ ID NO:9, or the complement thereof; or position 1,344 according to SEQ ID NO:10, or the complement thereof; wherein when the sequenced portion of the ADCY7 mRNA molecule in the biological sample comprises: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10; then the ADCY7 mRNA molecule in the biological sample is an ADCY7 predicted loss-of-function variant mRNA molecule; or
sequencing at least a portion of the nucleotide sequence of the ADCY7 cDNA molecule in the biological sample, wherein the sequenced portion comprises a position corresponding to: position 1,583 according to SEQ ID NO:15, or the complement thereof; position 1,582 according to SEQ ID NO:16, or the complement thereof; position 1,397 according to SEQ ID NO:17, or the complement thereof; or position 1,344 according to SEQ ID NO:18, or the complement thereof; wherein when the sequenced portion of the ADCY7 cDNA molecule in the biological sample comprises: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18; then the ADCY7 cDNA molecule in the biological sample is an ADCY7 predicted loss-of-function variant cDNA molecule.

5. The method according to claim 1, wherein the determining step comprises:
contacting the biological sample with a primer hybridizing to a portion of the nucleotide sequence of the ADCY7 genomic nucleic acid molecule that is proximate to a position corresponding to position 34,648 according to SEQ ID NO:2; extending the primer at least through the position of the nucleotide sequence of the ADCY7 genomic nucleic acid molecule corresponding to position 34,648 according to SEQ ID NO:2; and determining whether the extension product of the primer comprises an adenine at a position corresponding to position 34,648 according to SEQ ID NO:2;
contacting the biological sample with a primer hybridizing to a portion of the nucleotide sequence of the ADCY7 mRNA molecule that is proximate to a position corresponding to: position 1,583 according to SEQ ID NO:7; position 1,582 according to SEQ ID NO:8; position 1,397 according to SEQ ID NO:9; or position 1,344 according to SEQ ID NO:10; extending the primer at least through the position of the nucleotide sequence of the ADCY7 mRNA molecule corresponding to: position 1,583 according to SEQ ID NO:7; position 1,582 according to SEQ ID NO:8; position 1,397 according to SEQ ID NO:9; or position 1,344 according to SEQ ID NO:10; and determining whether the extension product of the primer comprises: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10; or
contacting the biological sample with a primer hybridizing to a portion of the nucleotide sequence of the ADCY7 cDNA molecule that is proximate to a position corresponding to: position 1,583 according to SEQ ID NO:15; position 1,582 according to SEQ ID NO:16; position 1,397 according to SEQ ID NO:17; or position 1,344 according to SEQ ID NO:18; extending the primer at least through the position of the nucleotide sequence of the ADCY7 cDNA molecule corresponding to: position 1,583 according to SEQ ID NO:15; position 1,582 according to SEQ ID NO:16; position 1,397 according to SEQ ID NO:17; or position 1,344 according to SEQ ID NO:18; and determining whether the extension product of the primer comprises: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18.

6. The method according to claim 1, wherein the determining step comprises:
amplifying at least a portion of the nucleic acid molecule that encodes the human ADCY7 polypeptide, wherein the portion comprises an adenine at a position corresponding to position 34,648 according to SEQ ID NO:2, or the complement thereof; labeling the amplified nucleic acid molecule with a detectable label; contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising an adenine at a position corresponding to position 34,648 according to SEQ ID NO:2, or the complement thereof; and detecting the detectable label;
amplifying at least a portion of the nucleic acid molecule that encodes the human ADCY7 polypeptide, wherein the portion comprises: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; labeling the amplified nucleic acid molecule with a detectable label; contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; and detecting the detectable label; or
amplifying at least a portion of the nucleic acid molecule that encodes the human ADCY7 polypeptide, wherein the portion comprises: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; labeling the amplified nucleic acid molecule with a detectable label; contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; and detecting the detectable label.

7. The method according to claim 1, wherein the detecting step comprises:
contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising an adenine at a position corresponding to position 34,648 according to SEQ ID NO:2, or the complement thereof; and detecting the detectable label;
contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; and detecting the detectable label; or
contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; and detecting the detectable label.

8. The method according to claim 1, wherein the subject is heterozygous or homozygous for an ADCY7 predicted loss-of-function variant, and the subject is further administered a therapeutic agent that treats or inhibits the interferon mediated disease.

9. The method according to claim 1, wherein the interferon mediated disease is multiple sclerosis.

10. A method of treating a subject with a therapeutic agent that treats or inhibits an interferon mediated disease, wherein the subject is suffering from an interferon mediated disease, the method comprising the steps of:
determining whether the subject has an Adenylate Cyclase 7 (ADCY7) predicted loss-of-function variant nucleic acid molecule encoding a human ADCY7 polypeptide by:
obtaining or having obtained a biological sample from the subject; and
performing or having performed a genotyping assay on the biological sample to determine if the subject has a genotype comprising the ADCY7 predicted loss-of-function variant nucleic acid molecule; and
when the subject is ADCY7 reference, then administering or continuing to administer to the subject the therapeutic agent that treats or inhibits interferon mediated disease in a standard dosage amount; and
when the subject is heterozygous or homozygous for an ADCY7 predicted loss-of-function variant, then administering or continuing to administer to the subject the therapeutic agent that treats or inhibits interferon mediated disease in an amount that is the same as or greater than a standard dosage amount;
wherein the presence of a genotype having the ADCY7 predicted loss-of-function variant nucleic acid molecule encoding the human ADCY7 polypeptide indicates the subject has an increased risk of developing interferon mediated disease.

11. The method according to claim 10, wherein the ADCY7 predicted loss-of-function variant nucleic acid molecule is a nucleic acid molecule encoding ADCY7 Asp439Glu.

12. The method according to claim 11, wherein the ADCY7 predicted loss-of-function variant nucleic acid molecule is:
a genomic nucleic acid molecule having a nucleotide sequence comprising an adenine at a position corresponding to position 34,648 according to SEQ ID NO:2;
an mRNA molecule having a nucleotide sequence comprising: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10; or
a cDNA molecule produced from an mRNA molecule, wherein the cDNA molecule has a nucleotide sequence comprising: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18.

13. The method according to claim 10, wherein the genotyping assay comprises:
sequencing at least a portion of the nucleotide sequence of the ADCY7 genomic nucleic acid molecule in the biological sample, wherein the sequenced portion comprises a position corresponding to position 34,648 according to SEQ ID NO:2, or the complement thereof; wherein when the sequenced portion of the ADCY7 genomic nucleic acid molecule in the biological sample comprises an adenine at a position corresponding to position 34,648 according to SEQ ID NO:2, then the ADCY7 genomic nucleic acid molecule in the biological sample is an ADCY7 predicted loss-of-function variant genomic nucleic acid molecule;
sequencing at least a portion of the nucleotide sequence of the ADCY7 mRNA molecule in the biological sample, wherein the sequenced portion comprises a position corresponding to: position 1,583 according to SEQ ID NO:7, or the complement thereof; position 1,582 according to SEQ ID NO:8, or the complement thereof; position 1,397 according to SEQ ID NO:9, or the complement thereof; or position 1,344 according to SEQ ID NO:10, or the complement thereof; wherein when the sequenced portion of the ADCY7 mRNA molecule in the biological sample comprises: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10; then the ADCY7 mRNA molecule in the biological sample is an ADCY7 predicted loss-of-function variant mRNA molecule; or
sequencing at least a portion of the nucleotide sequence of the ADCY7 cDNA molecule in the biological sample, wherein the sequenced portion comprises a position corresponding to: position 1,583 according to SEQ ID NO:15, or the complement thereof; position 1,582 according to SEQ ID NO:16, or the complement thereof; position 1,397 according to SEQ ID NO:17, or the complement thereof; or position 1,344 according to SEQ ID NO:18, or the complement thereof; wherein when the sequenced portion of the ADCY7 cDNA molecule in the biological sample comprises: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18; then the ADCY7 cDNA molecule in the biological sample is an ADCY7 predicted loss-of-function variant cDNA molecule.

14. The method according to claim 10, wherein the genotyping assay comprises:
contacting the biological sample with a primer hybridizing to a portion of the nucleotide sequence of the ADCY7 genomic nucleic acid molecule that is proximate to a position corresponding to position 34,648 according to SEQ ID NO:2; extending the primer at least through the position of the nucleotide sequence of the ADCY7 genomic nucleic acid molecule corresponding to position 34,648 according to SEQ ID NO:2; and determining whether the extension product of the primer comprises an adenine at a position corresponding to position 34,648 according to SEQ ID NO:2;
contacting the biological sample with a primer hybridizing to a portion of the nucleotide sequence of the ADCY7 mRNA molecule that is proximate to a position corresponding to: position 1,583 according to SEQ ID NO:7; position 1,582 according to SEQ ID NO:8; position 1,397 according to SEQ ID NO:9; or position 1,344 according to SEQ ID NO:10; extending the primer at least through the position of the nucleotide sequence of the ADCY7 mRNA molecule corresponding to: position 1,583 according to SEQ ID NO:7; position 1,582 according to SEQ ID NO:8; position 1,397 according to SEQ ID NO:9; or position 1,344 according to SEQ ID NO:10; and determining whether the extension product of the primer comprises: an adenine at a position corresponding to position 1,583 according to SEQ ID NO:7; an adenine at a position corresponding to position 1,582 according to SEQ ID NO:8; an adenine at a position corresponding to position 1,397 according to SEQ ID NO:9; or an adenine at a position corresponding to position 1,344 according to SEQ ID NO:10; or
contacting the biological sample with a primer hybridizing to a portion of the nucleotide sequence of the ADCY7 cDNA molecule that is proximate to a position corresponding to: position 1,583 according to SEQ ID NO:15; position 1,582 according to SEQ ID NO:16; position 1,397 according to SEQ ID NO:17; or position 1,344 according to SEQ ID NO:18; extending the primer at least through the position of the nucleotide sequence of the ADCY7 cDNA molecule corresponding to: position 1,583 according to SEQ ID NO:15; position 1,582 according to SEQ ID NO:16; position 1,397 according to SEQ ID NO:17; or position 1,344 according to SEQ ID NO:18; and determining whether the extension product of the primer comprises: an adenine at a position corresponding to position 1,583 according to SEQ ID NO:15; an adenine at a position corresponding to position 1,582 according to SEQ ID NO:16; an adenine at a position corresponding to position 1,397 according to SEQ ID NO:17; or an adenine at a position corresponding to position 1,344 according to SEQ ID NO:18.

15. The method according to claim 10, wherein the genotyping assay comprises:
amplifying at least a portion of the nucleic acid molecule that encodes the human ADCY7 polypeptide, wherein the portion comprises an adenine at a position corresponding to position 34,648 according to SEQ ID NO:2, or the complement thereof; labeling the amplified nucleic acid molecule with a detectable label; contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising an adenine at a position corresponding to position 34,648 according to SEQ ID NO:2, or the complement thereof; and detecting the detectable label;
amplifying at least a portion of the nucleic acid molecule that encodes the human ADCY7 polypeptide, wherein the portion comprises: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; labeling the amplified nucleic acid molecule with a detectable label; contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; and detecting the detectable label; or
amplifying at least a portion of the nucleic acid molecule that encodes the human ADCY7 polypeptide, wherein the portion comprises: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; labeling the amplified nucleic acid molecule with a detectable label; contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; and detecting the detectable label.

16. The method according to claim 10, wherein the genotyping assay comprises:
contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising an adenine at a position corresponding to position 34,648 according to SEQ ID NO:2, or the complement thereof; and detecting the detectable label;
contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; and detecting the detectable label; or
contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof; and detecting the detectable label.

17. The method according to claim 10, wherein the nucleic acid molecule is present within a cell obtained from the subject.

18. The method according to claim 10, wherein the interferon mediated disease is multiple sclerosis.

19. A method of detecting a human Adenylate Cyclase 7 (ADCY7) variant nucleic acid molecule in a subject comprising assaying a sample obtained from the subject to determine whether a nucleic acid molecule in the sample is:
a genomic nucleic acid molecule comprising a nucleotide sequence comprising: an adenine at a position corresponding to position 34,648 according to SEQ ID NO:2, or the complement thereof;
an mRNA molecule comprising a nucleotide sequence comprising: an adenine at a position corresponding to position 1,583 according to SEQ ID NO:7, or the complement thereof; an adenine at a position corresponding to position 1,582 according to SEQ ID NO:8, or the complement thereof; an adenine at a position corresponding to position 1,397 according to SEQ ID NO:9, or the complement thereof; or an adenine at a position corresponding to position 1,344 according to SEQ ID NO:10, or the complement thereof; or
a cDNA molecule comprising a nucleotide sequence comprising: an adenine at a position corresponding to position 1,583 according to SEQ ID NO:15, or the complement thereof; an adenine at a position corresponding to position 1,582 according to SEQ ID NO:16, or the complement thereof; an adenine at a position corresponding to position 1,397 according to SEQ ID NO:17, or the complement thereof; or an adenine at a position corresponding to position 1,344 according to SEQ ID NO:18, or the complement thereof.

20. A method of detecting the presence of a human Adenylate Cyclase 7 (ADCY7) Asp439Glu variant polypeptide, comprising performing an assay on a sample obtained from a subject to determine whether an ADCY7 protein in the sample comprises: a glutamic acid at a position corresponding to position 439 according to SEQ ID NO:21; or a glutamic acid at a position corresponding to position 439 according to SEQ ID NO:22.
